(19) United States
(12) Patent Application Publication
(10) Pub. No.: US 2023/0405046 A1
(43) Pub. Date: Dec. 21, 2023

(54) ANTIGEN-SPECIFIC T CELL RECEPTORS AND T CELL EPITOPES

(71) Applicant: BioNTech Cell & Gene Therapies GmbH, Mainz (DE)

(72) Inventors: Ugur SAHIN, Mainz (DE); Petra OEHM, Mainz (DE); Tana OMOKOKO, Mainz (DE); Andrea BREITKREUZ, Mainz (DE); Claudia TOLLIVER, Mainz (DE); Janina Patricia CASPAR, Mainz (DE); Lisa HEBICH, Mainz (DE); Anke OELBERMANN, Mainz (DE); Bettina STEEGE, Mainz (DE); Evelyna DERHOVANESSIAN, Mainz (DE); Sebastian NEWRZELA, Mainz (DE); Rene ROTH, Mainz (DE)

(21) Appl. No.: 17/906,448

(22) PCT Filed: Mar. 15, 2021
(86) PCT No.: PCT/EP2021/056559

§ 371 Date: Sep. 15, 2022

(30) Foreign Application Priority Data
Mar. 16, 2020 (WO) PCT/EP2020/057108

(51) Int. Cl. A61K 35/17; A61K 39/00; C07K 14/725; A61P 37/02; C12N 15/11

(52) U.S. Cl. CPC A61K 35/17; A61K 39/4611; C07K 14/7051; A61P 37/02; A61K 39/464411; C12N 15/11

(57) ABSTRACT

The present invention relates to the provision of clinically relevant T cell receptors and T cell epitopes which are useful for immunotherapy. The present invention also relates to therapies involving immune effector cells such as T cells expressing a T cell receptor disclosed herein and/or being specific for a T cell epitope disclosed herein.

### CROSS-REFERENCE TO RELATED APPLICATIONS

This application is a National Stage Entry of International Application Number PCT/EP2021/056559, which was filed on Mar. 15, 2021 and claimed priority to International Application Number PCT/EP2020/057108, which was filed on Mar. 16, 2020. The contents of each of the aforementioned applications are incorporated herein by reference in their entireties.

### SEQUENCE LISTING

This instant application contains a Sequence Listing which has been submitted in ASCII format via EFS-Web and is hereby incorporated by reference in its entirety. Said ASCII copy, created on Sep. 2, 2022 and updated on Feb. 9, 2023, is named 028320-8044 Sequence Listing.txt, and is 86,016 bytes in size.

### TECHNICAL FIELD OF THE INVENTION

The present invention relates to the provision of clinically relevant T cell receptors and T cell epitopes which are useful for immunotherapy. The present invention also relates to therapies involving immune effector cells such as T cells expressing a T cell receptor disclosed herein and/or being specific for a T cell epitope disclosed herein. In one embodiment, the immune effector cells are engineered to express the T cell receptor, e.g., genetically modified to express the T cell receptor. Such genetic modification may be effected ex vivo or in vitro and subsequently the immune effector cells may be administered to a subject in need of treatment, or may be effected in vivo in a subject in need of treatment. These methods are, in particular, useful for the treatment of cancers characterized by diseased cells expressing an antigen the T cell receptor is directed to. The T cell receptor-engineered immune effector cells may be provided to a subject by administering the T cell receptor-engineered immune effector cells or by generating the T cell receptor-engineered immune effector cells in the subject. Furthermore, target antigen for the T cell receptor may be provided to a subject by administering to the subject an antigen targeted by the T cell receptor, a polynucleotide encoding the antigen, or cells expressing the antigen. The antigen to which the T cell receptor is targeted may comprise a naturally occurring antigen or a variant thereof, or a fragment of the naturally occurring antigen or variant thereof. In one particularly preferred embodiment, the polynucleotide encoding the antigen is RNA. The methods and agents described herein are, in particular, useful for the treatment of diseases characterized by diseased cells expressing an antigen the T cell receptor or T cell receptor-engineered immune effector cells are directed to.

### BACKGROUND OF THE INVENTION

The immune system plays an important role in cancer, autoimmunity, allergy as well as in pathogen-associated diseases. T cells and NK cells are important mediators of anti-tumor immune responses. CD8⁺ T cells and NK cells can directly lyse tumor cells. CD4⁺ T cells, on the other hand, can mediate the influx of different immune subsets including CD8⁺ T cells and NK cells into the tumor. CD4⁺ T cells are able to license dendritic cells (DCs) for the priming of anti-tumor CD8⁺ T cell responses and can act directly on tumor cells via IFNγ mediated MHC upregulation and growth inhibition. CD8⁺ as well as CD4⁺ tumor specific T cell responses can be induced via vaccination or by adoptive transfer of T cells.

The recognition and binding of a particular antigen by T cells is mediated by T cell receptors (TCRs) expressed on the surface of T cells. The T cell receptor of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell.

MHC and antigen binding is mediated by the complementary determining regions 1, 2 and 3 (CDR1, CDR2, CDR3) of the TCR. The CDR3 of the β-chain is most critical for antigen recognition.

Active immunization tends to induce and expand antigen-specific T cells in the patient, which are able to specifically recognize and kill diseased cells. In contrast, passive immunization may rely on the adoptive transfer of T cells, which were expanded and optional genetically engineered in vitro (adoptive T cell therapy).

Different antigen formats can be used for active immunization (vaccination) including whole cancer cells, proteins, peptides or immunizing vectors such as RNA, DNA or viral vectors that can be applied either directly in vivo or in vitro by pulsing of DCs following transfer into the patient.

Adoptive cell transfer (ACT) based immunotherapy can be broadly defined as a form of passive immunization with previously sensitized T cells that are transferred to non-immune recipients or to the autologous host after ex vivo expansion from low precursor frequencies to clinically relevant cell numbers.

Cell types that have been used for ACT experiments are lymphokine-activated killer (LAK) cells (Mule, J. J. et al. (1984) Science 225, 1487-1489; Rosenberg, S. A. et al. (1985) N. Engl. J. Med. 313, 1485-1492), tumor-infiltrating lymphocytes (TILs) (Rosenberg, S. A. et al. (1994) J. Natl. Cancer Inst. 86, 1159-1166), donor lymphocytes after hematopoietic stem cell transplantation (HSCT) as well as tumor-specific T cell lines or clones (Dudley, M. E. et al. (2001) J. Immunother. 24, 363-373; Yee, C. et al. (2002) Proc. Natl. Acad. Sci. U.S.A 99, 16168-16173). An alternative approach is the adoptive transfer of autologous T cells reprogrammed to express a tumor-reactive immunoreceptor of defined specificity during short-time ex vivo culture followed by reinfusion into the patient (Kershaw M. H. et al. (2013) Nature Reviews Cancer 13 (8):525-41). This strategy makes ACT applicable to a variety of common malignancies even if tumor-reactive T cells are absent in the patient.

Shared, non-mutated tumour-associated antigens (TAA) such as cancer/germline genes or lineage-specific differentiation markers are frequently expressed across human cancer types and represent attractive immunotherapy targets (Coulie, P. G., et al., Nat. Rev. Cancer 14, 135-146 (2014)). TAA are considered to be subject to central T cell tolerance (Kyewski, B. & Derbinski, J., Nat. Rev. Immunol. 4, 688-98 (2004)), which may contribute to the largely weak, clinically ineffective T cell responses observed hitherto in the vast majority of vaccine trials (Melero, I. et al., Nat. Rev. Clin. Oncol. 11, 509-524 (2014); Romero, P. et al., Sci. Transl. Med. 8, 334ps9 (2016)). We have recently introduced a systemically administered, nano-particulate liposomal RNA vaccine class (RNA-LPX) for body-wide targeting of dendritic cells (DC) in lymphoid compartments. Temporospatially aligned to delivering the vaccine antigen, RNA-LPX mediates potent co-stimulation through type-I interferon driven antiviral immune mechanisms, which results in profound expansion of anti-tumour effector T cells even against self-antigens (Kranz, L. M. et al., Nature 534, 396-401 (2016); De Vries, J. & Figdor, C., Nature 534, 329-31 (2016)). For first-in-human testing of this approach we initiated a phase I dose-escalation trial in advanced melanoma patients who had radiologically evaluable metastatic disease or resected non-evaluable disease at baseline (Lipo-MERIT, NCT02410733).

The vaccine (referred to as melanoma FixVac) is composed of four lipid-complexed RNAs encoding the non-mutant TAAs NY-ESO-1, MAGE-A3, tyrosinase and TPTE, each of which is known for its restricted expression in normal tissues, high immunogenicity and high prevalence in human melanoma (Simon, P. et al., Cancer Immunol. Res. 2, 1230-44 (2014); Cheever, M. A. et al., Clin. Cancer Res. 15, 5323-37 (2009)). The single-stranded, 5′-capped vaccine messenger RNA (FIG. 1A) bears functional sequence elements that improve efficiency of its translation specifically in immature DCs (Holtkamp, S. et al., Blood 108, 4009-17 (2006); Orlandini von Niessen, A. G. et al., Mol. Ther. 27, 824-836 (2018)). The open reading frame of each TAA is fused in frame to an N-terminal secretion signal and C-terminally to tetanus toxoid helper epitopes and an endosomal targeting domain, which augments processing and presentation of tumour antigen-derived epitopes on the patient's individual HLA-class I and II molecules (Kreiter, S. et al., J. Immunol. 180, 309-318 (2008)).

Patients who express at least one of the TAAs confirmed per qRT-PCR analysis were eligible for this trial. Melanoma FixVac was administered according to a prime/repeat boosts protocol followed by optional continued monthly treatment (FIG. 1B). In the dose escalation part target dose levels in the range of 7.2 μg and 400 μg total RNA were tested. Patients within dose escalation cohorts were stepped up to their target dose by intra-individual dose-escalation in weekly intervals. The expansion part included three dose cohorts with melanoma FixVac alone, and patients who were treated with melanoma FixVac in combination with anti-PD1 or BRAF/MEK inhibitors.

Melanoma FixVac, as a single agent and in combination with anti-PD1 therapy, mediated durable objective responses in heavily pre-treated with metastatic and progressing tumours. Clinical responses correlated with the induction of HLA class I and II restricted TAA-specific T cell responses. The characterization of the corresponding HLA class I restricted TCRs confirmed that they mediate recognition and lysis of endogenously TAA-expressing tumor cell lines after transfer in CD8⁺ T cells from healthy donors. Similarly, HLA class II restricted TCRs were functional after transfer in CD4⁺ T cells of healthy donors.

The majority of vaccine-induced or vaccine-amplified CD4⁺ or CD8⁺ T-cell responses were detected against NY-ESO-1 and MAGE-A3, both known for its restricted expression in normal tissues, high immunogenicity and high prevalence in human melanoma (Simon, P. et al., Cancer Immunol. Res. 2, 1230-44 (2014); Cheever, M. A. et al., Clin. Cancer Res. 15, 5323-37 (2009)). The corresponding TCRs are expected to be of therapeutic value in the setting of TCR gene therapy. Furthermore, the novel HLA-B*4001-restricted epitope NY-ESO-1₁₂₄₋₁₃₃ was discovered and confirmed to be processed and presented by endogenously NY-ESO-1 expressing melanoma cells.

### DESCRIPTION OF INVENTION

### Summary of the Invention

Immunotherapeutic strategies are promising options for the treatment of cancer. The exact definition of peptide epitopes derived from tumor antigens may contribute to improve specificity and efficiency of vaccination strategies. Furthermore, by adoptive transfer of T cells engineered to express a defined antigen-specific T cell receptor (TCR) tumor-associated antigens can be specifically targeted thereby leading to selective destruction of malignant cells.

The present invention relates to T cell receptors specific for the tumor-associated antigen NY-ESO-1, MAGE-A3, tyrosinase, and KRAS, respectively, in particular when presented on the surface of a cell such as a diseased cell or an antigen-presenting cell, as well as peptides comprising epitopes recognized by these T cell receptors.

In one aspect, the invention relates to a peptide comprising an amino acid sequence selected from the group consisting of SEQ ID NOs: 39 to 44, 101, and 102 or a variant of said amino acid sequence.

In one embodiment, the peptide has a length of 200 amino acids or less, 150 amino acids or less, 100 amino acids or less, 50 amino acids or less, 40 amino acids or less, 30 amino acids or less, 20 amino acids or less, or 15 amino acids or less.

In one embodiment, the peptide is a MHC class I or class II presented peptide, preferably a MHC class I presented peptide, or, if present within cells, can be processed to produce a procession product thereof, which is a MHC class I or class II presented peptide, preferably a MHC class I presented peptide. Preferably, said MHC class I or class II presented peptide has a sequence substantially corresponding to the given amino acid sequence, i.e., an amino acid sequence selected from the group consisting of SEQ ID NOs: 39 to 44, 101, and 102 or a variant of said amino acid sequence. Preferably, a peptide according to the invention is capable of stimulating a cellular response against a disease involving cells characterized by presentation of an antigen from which the peptide is derived, i.e., NY-ESO-1, MAGE-A3, tyrosinase, and KRAS, respectively, with class I MHC.

In a further aspect, the invention relates to a nucleic acid encoding the peptide of the invention.

Such nucleic acid may be present in a plasmid or an expression vector and may be functionally linked to a promoter. In one embodiment, the nucleic acid is RNA.

In a further aspect, the invention relates to a cell, which is genetically modified to express the peptide of the invention.

The cell may be a recombinant cell and may secrete the encoded peptide or a procession product thereof, may express it on the surface and preferably may additionally express an MHC molecule which binds to said peptide or a procession product thereof and preferably presents said peptide or a procession product thereof on the cell surface. In one embodiment, the cell expresses the MHC molecule endogenously. In a further embodiment, the cell expresses the MHC molecule and/or the peptide in a recombinant manner. The cell is preferably nonproliferative. In a preferred embodiment, the cell is an antigen-presenting cell, in particular a dendritic cell, a monocyte or a macrophage.

In one embodiment, the cell comprises nucleic acid encoding the peptide.

In one embodiment, the cell presents the peptide or a procession product thereof. The procession product may be a peptide having the given amino acid sequence, i.e. an amino acid sequence selected from the group consisting of SEQ ID NOs: 39 to 44, 101, and 102 or a variant of said amino acid sequence.

In a further aspect, the invention relates to a cell that presents the peptide of the invention or a procession product thereof.

The procession product may be a peptide having the given amino acid sequence, i.e. an amino acid sequence selected from the group consisting of SEQ ID NOs: 39 to 44, 101, and 102 or a variant of said amino acid sequence. The cell may present the peptide or a procession product thereof by MHC molecules on its surface. In one embodiment, the cell endogenously expresses an MHC molecule. In a further embodiment, the cell recombinantly expresses an MHC molecule. In one embodiment, the MHC molecules of the cell are loaded (pulsed) with the peptide by addition of the peptide to the cell. The cell may recombinantly express the peptide and present said peptide or a procession product thereof on the cell surface. The cell is preferably nonproliferative. In a preferred embodiment, the cell is an antigen-presenting cell such as a dendritic cell, a monocyte or a macrophage.

In a further aspect, the invention relates to an immune effector cell, which is reactive with a peptide of the invention.

In one embodiment, the immune effector cell is reactive with a peptide of the invention when presented on the surface of a cell. The immune effector cell may be a cell that has been sensitized in vitro to recognize the peptide. The immune effector cell may be a T cell, preferably a cytotoxic T cell. Preferably, the immune effector cell binds to a sequence in the peptide substantially corresponding to the given amino acid sequence, i.e., an amino acid sequence selected from the group consisting of SEQ ID NOs: 39 to 44, 101, and 102 or a variant of said amino acid sequence.

In a further aspect, the invention relates to a T cell receptor, which is reactive with a peptide of the invention, or a polypeptide chain of said T cell receptor.

In a further aspect, the invention relates to a T cell receptor polypeptide or a T cell receptor comprising said T cell receptor polypeptide,

wherein said T cell receptor polypeptide is selected from the group consisting of:
(i) a T cell receptor polypeptide comprising at least one, preferably two, more preferably all three of the CDR sequences of a T cell receptor α-chain selected from SEQ ID NOs: 5, 7, 9, 11, 13, 15, 17, 19, 21, 23, 25, 27, 29, 31, 33, 35, 37, 91, 93, 95, 97, and 99 or a variant thereof and
(ii) a T cell receptor polypeptide comprising a T cell receptor α-chain sequence selected from SEQ ID NOs: 5, 7, 9, 11, 13, 15, 17, 19, 21, 23, 25, 27, 29, 31, 33, 35, 37, 91, 93, 95, 97, and 99 or a variant thereof.

In one embodiment, the T cell receptor polypeptide is a T cell receptor α-chain.

In one embodiment, the invention relates to a T cell receptor α-chain or a T cell receptor comprising said T cell receptor α-chain,

wherein said T cell receptor α-chain is selected from the group consisting of:
(i) a T cell receptor α-chain comprising at least one, preferably two, more preferably all three of the CDR sequences of a T cell receptor α-chain selected from SEQ ID NOs: 5, 7, 9, 11, 13, 15, 17, 19, 21, 23, 25, 27, 29, 31, 33, 35, 37, 91, 93, 95, 97, and 99 or a variant thereof and
(ii) a T cell receptor α-chain comprising a T cell receptor α-chain sequence selected from SEQ ID NOs: 5, 7, 9, 11, 13, 15, 17, 19, 21, 23, 25, 27, 29, 31, 33, 35, 37, 91, 93, 95, 97, and 99 or a variant thereof.

The CDR sequences are shown underlined in the sequences of the above mentioned T cell receptor α-chains given herein.

In a further aspect, the invention relates to a T cell receptor polypeptide or a T cell receptor comprising said T cell receptor polypeptide,

wherein said T cell receptor polypeptide is selected from the group consisting of:
(i) a T cell receptor polypeptide comprising at least one, preferably two, more preferably all three of the CDR sequences of a T cell receptor β-chain selected from SEQ ID NOs: 6, 8, 10, 12, 14, 16, 18, 20, 22, 24, 26, 28, 30, 32, 34, 36, 38, 92, 94, 96, 98, and 100 or a variant thereof
and
(ii) a T cell receptor polypeptide comprising a T cell receptor β-chain sequence selected from SEQ ID NOs: 6, 8, 10, 12, 14, 16, 18, 20, 22, 24, 26, 28, 30, 32, 34, 36, 38, 92, 94, 96, 98, and 100 or a variant thereof.

In one embodiment, the T cell receptor polypeptide is a T cell receptor β-chain.

In one embodiment, the invention relates to a T cell receptor β-chain or a T cell receptor comprising said T cell receptor β-chain, wherein said T cell receptor β-chain is selected from the group consisting of:
(i) a T cell receptor β-chain comprising at least one, preferably two, more preferably all three of the CDR sequences of a T cell receptor β-chain selected from SEQ ID NOs: 6, 8, 10, 12, 14, 16, 18, 20, 22, 24, 26, 28, 30, 32, 34, 36, 38, 92, 94, 96, 98, and 100 or a variant thereof
and
(ii) a T cell receptor β-chain comprising a T cell receptor β-chain sequence selected from SEQ ID NOs: 6, 8, 10, 12, 14, 16, 18, 20, 22, 24, 26, 28, 30, 32, 34, 36, 38, 92, 94, 96, 98, and 100 or a variant thereof.

The CDR sequences are shown underlined in the sequences of the above mentioned T cell receptor β-chains given herein.

In a further aspect, the invention relates to a T cell receptor selected from the group consisting of:
(I) a T cell receptor comprising:
(i) at least one, preferably two, more preferably all three of the CDR sequences of the T cell receptor α-chain of SEQ ID NO: x or a variant thereof, and
(ii) at least one, preferably two, more preferably all three of the CDR sequences of a T cell receptor β-chain of SEQ ID NO: x+1 or a variant thereof;
   wherein x selected from 5, 7, 9, 11, 13, 15, 17, 19, 21, 23, 25, 27, 29, 31, 33, 35, 37, 91, 93, 95, 97, and 99
   and
(II) a T cell receptor comprising:
(i) the T cell receptor α-chain sequence of SEQ ID NO: x or a variant thereof, and
(ii) the T cell receptor β-chain sequence of SEQ ID NO: x+1 or a variant thereof;
   wherein x selected from 5, 7, 9, 11, 13, 15, 17, 19, 21, 23, 25, 27, 29, 31, 33, 35, 37, 91, 93, 95, 97, and 99.

In one embodiment, the invention relates to a T cell receptor selected from the group consisting of:
(I) a T cell receptor comprising:
(i) a T cell receptor α-chain comprising at least one, preferably two, more preferably all three of the CDR sequences of the T cell receptor α-chain of SEQ ID NO: x or a variant thereof, and
(ii) a T cell receptor β-chain comprising at least one, preferably two, more preferably all three of the CDR sequences of a T cell receptor β-chain of SEQ ID NO: x+1 or a variant thereof;
wherein x selected from 5, 7, 9, 11, 13, 15, 17, 19, 21, 23, 25, 27, 29, 31, 33, 35, 37, 91, 93, 95, 97, and 99
   and
(II) a T cell receptor comprising:
(i) a T cell receptor α-chain comprising the T cell receptor α-chain sequence of SEQ ID NO: x or a variant thereof, and
(ii) a T cell receptor β-chain comprising the T cell receptor β-chain sequence of SEQ ID NO: x+1 or a variant thereof;
   wherein x selected from 5, 7, 9, 11, 13, 15, 17, 19, 21, 23, 25, 27, 29, 31, 33, 35, 37, 91, 93, 95, 97, and 99.

The above T cell receptors are preferably specific for the tumor-associated antigen NY-ESO-1, MAGE-A3, tyrosinase and KRAS, respectively, in particular when presented on the surface of a cell such as a diseased cell or an antigen-presenting cell.

In a further aspect, the invention relates to a nucleic acid encoding the T cell receptor chain or T cell receptor of the invention.

In a further aspect, the invention relates to a cell which is genetically modified to express the T cell receptor chain or T cell receptor of the invention.

In one embodiment, the cell comprises nucleic acid encoding the T cell receptor chain or T cell receptor.

The cell may be an effector or stem cell, preferably an immune effector cell. In one embodiment, the cell is an immune effector cell. The immune effector cell may be a T cell, preferably a cytotoxic T cell. Preferably, the immune effector cell is reactive with the tumor-associated antigen NY-ESO-1, MAGE-A3, tyrosinase and KRAS, respectively, in particular when presented on the surface of a cell such as a diseased cell or an antigen-presenting cell, and specifically with a peptide of the invention and preferably binds to a sequence in the peptide substantially corresponding to the given amino acid sequence, i.e., an amino acid sequence selected from the group consisting of SEQ ID NOs: 39 to 44, 101, and 102 or a variant of said amino acid sequence.

In a further aspect, the invention relates to a method for preparing immune effector cells genetically modified to express the T cell receptor of the invention, comprising delivering nucleic acid encoding the T cell receptor to the immune effector cells.

In one embodiment, the method comprises contacting the immune effector cells with particles comprising the nucleic acid. In one embodiment, said particles further comprise a targeting molecule for targeting the immune effector cells. In one embodiment, contacting the immune effector cells with the particles delivers the nucleic acid to the immune effector cells.

In one embodiment, the immune effector cells to be genetically modified are present in vivo or in vitro.

In one embodiment, the immune effector cells to be genetically modified are present in vivo in a subject and the method comprises administering the particles to the subject.

Furthermore, the present invention generally embraces the treatment of diseases by targeting diseased cells expressing the antigen NY-ESO-1, MAGE-A3, tyrosinase and KRAS, respectively. The treatment described herein may be a therapeutic or prophylactic treatment of a malignant disease.

The methods may provide for the selective eradication of cells that present the tumor antigen NY-ESO-1, MAGE-A3, tyrosinase and KRAS, respectively, thereby minimizing adverse effects to normal cells not presenting said antigen. Thus, preferred diseases for a therapy are those in which at least one of the antigens described herein are expressed and presented such as malignant diseases, in particular cancer diseases such as those described herein. The target cells may express the antigen in the context of MHC for recognition by a T cell receptor (TCR).

In one embodiment, immune effector cells genetically modified to express a T cell receptor (TCR) targeting the cells through binding to the antigen (or a procession product thereof) may be provided to a subject such as by administration of genetically modified immune effector cells to the subject or generation of genetically modified immune effector cells in the subject. Genetic modification may be achieved using particles comprising nucleic acid encoding a T cell receptor for genetic modification and optionally a targeting molecule for targeting the immune effector cells. The particles may deliver the nucleic acid to cells in vitro/ex vivo as well as in vivo. A vaccine antigen (which may be the disease-associated antigen or a variant thereof (e.g. a peptide or protein comprising an epitope of the disease-associated antigen, in particular an epitope recognized by the TCR, such as the given amino acid sequence, i.e. an amino acid sequence selected from the group consisting of SEQ ID NOs: 39 to 44, 101, and 102 or a variant of said amino acid sequence), nucleic acid coding therefor, or cells expressing the antigen may be administered to provide (optionally following expression of the nucleic acid by appropriate target cells) antigen for stimulation, priming and/or expansion of immune effector cells genetically modified to express an antigen receptor, wherein the immune effector cells are targeted to the antigen or a procession product thereof. In one embodiment, the polynucleotide encoding the vaccine antigen is RNA. In one embodiment, vaccine antigen-encoding RNA is targeted to secondary lymphoid organs. Immune effector cells such as T cells stimulated, primed and/or expanded in the patient are able to recognize cells expressing an antigen resulting in the eradication of diseased cells. In one embodiment, the immune effector cells are CD8⁺ T cells. In one embodiment, the targeting molecules described herein bind to the CD8 receptor on CD8⁺ T cells. In one embodiment, the immune effector cells are CD4⁺ T cells. In one embodiment, the targeting molecules described herein bind to the CD4 receptor on CD4⁺ T cells. In one embodiment, the immune effector cells are CD4⁺ T cells and/or CD8⁺ T cells. In one embodiment, the targeting molecules described herein bind to the CD3 on T cells. In one embodiment, the immune effector cells are directed against a tumor or cancer. In one embodiment, the target cell population or target tissue is tumor cells or tumor tissue, in particular of a solid tumor. In one embodiment, the target antigen is a tumor antigen.

The methods and agents described herein are, in particular, useful for the treatment of diseases characterized by diseased cells expressing an antigen the immune effector cells are directed to, i.e. the tumor antigen NY-ESO-1, MAGE-A3, tyrosinase and KRAS, respectively. Preferably, a cell is genetically modified to stably express a T cell receptor on its surface. In one embodiment, immune effector cells either from a subject to be treated or from a different subject are administered to the subject to be treated. The administered immune effector cells may be genetically modified ex vivo prior to administration or genetically modified in vivo in the subject following administration to express a T cell receptor described herein. In one embodiment, the immune effector cells are endogenous in a subject to be treated (thus, are not administered to the subject to be treated) and are genetically modified in vivo in the subject to express a T cell receptor described herein. Accordingly, immune effector cells may be genetically modified, ex vivo or in vivo, to express a T cell receptor. Thus, such genetic modification with T cell receptor may be effected in vitro and subsequently the immune effector cells administered to a subject in need of treatment or may be effected in vivo in a subject in need of treatment.

In a further aspect, the invention relates to a pharmaceutical composition comprising one or more of:
(i) the peptide of the invention;
(ii) the nucleic acid of the invention;
(iii) the cell of the invention; and
(iv) the immune effector cell of the invention.

A pharmaceutical composition of the invention may comprise a pharmaceutically acceptable carrier and may optionally comprise one or more adjuvants, stabilizers etc. The pharmaceutical composition may in the form of a therapeutic or prophylactic vaccine. In one embodiment, the pharmaceutical composition is for use in treating or preventing a malignant disease such as those described herein.

Administration of a pharmaceutical composition as described above may provide MHC class II-presented epitopes that are capable of eliciting a CD4+ helper T cell response and/or a CD8+ T cell response against antigens described herein. Alternatively or additionally, administration of a pharmaceutical composition as described above may provide MHC class I-presented epitopes that are capable of eliciting a CD8+ T cell response against antigens described herein.

In one embodiment, the antigen concerned is NY-ESO-1, MAGE-A3, tyrosinase and KRAS, respectively, and the pharmaceutical composition of the present invention is useful in the treatment and/or prevention of a malignant disease.

In a further aspect, the invention relates to a method for treating a subject, comprising administering the pharmaceutical composition of the invention to the subject.

In a further aspect, the invention relates to a method for treating a subject comprising providing immune effector cells genetically modified to express the T cell receptor of the invention to the subject.

In one embodiment, the method of the above aspects is a method of inducing an immune response in said subject. In one embodiment, the immune response is a T cell-mediated immune response. In one embodiment, the immune response is an immune response to a target cell population or target tissue expressing an antigen. In one embodiment, the target cell population or target tissue is cancer cells or cancer tissue. In one embodiment, the cancer cells or cancer tissue is solid cancer.

In a further aspect, the invention relates to a method for treating a subject having a disease, disorder or condition associated with expression or elevated expression of an antigen comprising providing immune effector cells genetically modified to express the T cell receptor of the invention, the T cell receptor being targeted to the antigen associated with the disease, disorder or condition or cells expressing the antigen associated with the disease, disorder or condition, to the subject.

In one embodiment, the disease, disorder or condition is cancer and the antigen associated with the disease, disorder or condition is a tumor antigen. In one embodiment, the antigen concerned is NY-ESO-1, MAGE-A3, tyrosinase and KRAS, respectively, and the method of the present invention is useful in the treatment and/or prevention of a malignant disease.

In one embodiment, the disease, disorder or condition is solid cancer.

In one embodiment of the method of the above aspects, the immune effector cells genetically modified to express the T cell receptor are provided to the subject by administering the immune effector cells genetically modified to express the T cell receptor or by generating the immune effector cells genetically modified to express the T cell receptor in the subject.

In one embodiment of the method of the above aspects, the immune effector cells genetically modified to express the T cell receptor are prepared by a method comprising delivering nucleic acid encoding the T cell receptor to the immune effector cells.

In one embodiment of the method of the above aspects, the immune effector cells genetically modified to express the T cell receptor are prepared by a method comprising contacting the immune effector cells with particles comprising nucleic acid encoding the T cell receptor. In one embodiment, said particles further comprise a targeting molecule for targeting the immune effector cells. In one embodiment, contacting the immune effector cells with the particles delivers the nucleic acid to the immune effector cells.

In one embodiment of the method of the above aspects, the immune effector cells to be genetically modified are present in vivo or in vitro.

In one embodiment of the method of the above aspects, the immune effector cells to be genetically modified are present in vivo in a subject and the method comprises administering the particles to the subject.

In one embodiment of the method of the above aspects, the method is a method for treating or preventing cancer in a subject. In one embodiment, the cancer is solid cancer. In one embodiment, the cancer is associated with expression or elevated expression of a tumor antigen targeted by the T cell receptor. In one embodiment, the antigen concerned is NY-ESO-1, MAGE-A3, tyrosinase and KRAS, respectively, and the method of the present invention is useful in the treatment and/or prevention of a malignant disease.

In one embodiment of the method of the above aspects, the method further comprises administering to the subject an antigen targeted by the T cell receptor, a polynucleotide encoding the antigen, or a host cell genetically modified to express the antigen. In one embodiment, the polynucleotide encoding the antigen is RNA. In one embodiment, the host cell genetically modified to express the antigen comprises a polynucleotide encoding the antigen.

In one embodiment of the method of the above aspects, the immune effector cells genetically modified to express the T cell receptor comprise a polynucleotide encoding the T cell receptor. In one embodiment, the nucleic acid is RNA. In one embodiment, the nucleic acid is DNA.

In one embodiment of the method of the above aspects, the genetic modification is transient or stable.

In one embodiment of the method of the above aspects, the genetic modification takes place by a virus-based method, a transposon-based method, or a gene editing-based method. In one embodiment, the gene editing-based method involves CRISPR-based gene editing.

In one embodiment of the method of the above aspects, the particles are non-viral particles.

In one embodiment of the method of the above aspects, the particles are lipid-based and/or polymer-based particles.

In one embodiment of the method of the above aspects, the particles are nanoparticles.

In one embodiment of the method of the above aspects, the particles are functionalized with a targeting molecule on their surface.

In one embodiment of the method of the above aspects, the particles are functionalized with a targeting molecule by linking the targeting molecule to at least one particle-forming component.

In one embodiment of the method of the above aspects, the targeting molecule targets CD8, CD4 or CD3.

In one embodiment of the method of the above aspects, the immune effector cells are T cells.

In one embodiment of the method of the above aspects, the immune effector cells are CD4+ or CD8+ T cells.

The compositions and agents described herein are preferably capable of inducing or promoting a cellular response, preferably cytotoxic T cell activity, against a disease characterized by presentation of an antigen described herein with class I MHC, e.g., a malignant disease.

In one aspect, the invention provides the agents and compositions described herein for use in the methods of treatment described herein.

The treatments of malignant diseases described herein can be combined with surgical resection and/or radiation and/or traditional chemotherapy.

Other features and advantages of the instant invention will be apparent from the following detailed description and claims.

### BRIEF DESCRIPTION OF THE DRAWINGS

FIG. 1. Trial design and melanoma FixVac-mediated immune activation. A, Molecular structure of the four vaccine RNAs. 5′-cap, 5′- and 3′-untranslated regions (UTRs) and the poly(A) tail are optimized for stability and translational efficiency of the RNA in human dendritic cells. The signal peptide (SP), tetanus toxoid CD4⁺ epitopes P2 and P16 and the MHC class I trafficking domain (MITD) are fused to each TAA-encoding sequence for enhanced presentation on HLA class I and II molecules and immunogenicity. B, High level clinical trial design.

FIG. 2. TAA-specific T-cell immunity and clinical responses induced by melanoma FixVac. A, Ex vivo CD8⁺ T-cell responses of patient A2-009 measured after pulsing PBMCs with individual TAA PepMixes. PBMCs incubated with medium served as control. B, C, Kinetics of vaccine associated antigen-specific T cells determined by (B) multimer analysis or (C) intracellular cytokine staining (ICS) of T cells stimulated with single peptides or PepMixes.

FIG. 3. Discovery and characterization of NY-ESO-1-specific TCRs from post treatment PBMCs of patient A2-009. A, PBMC were stimulated with NY-ESO-1 PepMix and single IFNγ positive CD8⁺ T cells were sorted via flow cytometry for TCR cloning (Control; HIV-gag OLP pool). B, HLA restriction and epitope specificity of NY-ESO-1-TCRs was analyzed after co-culture of TCR-transfected CD8⁺ T cells with HLA-transfected K562 cells using IFNγ ELISPOT. C) The specific lysis of NY-ESO-1-positive (SK-MEL-37) and NY-ESO-1—negative (SK-MEL-28) melanoma cell lines by NY-ESO-1-TCR transfected T cells was assessed using xCELLigence cell index (CI) impedance measurements. Specific lysis of HLA-transfected melanoma cell lines was determined after 12 h co-culture (E:T=20:1). Bars represent the mean percent specific lysis+s.d. D, TRB chain frequencies of NY-ESO-1-specific TCRs.

FIG. 4. TAA-specific T-cell immunity in patient 53-02 with partial response under melanoma FixVac monotherapy. A, CT scans of lower and middle lobes of the right lung before (pre) and after starting (post) melanoma FixVac treatment. B, Size kinetics of multiple melanoma lesions of the patient under treatment as assessed by CT. Lesions that declined below a quantifiable size were plotted with a diameter of 0.1 mm. T; target lesions, NT; non-target lesions according to irRECISTv1.1; C, Kinetics of NY-ESO-1₉₆₋₁₀₄-specific Cw*0304-restricted CD8⁺ T cells analyzed by HLA multimer staining. D, Kinetics of the ex vivo frequency of NY-ESO-1 specific, cytokine secreting CD8⁺ T cells measured by intracellular cytokine staining (ICS) after stimulation with NY-ESO-1₉₆₋₁₀₄ peptide. E, (upper panel) Melanoma cell line killing by vaccine-induced CD8⁺ T cells from post treatment PBMCs. Specific killing of HLA-transfected melanoma cell lines by CD8⁺ T cells from short-term IVS cultures (E:T=20:1) was analyzed after 63 h co-culture. Bars represent the mean percent lysis+s.d. of technical triplicates. (lower panel) Frequencies of NY-ESO-1₉₆₋₁₀₄ multimer-specific CD8⁺ T cells after IVS of PBMCs from three different time points under treatment (−1 baseline, d22 after 3 vaccinations, d64 after 7 vaccinations).

FIG. 5. Discovery and characterization of a NY-ESO-196404-specific HLA-Cw*0304-restricted TCR. A, Sorting gate of multimer-positive CD8⁺ T cells for TCR cloning. Control, fluorescence minus one (FMO) sample. B, Recognition of peptide-pulsed HLA-Cw*0304-transfected K562 cells by NY-ESO-1-TCR-transfected CD8⁺ T cells in IFNγ ELISpot. Control; HIV-gag OLP pool, NY-ESO-1; NY-ESO-1 PepMix. C, Cytotoxicity of NY-ESO-1-TCR transfected CD8⁺ T cells after 24 h co-culture with HLA-transfected melanoma cell lines (E:T=50:1). Bars represent the mean % specific lysis+s.d. of technical triplicates. D, Kinetics of frequencies of the NY-ESO-1-specific TCR clonotype in TCR repertoire data obtained from pre- and post-vaccination PBMCs.

FIG. 6. Discovery and characterization of two NY-ESO-1₁₂₄₋₁₃₃-specific HLA-B*4001-restricted TCRs. A, PBMC were stimulated with NY-ESO-1 PepMix and single IFNγ positive CD8⁺ T cells were sorted via flow cytometry for TCR cloning (Control; HIV-gag OLP pool). B, C, HLA restriction and epitope specificity of NY-ESO-1-TCRs was analyzed after co-culture of TCR-transfected CD8⁺ T cells with HLA-transfected K562 cells using IFNγ ELISpot. D, Cytotoxicity of NY-ESO-1-specific TCRs identified in post vaccination samples of the patient. TCR-transfected CD8⁺ T cells were stimulated with HLA-transfected melanoma cell lines for 12 h at an effector to target ratio of 20:1. Bars represent the mean percent specific lysis+s.d. of technical triplicates. E, Kinetics of frequencies of the NY-ESO-1-specific TCR clonotypes in TCR repertoire data obtained from pre- and post-vaccination PBMCs.

FIG. 7. MAGE-A3-specific T-cell immunity in patient C2-28 with partial response under melanoma FixVac in combination with anti-PD1 and correlation of expression of TAAs with tumour mutational burden. A, Clinical response as % change in sum of target lesion size. B, De novo induced MAGE-A3-specific CD8+ T cells as measured by flow cytometry (lower panel: exemplary plots) in patient C2-28.

FIG. 8. Discovery and characterization of MAGE-A3₁₆₈₋₁₇₆-specific HLA-A*0101-restricted TCRs. A, Post treatment PBMCs were stained with HLA-A*0101/MAGE-A3₁₆₈₋₁₇₆ multimer and single multimer-positive CD8⁺ T cells were sorted by flowcytometry for TCR cloning. Control, fluorescence minus one (FMO) sample. B, C, Recognition of MAGE-A3-negative SK-MEL-29 (B) and MAGE-A3-positive SK-MEL-28 (C) melanoma cells by MAGE-A3₁₆₈₋₁₇₆-specific TCRs obtained from post treatment PBMCs of C2-28 using a bioluminescence based T-cell activation assay. Bars represent the mean luminescence value+s.d. of technical duplicates.

FIG. 9. MAGE-A3-specific T-cell immunity in patient C1-40 with partial response under melanoma FixVac in combination with anti-PD1. A, CT scans of the right middle and left lower lung lobes of patient C1-40 before (pre) and after start (post) of melanoma FixVac treatment. B, Kinetics of the ex vivo frequency of MAGE-A3168176-specific HLA-A*0101-restricted T cells analyzed by multimer staining. C, (upper panel) Melanoma cell line killing by vaccine-induced CD8⁺ T cells from post treatment PBMCs. Specific killing of HLA-transfected melanoma cell lines by CD8⁺ T cells from short-term IVS cultures of PBMCs harvested at different time points under treatment (E:T=8.5:1) was analyzed after 8 h co-culture. Bars represent the mean percent lysis+s.d. of technical triplicates (for day 36 only duplicates). (lower panel) Frequencies of MAGE-A3₁₆₈₋₁₇₆ multimer-specific CD8⁺ T cells after IVS.

FIG. 10. Discovery and characterization of a MAGE-A3₁₆₈₋₁₇₆-specific HLA-A*0101-restricted TCR from patient C1-40. A, CD8⁺ IVS cells from post treatment PBMCs (after 8 vaccinations) were stimulated with MAGE-A3₁₆₈₋₁₇₆ and single IFNγ positive CD8⁺ T cells were sorted via flow cytometry for TCR cloning. Control, SSX2₄₁₋₄₉. B, Recognition of HLA-A*0101-transfected K562 cells by MAGE-A3₁₆₈₋₁₇₆-specific TCRs obtained from post treatment PBMCs of C1-40 using a bioluminescence based T-cell activation assay. Bars represent the mean luminescence value+s.d. of technical duplicates.

FIG. 11. TAA-specific T-cell immunity in patient A2-10 with partial response. A, CT scans of an inguinal lymph node metastasis of patient A2-10 obtained before and after vaccination start. B, CD4⁺ T-cell responses of the patient after in vitro stimulation with TAA-encoding RNAs pre-vaccination and after 8 vaccinations. Cells were re-stimulated in an IFNγ-ELISpot assay by co-culture with autologous DCs transfected with RNA (TAA or luciferase as control), pulsed with TAA (PepMix) or unpulsed DCs (no peptide). Bars represent median+range of spot counts from triplicates.

FIG. 12. Characterization of HLA class II restricted TAA-specific T-cell responses in patient A2-10 with partial response. A, CD4⁺ T cells from IVS cultures were restimulated with PepMix-pulsed DCs and sorted via flow cytometry for TCR cloning (Control, HIV-gag OLP pool). B, C, Determination of HLA restriction (B) and epitope specificity (C) using TCR-transfected CD4⁺ T cells and HLA-transfected K562 cells by IFNγ ELISpot. D, Kinetics of TCR clonotype frequencies in peripheral blood by ex vivo TCR repertoire analysis.

FIG. 13. Discovery and characterization of a HLA-A*02:01-restricted, NY-ESO-1-specific TCR from a checkpoint inhibitor treated (CIT) NSCLC-patient. A, CD8⁺ T cells obtained post CIT treatment from NSCLC patient EL28 were stimulated with NY-ESO-1 IVT-RNA transfected iDCs and single T cells were sorted based on the expression of activation marker CD137 and/or IFNγ via flow cytometry for TCR cloning. Cells were gated on single, viable CD8⁺ T lymphocytes. Control, iDCs transfected with RNA encoding an irrelevant antigen (eGFP); B, HLA restriction and target specificity of NY-ESO-1-TCR was analyzed after co-culture of TCR_{CD8}-EL28-NYE #2-transfected CD8⁺ T cells with HLA-transfected and peptide pulsed K562 cells using IFNγ ELISPOT. Controls, CD8⁺ T cells transfected without RNA; effectors only (Eft); positive control, Staphylococcal enterotoxin B (SEB); C, Epitope specificity of TCR_{CD8}-EL28-NYE #2 was analyzed after co-culture of TCR-transfected Jurkat cells and peptide-pulsed HLA-transfected K562 target cells using a bioluminescence based T-cell activation assay. Bars represent the mean luminescence value+s.d. of technical triplicates. D, Recognition of endogenously expressed NY-ESO-1 was determined with TCR_{CD8}-EL28-NYE #2-transfected CD8⁺ T cells and NSCLC cell line LCLC-103H using IFNγ ELISPOT. Positive control, Phytohemagglutinin-L (PHA-L); E, Specific lysis of endogenously NY-ESO-1 expressing NSCLC cells by TCR_{CD8}-EL28-NYE #2 expressing CD8⁺ T cells. TCR-transfected T cells were co-cultured with HLA-A*0201-transfected LCLC-103H cells (E:T=30:1) in the presence (w peptide) or absence (w/o peptide) of NY-ESO-1₁₅₇₋₁₆₅ for 24 h. Control, T cells transfected without RNA. Bars represent the mean % specific lysis+s.d. of technical triplicates. F, Lysis of endogenously NY-ESO-1 expressing melanoma cell line mediated by TCR_{CD8}-EL28-NYE #2. TCR-transfected CD8⁺ T cells were co-cultured with NY-ESO-1 positive (SK-Mel-37) and negative (SK-Mel-28) melanoma cell lines (E:T=for 24 h. Bars represent the mean % specific lysis+s.d. of technical triplicates G, TRB chain frequency of NY-ESO-1-specific clonotype TCR_{CD8}-EL28-NYE #2 in the PBMC of the patient pre and post CIT treatment.

FIG. 14. Discovery and characterization of four HLA-A*01:01-restricted KRAS-Q61-H-specific TCRs from a NSCLC-patient with primary. A, CD8⁺ T cells were stimulated with iDCs transfected with Multitope #7 IVT-RNA encoding six neo epitopes including KRAS-Q61H48-74. Single T cells were sorted by flow cytometry based on expression of activation marker CD137 and or IFNγ for TCR cloning. Control, iDCs transfected with IVT-RNA encoding the irrelevant antigen eGFP; B, HLA restriction and specificity for Multitope #7 (including KRAS-Q61H48-74) was determined after co-culture of TCR-transfected CD8⁺ T cells with K562 cells transfected with HLA-A*0101 and Multitope #7 RNA using IFNγ ELISPOT. C, Epitope specificity of KRAS-Q61H₅₅₋₆₄ TCRs. TCR-transfected CD8⁺ T cells were stimulated with peptide-pulsed HLA-A*0101-transfected K562 target cells using IFNγ ELISPOT. Controls, irrelevant peptides derived from HIV-gag; effectors alone (Eff); unspecific stimulation with Staphylococcal enterotoxin B (SEB); D, Functional avidity of HLA-A*01:01-restricted KRAS-Q61H66-64-specific TCRs. TCR-transfected CD8⁺ T cells co-cultured with HLA-A*0101-transfected K562 cells pulsed with titrated amounts of KRAS-Q61H₅₅₋₆₄ peptide were analysed by IFNγ ELISPOT. E, Specific lysis of endogenously KRAS-Q61H expressing NSCLC cells mediated by TCR_{CD8}-EL8-LM7 #1. TCR-transfected CD8⁺ FT cells were co-cultured with HLA-A*0101-transfected NSCLC cell line NCI-H-460 in the presence (w peptide) or absence (w/o peptide) of KRAS-Q61H₅₅₋₆₄ (E:T=30:1) for 24 h. Control, T cells transfected without RNA; Bars represent the mean % specific lysis+s.d. of technical triplicates.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

### Definitions

A reference to SEQ ID NOs: 39 to 44 is to be understood so as to refer individually to each of SEQ ID NOs: 39, 40, 41, 42, 43 and 44.

Terms such as “reduce”, “decrease”, “inhibit” or “impair” as used herein relate to an overall decrease or the ability to cause an overall decrease, preferably of 5% or greater, 10% or greater, 20% or greater, more preferably of 50% or greater, and most preferably of 75% or greater, in the level, e.g. in the level of binding.

Terms such as “increase”, “enhance” or “exceed” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, or even more.

The term “plurality” with reference to an object refers to a population of a certain number of said object. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 151 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and cytokines.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” or “variant protein” or “variant polypeptide” herein is meant a protein that differs from a wild type protein by virtue of at least one amino acid modification. The parent polypeptide may be a naturally occurring or wild type (WT) polypeptide, or may be a modified version of a wild type polypeptide. Preferably, the variant polypeptide has at least one amino acid modification compared to the parent polypeptide, e.g. from 1 to about 20 amino acid modifications, and preferably from 1 to about 10 or from 1 to about 5 amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild type polypeptide, or a variant or engineered version of a wild type polypeptide.

By “wild type” or “WT” or “native” herein is meant an amino acid sequence that is found in nature, including allelic variations. A wild type protein or polypeptide has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all splice variants, posttranslationally modified variants, conformations, isoforms and species homologs, in particular those which are naturally expressed by cells. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigen peptides, one particular function is one or more immunostimulating activities displayed by the amino acid sequence from which the fragment or variant is derived and/or binding to the molecules such as MHC and receptor(s) the amino acid sequence from which the fragment or variant is derived binds to. With respect to antigen receptors such as T cell receptors, one particular function is one or more immunostimulating activities displayed by the amino acid sequence from which the fragment or variant is derived and/or binding to the molecules such as MHC and epitope the amino acid sequence from which the fragment or variant is derived binds to. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In one embodiment, the modifications in the amino acid sequence of the parent molecule or sequence do not significantly affect or alter the binding characteristics of the molecule or sequence. In different embodiments, binding of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of a kit may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

By the term “specifically binds”, as used herein, is meant a molecule such as a TCR which recognizes a specific target such as an antigen or antigen peptide, but does not substantially recognize or bind other molecules in a sample or in a subject. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “genetic modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can occur if the nucleic acid introduced in the transfection process is integrated into the nuclear genome and can be achieved, for example, by using virus-based systems or transposon-based systems for transfection. Generally, cells that are genetically modified to express an antigen receptor such as a T cell receptor are stably transfected with nucleic acid encoding the antigen receptor, while, generally, nucleic acid encoding antigen is transiently transfected into cells.

### Immune Effector Cells

The cells used in connection with the present invention and into which nucleic acids (DNA or RNA) encoding antigen receptors, in particular T cell receptors, may be introduced include, in particular, immune effector cells such as cells with lytic potential, in particular lymphoid cells, and are preferably T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cells used in connection with the present invention will preferably be autologous cells, although heterologous cells or allogenic cells can be used.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present invention relates to a cell which exerts effector functions during an immune reaction. An “immune effector cell” in one embodiment is capable of binding an antigen such as an antigen presented in the context of MHC on a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells, most preferably CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen with some degree of specificity, in particular if presented in the context of MHC on the surface of diseased cells such as cancer cells. Preferably, said recognition enables the cell that recognizes an antigen to be responsive or reactive. If the cell is a helper T cell (CD4⁺ T cell) such responsiveness or reactivity may involve the release of cytokines and/or the activation of CD8⁺ lymphocytes (CTLs) and/or B cells. If the cell is a CTL such responsiveness or reactivity may involve the elimination of cells, i.e., cells characterized by expression of an antigen, for example, via apoptosis or perforin-mediated cell lysis. According to the invention, CTL responsiveness may include sustained calcium flux, cell division, production of cytokines such as IFN-γ and TNF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

In one embodiment, the genetically modified immune effector cells are TCR-expressing immune effector cells. The immune effector cells to be used according to the invention may express an endogenous antigen receptor such as T cell receptor or B cell receptor or may lack expression of an endogenous antigen receptor.

A “lymphoid cell” is a cell which, optionally after suitable modification, e.g. after transfer of an antigen receptor such as a TCR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In one embodiment, the lymphoid cell lacks endogenous expression of a T cell receptor.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45R0 isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4⁺ or CD8⁺ and usually express CD45RO.

According to the invention, the term “T cell” also includes a cell which can mature into a T cell with suitable stimulation.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

The structure of the T cell receptor is very similar to immunoglobulin Fab fragments, which are regions defined as the combined light and heavy chain of an antibody arm. Each chain of the TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end.

A TCR described herein may have a naturally occurring, non-naturally occurring or engineered TCR format. A TCR described herein may be an alpha-beta heterodimer, preferably having an alpha chain constant domain sequence and a beta chain constant domain sequence. The alpha and beta chain constant domain sequences may be modified by truncation or substitution, e.g., to delete native disulphide bonds. In one embodiment, a TCR described herein may be in single chain format, e.g., of the type Vα-L-Vβ, Vβ-L-Vα, Vα-Ca-L-Vβ, Vα-L-Vβ-Cβ, wherein Vα and Vβ are TCR α and β variable regions, respectively, Cα and Cβ are TCR α and β constant regions, respectively, and L is a linker sequence. A TCR described herein may be associated with or linked to other moieties such as a detectable label, a therapeutic agent or a PK modifying moiety. A TCR described herein may be a TCR anti-CD3 fusion comprising a TCR and an anti-CD3 antibody or antibody fragment. The anti-CD3 antibody or antibody fragment may be covalently linked to the C- or N-terminus of the alpha or beta chain of the TCR.

According to the invention, the term “variable region of a T cell receptor” relates to the variable domains of the TCR chains. With respect to T cell receptor sequences described herein (e.g., SEQ ID NOs: 5-38, and 91-100), the term “variant” as used herein in one embodiment refers to a fragment of the T cell receptor sequences just including the variable region or domain, i.e., to a fragment of the T cell receptor sequences not including constant region sequence portions.

The variable domain of both the TCR α-chain and β-chain have three hypervariable or complementarity determining regions (CDRs), whereas the variable region of the β-chain has an additional area of hypervariability (HV4) that does not normally contact antigen and therefore is not considered a CDR. CDR3 is the main CDR responsible for recognizing processed antigen, although CDR1 of the α-chain has also been shown to interact with the N-terminal part of the antigenic peptide, whereas CDR1 of the β-chain interacts with the C-terminal part of the peptide. CDR2 is thought to recognize the MHC. HV4 of the β-chain is not thought to participate in antigen recognition, but has been shown to interact with superantigens.

The phrase “T cell receptor chain comprising a CDR sequence of a T cell receptor chain” relates to a T cell receptor chain comprising as the respective CDR the CDR of said other T cell receptor chain.

According to the invention, the term “at least one of the CDR sequences” refers, in particular, to one or more of the complementarity-determining regions (CDRs), preferably at least the CDR3 region, of the α-chain and/or β-chain of a T cell receptor. Preferably, the term “at least one of the CDR sequences” means at least the CDR3 sequence. In one embodiment, the term “CDR sequences of a T cell receptor chain” preferably relates to CDR1, CDR2 and CDR3 of the α-chain and/or β-chain of a T cell receptor. In one embodiment said one or more of the complementarity-determining regions (CDRs) are selected from a set of complementarity-determining regions CDR1, CDR2 and CDR3. In a particularly preferred embodiment, the term “at least one of the CDR sequences” refers to the complementarity-determining regions CDR1, CDR2 and CDR3 of the α-chain and/or β-chain of a T cell receptor.

In one embodiment a variable domain of a TCR comprising one or more CDRs, a set of CDRs or a combination of sets of CDRs as described herein comprises said CDRs together with their intervening framework regions.

In one embodiment a TCR α-chain which is optionally part of a TCR further comprising a TCR β-chain comprises a variable domain comprising at least one, preferably two, more preferably all three of the CDR sequences of a TCR α-chain variable domain described herein.

In one embodiment a TCR β-chain which is optionally part of a TCR further comprising a TCR α-chain comprises a variable domain comprising at least one, preferably two, more preferably all three of the CDR sequences of a TCR β-chain variable domain described herein.

The constant domain of the TCR domain consists of short connecting sequences in which a cysteine residue forms disulfide bonds, which forms a link between the two chains.

Nucleic acid such as RNA encoding T cell receptor (TCR) chains may be introduced into immune effector cells such as T cells or other cells with lytic potential. In a suitable embodiment, the TCR α- and β-chains are cloned out from an antigen-specific T cell or T cell line and used for adoptive T cell therapy. The present invention provides T cell receptors specific for an antigen or antigen peptide (epitope) disclosed herein. In general, this aspect of the invention relates to T cell receptors, which recognize or bind antigen peptides presented in the context of MHC. The nucleic acids encoding α- and β-chains of a T cell receptor, e.g. a T cell receptor provided according to the present invention, may be contained on separate nucleic acid molecules such as expression vectors or alternatively, on a single nucleic acid molecule. Accordingly, the term “nucleic acid encoding a T cell receptor” relates to nucleic acid molecules encoding the T cell receptor chains on the same or preferably on different nucleic acid molecules.

The term “immune effector cell reactive with a peptide” relates to an immune effector cell which when it recognizes the peptide, in particular if presented in the context of MHC molecules such as on the surface of antigen presenting cells or diseased cells such as malignant cells, exerts effector functions of immune effector cells as described herein.

The term “T cell receptor reactive with a peptide” relates to a T cell receptor which when present on an immune effector cell recognizes the peptide, in particular if presented in the context of MHC molecules such as on the surface of antigen presenting cells or diseased cells such as malignant cells, such that the immune effector cell exerts effector functions of immune effector cells as described herein.

The term “antigen-reactive cell” or similar terms relate to a cell, which recognizes an antigen if presented in the context of MHC molecules such as on the surface of antigen presenting cells or diseased cells such as malignant cells and exerts effector functions of immune effector cells as described herein.

The term “antigen-specific cell” or similar terms relates to a cell, which, in particular when provided with an antigen-specific T cell receptor, recognizes the antigen if presented in the context of MHC molecules such as on the surface of antigen presenting cells or diseased cells such as malignant cells and preferably exerts effector functions of immune effector cells as described herein. T cells and other lymphoid cells are considered to be specific for antigen, if the cells kill target cells expressing an antigen and/or presenting an antigen peptide or secrete cytokines after binding to such target cells via an antigen expressed or an antigenic peptide presented by such target cells.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

As used herein, the term “NK cell” or “Natural Killer cell” refers to a subset of peripheral blood lymphocytes defined by the expression of CD56 or CD16 and the absence of the T cell receptor. As provided herein, the NK cell can also be differentiated from a stem cell or progenitor cell.

### Genetic Modification to Express Antigen Receptors

Cells described herein such as immune effector cells may be genetically modified ex vivo/in vitro or in vivo in a subject being treated to express an antigen receptor such as a T cell receptor (TCR) binding antigen or a procession product thereof, in particular when presented by a target cell, e.g., an antigen presenting cell or a diseased cell. Cells may naturally express an antigen receptor or be modified (e.g., ex vivo/in vitro or in vivo in a subject to be treated) to express an antigen receptor. In one embodiment, modification to express an antigen receptor takes place ex vivo/in vitro. Subsequently, modified cells may be administered to a patient. In one embodiment, modification to express an antigen receptor takes place in vivo. The cells may be endogenous cells of the patient or may have been administered to a patient.

A variety of methods may be used to introduce antigen receptors such as TCR constructs into cells such as T cells to produce cells genetically modified to express the antigen receptors. Such methods include non-viral-based DNA transfection, non-viral-based RNA transfection, e.g., mRNA transfection, transposon-based systems, and viral-based systems. Non-viral-based DNA transfection has low risk of insertional mutagenesis. Transposon-based systems can integrate transgenes more efficiently than plasmids that do not contain an integrating element. Viral-based systems include the use of γ-retroviruses and lentiviral vectors. γ-Retroviruses are relatively easy to produce, efficiently and permanently transduce T cells, and have preliminarily proven safe from an integration standpoint in primary human T cells. Lentiviral vectors also efficiently and permanently transduce T cells but are more expensive to manufacture. They are also potentially safer than retrovirus based systems.

Particles as described herein that may be functionalized with a targeting moiety as described herein for specific targeting of immune effector cells, in particular CD8⁺ T cells, may be used ex vivo/in vitro or in vivo for delivering nucleic acid encoding antigen receptors such as T cell receptors to immune effector cells such as T cells to produce cells genetically modified to express the antigen receptors.

In one embodiment of all aspects of the invention, T cells or T cell progenitors are transfected either ex vivo or in vivo with nucleic acid encoding the antigen receptor. In one embodiment, a combination of ex vivo and in vivo transfection may be used. In one embodiment of all aspects of the invention, the T cells or T cell progenitors are from the subject to be treated. In one embodiment of all aspects of the invention, the T cells or T cell progenitors are from a subject which is different to the subject to be treated.

In one aspect of the invention, genetically modified T cells may be produced in vivo, and therefore nearly instantaneously, using particles such as nanoparticles described herein targeted to T cells. For example, lipid and/or polymer-based nanoparticles may be coupled to CD3-, CD8, or CD4-specific targeting moieties for binding to CD3, CD8, or CD4 on T cells or T cell subpopulations, respectively. Upon binding to T cells, these particles are endocytosed. Their contents, for example nucleic acid encoding antigen receptor, e.g., plasmid DNA encoding an anti-tumor antigen TCR, may be directed to the T cell nucleus due to, for example, the inclusion of peptides containing microtubule-associated sequences (MTAS) and nuclear localization signals (NLSs). The inclusion of transposons flanking the nucleic acid encoding antigen receptor, e.g., the TCR gene expression cassette, and a separate nucleic acid, e.g., plasmid, encoding a hyperactive transposase, may allow for the efficient integration of the nucleic acid encoding antigen receptor, e.g., the TCR vector, into chromosomes.

Another possibility is to use a CRISPR/Cas9 method, e.g. prime editing as described in Anzalone et al. (2019) Nature 576(7785):149-157, to deliberately place an antigen receptor coding sequence such as a TCR coding sequence at a specific locus. For example, existing T cell receptors (TCR) may be knocked out, while knocking in the antigen receptor and placing it under the dynamic regulatory control of the endogenous promoter that would otherwise moderate expression of an endogenous TCR.

Accordingly, besides nucleic acid encoding an antigen receptor the particles described herein may also deliver as cargo gene editing tools like CRISPR/Cas9 (or related) or transposon systems like sleeping beauty or piggy bac. Such tools (e.g. transposase, gene editing tools like CRISPR/Cas9) for genomic integration/editing may be delivered as protein or coding nucleic acid (DNA or RNA). Nevertheless, also delivery of mRNA or self-amplifying RNA are options to induce transient expression of antigen receptors like T cell receptors (TCR).

In one embodiment of all aspects of the invention, the cells genetically modified to express an antigen receptor are stably or transiently transfected with nucleic acid encoding the antigen receptor. Thus, the nucleic acid encoding the antigen receptor is integrated or not integrated into the genome of the cells.

In one embodiment of all aspects of the invention, the cells genetically modified to express an antigen receptor are inactivated for expression of an endogenous T cell receptor and/or endogenous HLA.

In one embodiment of all aspects of the invention, the cells described herein may be autologous, allogeneic or syngeneic to the subject to be treated. In one embodiment, the present disclosure envisions the removal of cells from a patient and the subsequent re-delivery of the cells to the patient. In one embodiment, the present disclosure does not envision the removal of cells from a patient. In the latter case all steps of genetic modification of cells are performed in vivo.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

### Nucleic Acid Containing Particles

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material such as DOTAP, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and the cationic polymer combine together with the nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof.

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features,

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein, e.g. generated by the processes described herein, exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included. Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

Other methods having organic solvent free characteristics may also be used according to the present disclosure for preparing a colloid.

LNPs typically consist of four components: ionizable cationic lipids, phospholipids, cholesterol, and polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI). Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances. In one embodiment, the cationic or cationically ionizable lipid or lipid-like material comprises a head group which includes at least one nitrogen atom (N) which is positive charged or capable of being protonated. Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 245′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLin DMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-34(2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200). Preferred are DOTAP, DODMA, DOTMA, DODAC, and DOSPA. In specific embodiments, the at least one cationic lipid is DOTAP.

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid. In an exemplary embodiment, the cationic lipid is DOTAP and the additional lipid is DSPC or DSPC and cholesterol.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about mol %, from about 0 mol % to about 70 mol %, from about 0 mol % to about 60 mol %, or from about mol % to about 50 mol %, of the total lipid present in the particle.

### Targeting Molecules

One or more of the particle-forming components described herein such as polymers, lipids and/or lipid-like materials may comprise or may be functionalized with one or more targeting molecules that will direct the particle to immune effector cells, in particular T cells such as CD8⁺ T cells. The targeting molecules may be conjugated, in particular covalently or non-covalently bound to or linked to, any particle forming component such as a lipid, lipid-like material or polymer. Targeting molecules may, when fused to nucleic acid particle components such as lipids or proteins, specifically bind to CD8 exhibiting increased transfection of CD8+ T cells in vitro and in vivo as compared to non-targeting molecule-functionalized particles. Targeting molecules include, but are not limited to, antibodies, antibody fragments, and ankyrin repeat proteins.

CD8 is a primary marker of the cytotoxic subset of T lymphocytes. CD8 is a type-I single pass transmembrane protein expressed as disulfide-linked homo- or heterodimeric molecule on the surface of immune cells. The CD8 heterodimer consists of the CD8a and CD813 chain and is only expressed on the surface of immature CD4+CD8+ double-positive thymocytes and mature peripheral cytotoxic αβ T cells. The homodimer consists of two CD8α chains and exhibits expression on a much broader range of immune cells. In addition to classic cytotoxic αβ T cells and thymocytes, it is found on natural killer T (NKT) cells, a subset of dendritic cells (DC), and natural killer (NK) cell subpopulations. Both CD8αβ and CD8αα can mediate MHC-I binding; still, the heterodimeric form is more prevalent on the surface of MHC-I-restricted cytotoxic T cells. Of note, CD8ββ-homodimers do not occur naturally.

The term “antibody” includes an immunoglobulin comprising at least two heavy (H) chains and two light (L) chains inter-connected by disulfide bonds. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An antibody binds, preferably specifically binds with an antigen. Antibodies can be intact immunoglobulins derived from natural sources or from recombinant sources and can be immunoreactive portions or fragments of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab′)₂, as well as single chain antibodies and humanized antibodies (Harlow et al., 1999, In: Using Antibodies: A Laboratory Manual, Cold Spring Harbor Laboratory Press, NY; Harlow et al., 1989, in: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and typically comprises the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain”, as used herein, refers to the larger of the two types of polypeptide chains present in antibody molecules in their naturally occurring conformations.

An “antibody light chain”, as used herein, refers to the smaller of the two types of polypeptide chains present in antibody molecules in their naturally occurring conformations, K and A light chains refer to the two major antibody light chain isotypes.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, nucleic acid such as nucleic acid encoding an antigen receptor or nucleic acid encoding a vaccine antigen is expressed in cells of the subject treated to provide the antigen receptor or vaccine antigen. In one embodiment of all aspects of the invention, the nucleic acid is transiently expressed in cells of the subject. Thus, in one embodiment, the nucleic acid is not integrated into the genome of the cells. In one embodiment of all aspects of the invention, the nucleic acid is RNA, preferably in vitro transcribed RNA. In one embodiment of all aspects of the invention, expression of the antigen receptor is at the cell surface. In one embodiment of all aspects of the invention, the vaccine antigen is expressed and presented in the context of MHC.

In one embodiment of all aspects of the invention, the nucleic acid encoding the vaccine antigen is expressed in cells such as antigen presenting cells of the subject treated to provide the vaccine antigen for binding by the immune effector cells expressing an antigen receptor, said binding resulting in stimulation, priming and/or expansion of the immune effector cells expressing an antigen receptor.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, the RNA is replicon RNA or simply “a replicon”, in particular self-replicating RNA. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In one embodiment, the RNA may have modified ribonucleotides. Examples of modified ribonucleotides include, without limitation, 5-methylcytidine, pseudouridine and/or 1-methyl-pseudouridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′ to 5′ triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

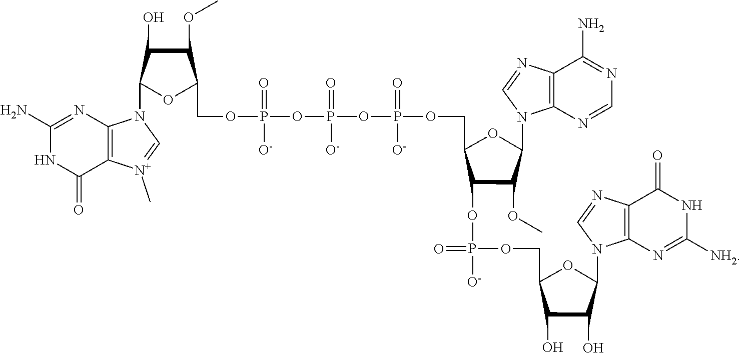

Below is an exemplary Cap1 RNA, which comprises RNA and m₂^{7,3′O}G(5′)ppp(5′)m^{2′-O}ApG:

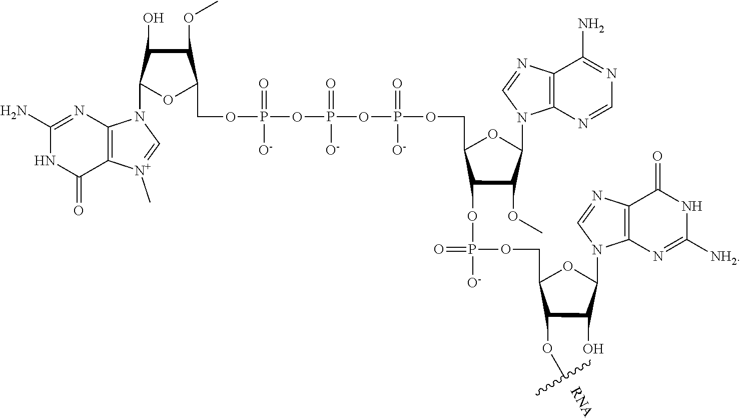

Below is another exemplary Cap1 RNA (no cap analog):

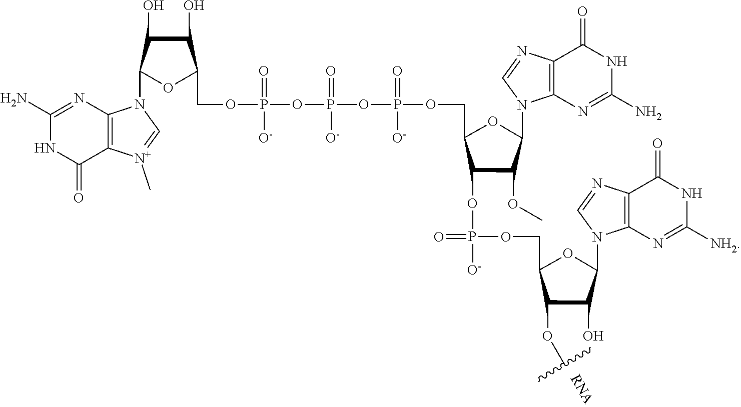

In some embodiments, the RNA is modified with “Cap0” structures using, in one embodiment, the cap analog anti-reverse cap (ARCA Cap (m₂^{7,3′O}G(5′)ppp(5′)G)) with the structure:

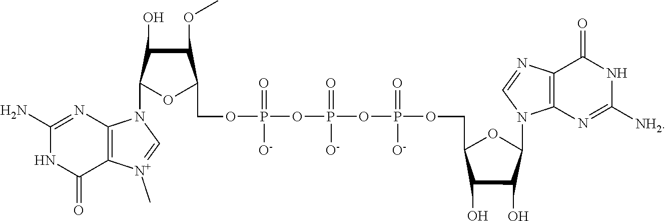

Below is an exemplary Cap0 RNA comprising RNA and m₂^{7,3′O}(5′)ppp(5′)G:

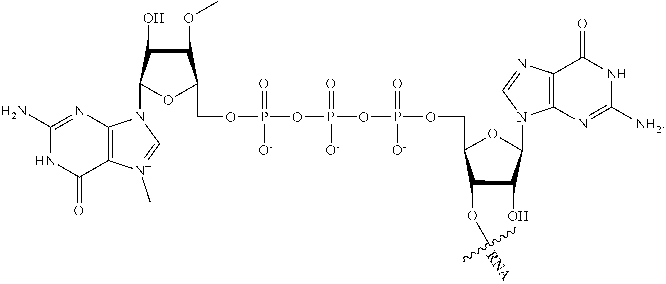

In some embodiments, the “Cap0” structures are generated using the cap analog Beta-S-ARCA (m₂^{7,2′O}(5′)ppSp(5′)G) with the structure:

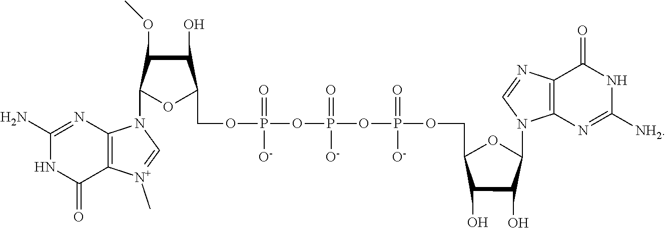

Below is an exemplary Cap0 RNA comprising Beta-S-ARCA (m₂^{7,2′O}G(5′)ppSp(5′)G) and RNA:

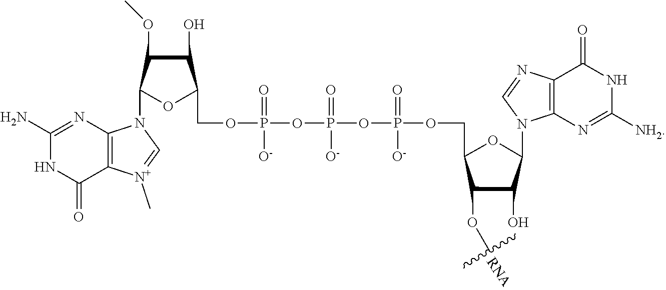

A particularly preferred Cap comprises the 5′-cap m₂^{7,2′O}G(5′)ppSp(5′)G.

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) tail. Thus, the 3′-UTR is upstream of the poly(A) sequence (if present), e.g. directly adjacent to the poly(A) sequence.

In some embodiments, the RNA according to the present disclosure comprises a 3′-poly(A) sequence. As used herein, the term “poly-A tail” or “poly-A sequence” refers to an uninterrupted or interrupted sequence of adenylate residues which is typically located at the 3′-end of an RNA molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides.

According to the disclosure, vaccine antigen is preferably administered as single-stranded, 5′-capped mRNA that is translated into the respective protein upon entering antigen-presenting cells (APCs). Preferably, the RNA contains structural elements optimized for maximal efficacy of the RNA with respect to stability and translational efficiency (5′-cap, 5′-UTR, 3′-UTR, poly(A)-tail).

In one embodiment, beta-S-ARCA(D1) is utilized as specific capping structure at the 5′-end of the RNA. In one embodiment, the 5′-UTR sequence is derived from the human alpha-globin mRNA. In one embodiment, two re-iterated 3′-UTRs derived from the human beta-globin mRNA are placed between the coding sequence and the poly(A)-tail to assure higher maximum protein levels and prolonged persistence of the mRNA. Alternatively, the 3′-UTR may be a combination of two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see WO 2017/060314, herein incorporated by reference). In one embodiment, a poly(A)-tail measuring 110 nucleotides in length, consisting of a stretch of 30 adenosine residues, followed by a 10 nucleotide linker sequence and another 70 adenosine residues is used. This poly(A)-tail sequence was designed to enhance RNA stability and translational efficiency in dendritic cells.

The RNA is preferably administered as lipoplex particles, preferably comprising DOTMA and DOPE, as further described below. Such particles are preferably administered by systemic administration, in particular by intravenous administration.

In the context of the present disclosure, the term “transcription” relates to a process, wherein the genetic code in a DNA sequence is transcribed into RNA. Subsequently, the RNA may be translated into peptide or protein.

With respect to RNA, the term “expression” or “translation” relates to the process in the ribosomes of a cell by which a strand of mRNA directs the assembly of a sequence of amino acids to make a peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm and/or in the nucleus), may secrete the encoded peptide or protein, or may express it on the surface.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence. Expression can be transient or stable. According to the invention, the term expression also includes an “aberrant expression” or “abnormal expression”.

As used herein, the terms “linked,” “fused”, or “fusion” are used interchangeably. These terms refer to the joining together of two or more elements or components or domains.

The term “cell” includes any viable cell, i.e. a living cell capable of carrying out its normal metabolic functions. In one embodiment, said term relates to any cell which can be transformed or transfected with an exogenous nucleic acid. The term “cell” includes according to the invention prokaryotic cells (e.g., E. coli) or eukaryotic cells (e.g., dendritic cells, B cells, CHO cells, COS cells, K562 cells, HEK293 cells, HELA cells, yeast cells, and insect cells). Mammalian cells are particularly preferred, such as cells from humans, mice, hamsters, pigs, goats, and primates. The cells may be derived from a large number of tissue types and include primary cells and cell lines. In certain embodiments, the cell is an antigen-presenting cell, in particular a dendritic cell, a monocyte, or macrophage, or an immune effector cell, in particular a T cell such as a cytotoxic T cell. A cell may be a recombinant cell and may comprise a nucleic acid, in particular a nucleic acid encoding a peptide or protein, which may have been delivered to the cell, e.g., by transfection. The cell may secrete the encoded peptide or protein, may express it on the surface and preferably may additionally express an MHC molecule which binds to said peptide or protein or a procession product thereof. In one embodiment, the cell expresses the MHC molecule endogenously. In a further embodiment, the cell expresses the MHC molecule and/or the peptide or protein or the procession product thereof in a recombinant manner. The cell is preferably nonproliferative.

### Cytokines

The methods described herein may comprise providing to a subject one or more cytokines, e.g., by administering to the subject the one or more cytokines, a polynucleotide encoding the one or more cytokines or a host cell expressing the one or more cytokines.

The term “cytokine” as used herein includes naturally occurring cytokines and functional variants thereof (including fragments of the naturally occurring cytokines and variants thereof). One particularly preferred cytokine is IL2.

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

### IL2

Interleukin-2 (IL2) is a cytokine that induces proliferation of antigen-activated T cells and stimulates natural killer (NK) cells. The biological activity of IL2 is mediated through a multi-subunit IL2 receptor complex (IL2R) of three polypeptide subunits that span the cell membrane: p55 (IL2Rα, the alpha subunit, also known as CD25 in humans), p75 (IL2Rβ, the beta subunit, also known as CD122 in humans) and p64 (IL2Rγ, the gamma subunit, also known as CD132 in humans). T cell response to IL2 depends on a variety of factors, including: (1) the concentration of IL2; (2) the number of IL2R molecules on the cell surface; and (3) the number of IL2R occupied by IL2 (i.e., the affinity of the binding interaction between IL2 and IL2R (Smith, “Cell Growth Signal Transduction is Quantal” In Receptor Activation by Antigens, Cytokines, Hormones, and Growth Factors 766:263-271, 1995)). The IL2:1L2R complex is internalized upon ligand binding and the different components undergo differential sorting. When administered as an intravenous (i.v.) bolus, IL2 has a rapid systemic clearance (an initial clearance phase with a half-life of 12.9 minutes followed by a slower clearance phase with a half-life of 85 minutes) (Konrad et al., Cancer Res. 50:2009-2017, 1990).

In eukaryotic cells human IL2 is synthesized as a precursor polypeptide of 153 amino acids, from which 20 amino acids are removed to generate mature secreted IL2. Recombinant human IL2 has been produced in E. coli, in insect cells and in mammalian COS cells.

According to the disclosure, IL2 (optionally as a portion of extended-PK IL2) may be naturally occurring IL2 or a fragment or variant thereof. IL2 may be human IL2 and may be derived from any vertebrate, especially any mammal.

### Extended-PK Group

Cytokine polypeptides described herein can be prepared as fusion or chimeric polypeptides that include a cytokine portion and a heterologous polypeptide (i.e., a polypeptide that is not a cytokine or a variant thereof). The resulting molecule, hereafter referred to as “extended-pharmacokinetic (PK) cytokine,” has a prolonged circulation half-life relative to free cytokine. The prolonged circulation half-life of extended-PK cytokine permits in vivo serum cytokine concentrations to be maintained within a therapeutic range, potentially leading to the enhanced activation of many types of immune cells, including T cells. Because of its favorable pharmacokinetic profile, extended-PK cytokine can be dosed less frequently and for longer periods of time when compared with unmodified cytokine.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK cytokine such as an extended-PK interleukin (IL) suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

The cytokine may be fused to an extended-PK group, which increases circulation half-life. Non-limiting examples of extended-PK groups are described infra. It should be understood that other PK groups that increase the circulation half-life of cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK cytokine” refers to a cytokine moiety in combination with an extended-PK group. In one embodiment, the extended-PK cytokine is a fusion protein in which a cytokine moiety is linked or fused to an extended-PK group. As used herein, an “extended-PK IL” refers to an interleukin (IL) moiety (including an IL variant moiety) in combination with an extended-PK group. In one embodiment, the extended-PK IL is a fusion protein in which an IL moiety is linked or fused to an extended-PK group. An exemplary fusion protein is an HSA/IL2 fusion in which an IL2 moiety is fused with HSA.

In certain embodiments, the serum half-life of an extended-PK cytokine is increased relative to the cytokine alone (i.e., the cytokine not fused to an extended-PK group). In certain embodiments, the serum half-life of the extended-PK cytokine is at least 20, 40, 60, 80, 100, 120, 150, 180, 200, 400, 600, 800, or 1000% longer relative to the serum half-life of the cytokine alone. In certain embodiments, the serum half-life of the extended-PK cytokine is at least 1.5-fold, 2-fold, 2.5-fold, 3-fold, 3.5 fold, 4-fold, 4.5-fold, 5-fold, 6-fold, 7-fold, 8-fold, 10-fold, 12-fold, β-fold, 15-fold, 17-fold, 20-fold, 22-fold, 25-fold, 27-fold, 30-fold, 40-fold, or 50-fold greater than the serum half-life of the cytokine alone. In certain embodiments, the serum half-life of the extended-PK cytokine is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular IL2 (or variant thereof). The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, mouse, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain aspects, the extended-PK cytokine, suitable for use according to the disclosure, can employ one or more peptide linkers. As used herein, the term “peptide linker” refers to a peptide or polypeptide sequence which connects two or more domains (e.g., the extended-PK moiety and an IL moiety such as IL2) in a linear amino acid sequence of a polypeptide chain. For example, peptide linkers may be used to connect a cytokine moiety to a HSA domain.

Linkers suitable for fusing the extended-PK group to e.g. IL2 are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In addition to, or in place of, the heterologous polypeptides described above, a cytokine variant polypeptide described herein can contain sequences encoding a “marker” or “reporter”. Examples of marker or reporter genes include 8-lactamase, chloramphenicol acetyltransferase (CAT), adenosine deaminase (ADA), aminoglycoside phosphotransferase, dihydrofolate reductase (DHFR), hygromycin-B-hosphotransferase (HPH), thymidine kinase (TK), 8-galactosidase, and xanthine guanine phosphoribosyltransferase (XGPRT).

### Antigen

The subject-matter disclosed herein may involve immune effector cells that may be generated in a subject by means of active immunization, e.g., by vaccination using a peptide described herein, or passive immunization, e.g., by administering immune effector cells having a certain specificity wherein the immune effector cells may be genetically modified as described herein. The immune effector cells, in particular immune effector cells expressing an antigen receptor, e.g., immune effector cells which are genetically manipulated to express an antigen receptor, may be contacted in the subject being treated, with a cognate antigen molecule (also referred herein to as “antigen targeted by the antigen receptor”, “vaccine antigen” or simply “antigen”), wherein the antigen molecule or a procession product thereof, e.g., a fragment thereof, binds to the antigen receptor such as TCR carried by the immune effector cells, in particular when presented by MHC. In one embodiment, the cognate antigen molecule comprises the antigen expressed by a target cell to which the immune effector cells are targeted or a fragment thereof, or a variant of the antigen or the fragment.

Accordingly, the methods described herein comprise the step of administering the cognate antigen molecule, a nucleic acid coding therefor or cells expressing the cognate antigen molecule to the subject. In one embodiment, the nucleic acid encoding the cognate antigen molecule is expressed in cells of the subject to provide the cognate antigen molecule. In one embodiment, the nucleic acid encoding the cognate antigen molecule is transiently expressed in cells of the subject. In one embodiment, the nucleic encoding the cognate antigen molecule is RNA. In one embodiment, the cognate antigen molecule or the nucleic acid coding therefor is administered systemically. In one embodiment, after systemic administration of the nucleic acid encoding the cognate antigen molecule, expression of the nucleic acid encoding the cognate antigen molecule in spleen occurs. In one embodiment, after systemic administration of the nucleic acid encoding the cognate antigen molecule, expression of the nucleic acid encoding the cognate antigen molecule in antigen presenting cells, preferably professional antigen presenting cells occurs. In one embodiment, the antigen presenting cells are selected from the group consisting of dendritic cells, macrophages and B cells. In one embodiment, after systemic administration of the nucleic acid encoding the cognate antigen molecule, no or essentially no expression of the nucleic acid encoding the cognate antigen molecule in lung and/or liver occurs. In one embodiment, after systemic administration of the nucleic acid encoding the cognate antigen molecule, expression of the nucleic acid encoding the cognate antigen molecule in spleen is at least 5-fold the amount of expression in lung. In one embodiment, the cognate antigen molecule is processed and a procession product thereof is presented in the context of MHC for TCR recognition.

A peptide and protein antigen which is provided to a subject according to the invention (either by administering the peptide and protein antigen, a nucleic acid, in particular RNA, encoding the peptide and protein antigen or cells expressing the peptide and protein antigen), i.e., a vaccine antigen, preferably results in stimulation, priming and/or expansion of immune effector cells in the subject being administered the peptide or protein antigen, nucleic acid or cells. Said stimulated, primed and/or expanded immune effector cells are preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of immune effector cells which stimulated, primed and/or expanded immune effector cells target the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of stimulating, priming and/or expanding immune effector cells carrying an antigen receptor binding to the antigen or cells expressing the antigen. It is preferred that the vaccine antigen (similar to the disease-associated antigen) provides the relevant epitope for binding by the antigen receptor present in the immune effector cells. In one embodiment, the vaccine antigen (similar to the disease-associated antigen) is expressed by and presented on the surface of a cell such as an antigen-presenting cell in the context of MHC so as to provide the relevant epitope for binding by immune effector cells. The vaccine antigen may be a recombinant antigen.

In one embodiment of all aspects of the invention, the nucleic acid encoding the vaccine antigen is expressed in cells of a subject to provide the antigen or a procession product thereof for binding by the antigen receptor expressed by immune effector cells, said binding resulting in stimulation, priming and/or expansion of the immune effector cells.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject such as T cells binding to the reference amino acid sequence or cells expressing the reference amino acid sequence induces an immune reaction having a specificity of reacting with the reference amino acid sequence. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, may refer to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

The term “antigen” relates to an agent comprising an epitope against which an immune response can be generated. The term “antigen” includes, in particular, proteins and peptides. In one embodiment, an antigen is presented on the surface of cells of the immune system such as antigen presenting cells like dendritic cells or macrophages. An antigen or a procession product thereof such as a T cell epitope is in one embodiment bound by an antigen receptor. Accordingly, an antigen or a procession product thereof may react specifically with immune effector cells such as T-lymphocytes (T cells). In one embodiment, an antigen is a disease-associated antigen, such as a tumor antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “tumor antigen” or “tumor-associated antigen” refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, pI90 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

In preferred embodiments, an antigen is a tumor-associated antigen such as NY-ESO-1, MAGE-A3, tyrosinase and KRAS, respectively, and the present invention involves the stimulation of an anti-tumor CTL response against malignant cells expressing such tumor-associated antigen and preferably presenting such tumor-associated antigen with class I MHC.

NY-ESO-1 is a cancer/testis antigen expressed in normal adult tissues solely in the testicular germ cells of normal adults and in various cancers. It induces specific humoral and cellular immunity in patients with NY-ESO-1-expressing cancer.

The term “NY-ESO-1” preferably relates to human NY-ESO-1, and, in particular, to a protein comprising the amino acid sequence according to SEQ ID NO: 1 of the sequence listing or a variant of said amino acid sequence.

Whenever according to the various aspects of the invention NY-ESO-1, in particular SEQ ID NO: 1, an epitope sequence of NY-ESO-1, in particular SEQ ID NOs: 39, 40, 41, 42, and 101, respectively, or a T cell receptor sequence specific for NY-ESO-1, in particular SEQ ID NOs: 5 to 22, 91, and 92, is involved, the aim is preferably to induce an immune response against malignant cells expressing NY-ESO-1 and preferably being characterized by presentation of NY-ESO-1, and/or to treat or prevent a malignant disease involving cells expressing NY-ESO-1. Preferably the immune response involves the stimulation of an anti-NY-ESO-1 CTL response against malignant cells expressing NY-ESO-1 and preferably presenting NY-ESO-1 with class I MHC.

MAGE-A3 is melanoma-associated antigen 3 (MAGE-A3). MAGE-A3 is a tumor-specific protein, and has been identified on many tumors including melanoma, non-small cell lung cancer, hematologic malignancies, among others.

The term “MAGE-A3” preferably relates to human MAGE-A3, and, in particular, to a protein comprising the amino acid sequence according to SEQ ID NO: 2 of the sequence listing or a variant of said amino acid sequence.

Whenever according to the various aspects of the invention MAGE-A3, in particular SEQ ID NO: 2, an epitope sequence of MAGE-A3, in particular SEQ ID NOs: 43 and 44, respectively, or a T cell receptor sequence specific for MAGE-A3, in particular SEQ ID NOs: 23 to 34, is involved, the aim is preferably to induce an immune response against malignant cells expressing MAGE-A3 and preferably being characterized by presentation of MAGE-A3, and/or to treat or prevent a malignant disease involving cells expressing MAGE-A3. Preferably the immune response involves the stimulation of an anti-MAGE-A3 CTL response against malignant cells expressing MAGE-A3 and preferably presenting MAGE-A3 with class I MHC.

Tyrosinase is an oxidase that is the rate-limiting enzyme for controlling the production of melanin. Normally tyrosinase is produced in minute quantities but its levels are very much elevated in melanoma cells.

The term “tyrosinase” preferably relates to human tyrosinase, and, in particular, to a protein comprising the amino acid sequence according to SEQ ID NO: 3 of the sequence listing or a variant of said amino acid sequence.

Whenever according to the various aspects of the invention tyrosinase, in particular SEQ ID NO: 3, an epitope sequence of tyrosinase, or a T cell receptor sequence specific for tyrosinase, in particular SEQ ID NOs: 35 to 38, is involved, the aim is preferably to induce an immune response against malignant cells expressing tyrosinase and preferably being characterized by presentation of tyrosinase, and/or to treat or prevent a malignant disease involving cells expressing tyrosinase. Preferably the immune response involves the stimulation of an anti-tyrosinase CTL response against malignant cells expressing tyrosinase and preferably presenting tyrosinase with class I MHC.

The term “TPTE” relates to “transmembrane phosphatase with tensin homology”. The term “TPTE” preferably relates to human TPTE, and, in particular, to a protein comprising the amino acid sequence according to SEQ ID NO: 4 of the sequence listing or a variant of said amino acid sequence.

TPTE expression in healthy tissues is confined to testis and transcript amounts are below the detection limit in all other normal tissue specimens. In contrast, TPTE expression is found across different cancer types including malignant melanoma, breast cancer, lung cancer, prostate cancer, mammary cancer, ovarian cancer, renal cell carcinoma and cervical cancer.

TPTE transcription is initiated during the course of malignant transformation by cancer-associated DNA hypomethylation. Furthermore, TPTE promotes cancer progression and metastatic spread of cancer cells. In particular, TPTE is vital for efficient chemotaxis, a process which is involved in multiple aspects of cancer progression including cancer invasion and metastasis with impact on homing and metastatic destination of cancer cells. TPTE expression in primary tumors is associated with a significantly higher rate of metastatic disease.

Whenever according to the various aspects of the invention TPTE, in particular SEQ ID NO: 4, an epitope sequence of TPTE, or a T cell receptor sequence specific for TPTE is involved, the aim is preferably to induce an immune response against malignant cells expressing TPTE and preferably being characterized by presentation of TPTE, and/or to treat or prevent a malignant disease involving cells expressing TPTE. Preferably the immune response involves the stimulation of an anti-TPTE CTL response against malignant cells expressing TPTE and preferably presenting TPTE with class I MHC.

KRAS is a protein that is part of the RAS/MAPK pathway. The protein relays signals from outside the cell to the cell's nucleus instructing the cell to proliferate or differentiate. The KRAS protein is a GTPase and was first identified as an oncogene in Kirsten RAt Sarcoma virus. The KRAS gene is a proto-oncogene.

The term “KRAS” preferably relates to human KRAS, and, in particular, to a protein comprising the amino acid sequence according to SEQ ID NO: 90 of the sequence listing or a variant of said amino acid sequence, in particular a variant of said amino acid sequence wherein the glutamine at position 61 is replaced by histidine (Q61H).

Whenever according to the various aspects of the invention KRAS, in particular SEQ ID NO: 90 or SEQ ID NO: 90 (Q61H), an epitope sequence of KRAS, in particular SEQ ID NO: 102, or a T cell receptor sequence specific for KRAS, in particular SEQ ID NOs: 93 to 100, is involved, the aim is preferably to induce an immune response against malignant cells expressing KRAS and preferably being characterized by presentation of KRAS, and/or to treat or prevent a malignant disease involving cells expressing KRAS. Preferably the immune response involves the stimulation of an anti-KRAS CTL response against malignant cells expressing KRAS and preferably presenting KRAS with class I MHC.

The above described antigen sequences include any variants of said sequences, in particular mutants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. A species homolog is a nucleic acid or amino acid sequence with a different species of origin from that of a given nucleic acid or amino acid sequence. The terms “NY-ESO-1”, “MAGE-A3”, “tyrosinase”, “TPTE” and “KRAS” shall encompass (i) splice variants, (ii) posttranslationally modified variants, particularly including variants with different glycosylation such as N-glycosylation status, (iii) conformation variants, and (iv) disease related and non-disease related variants. Preferably, “NY-ESO-1”, “MAGE-A3”, “tyrosinase”, “TPTE” or “KRAS” is present in its native conformation.

“Target cell” includes a cell which is a target for an immune response such as a cellular immune response. Target cells include cells that present an antigen or an antigen epitope, i.e., a peptide fragment derived from an antigen, and include any undesirable cell such as a malignant cell as described herein. In preferred embodiments, the target cell is a cell expressing an antigen as described herein and preferably presenting said antigen with class I MHC.

The term “expressed on the cell surface” or “associated with the cell surface” means that a molecule such as a receptor is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells. In one embodiment, an antigen receptor expressed on the surface of cells is an integral membrane protein having an extracellular portion recognizing its antigen or a procession product thereof.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 8, 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

Preferably, the antigen peptides disclosed herein comprising an amino acid sequence selected from the group consisting of SEQ ID NOs: 39 to 44, 101, and 102 or a variant of said amino acid sequence are capable of stimulating an immune response, preferably a cellular response against the antigen from which they are derived or cells characterized by expression of the antigen and preferably characterized by presentation of the antigen. Preferably, an antigen peptide is capable of stimulating a cellular response against a cell characterized by presentation of the antigen with class I MHC and preferably is capable of stimulating an antigen-responsive CTL. Preferably, the antigen peptides according to the invention are MHC class I and/or class II presented peptides or can be processed to produce MHC class I and/or class II presented peptides. Preferably, the sequence bound to the MHC molecule is selected from SEQ ID NOs: 39 to 44, 101, and 102.

If an antigen peptide is to be presented directly, i.e. without processing, in particular without cleavage, it has a length which is suitable for binding to an MHC molecule, in particular a class I MHC molecule, and preferably is 7-20 amino acids in length, more preferably 7-12 amino acids in length, more preferably 8-11 amino acids in length, in particular 8, 9 or 10 amino acids in length. Preferably the sequence of an antigen peptide which is to be presented directly substantially corresponds and is preferably completely identical to a sequence selected from SEQ ID NOs: 39 to 44, 101, and 102.

If an antigen peptide is to be presented following processing, in particular following cleavage, the peptide produced by processing has a length which is suitable for binding to an MHC molecule, in particular a class I MHC molecule, and preferably is 7-20 amino acids in length, more preferably 7-12 amino acids in length, more preferably 8-11 amino acids in length, in particular 8, 9 or 10 amino acids in length. Preferably, the sequence of the peptide which is to be presented following processing substantially corresponds and is preferably completely identical to a sequence selected from SEQ ID NOs: 39 to 44, 101, and 102. Thus, an antigen peptide according to the invention in one embodiment comprises a sequence selected from SEQ ID NOs: 39 to 44, 101, and 102 and following processing of the antigen peptide makes up a sequence selected from SEQ ID NOs: 39 to 44, 101, and 102.

Peptides having amino acid sequences substantially corresponding to a sequence of a peptide which is presented by MHC molecules may differ at one or more residues that are not essential for TCR recognition of the peptide as presented by the MHC, or for peptide binding to MHC. Such substantially corresponding peptides preferably are also capable of stimulating an antigen-specific cellular response such as antigen-specific CTL. Peptides having amino acid sequences differing from a presented peptide at residues that do not affect TCR recognition but improve the stability of binding to MHC may improve the immunogenicity of the antigen peptide, and may be referred to herein as “optimized peptides”. Using existing knowledge about which of these residues may be more likely to affect binding either to the MHC or to the TCR, a rational approach to the design of substantially corresponding peptides may be employed. Resulting peptides that are functional are contemplated as antigen peptides. Sequences as discussed above are encompassed by the term “variant” used herein.

Cells such as antigen presenting cells can be loaded with MHC class I presented peptides by exposing, i.e. pulsing, the cells with the peptide or transducing the cells with nucleic acid, preferably RNA, encoding a peptide or protein comprising the peptide to be presented, e.g. a nucleic acid encoding the antigen.

In some embodiments, the invention involves an antigen presenting cell loaded with antigen peptide. In this respect, protocols may rely on in vitro culture/differentiation of dendritic cells manipulated in such a way that they artificially present antigen peptide. Production of genetically engineered dendritic cells may involve introduction of nucleic acids encoding antigens or antigen peptides into dendritic cells. Transfection of dendritic cells with mRNA is a promising antigen-loading technique of stimulating strong antitumor immunity. Such transfection may take place ex vivo, and a pharmaceutical composition comprising such transfected cells may then be used for therapeutic purposes. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75: 456-460,1997. Antigen loading of dendritic cells may be achieved by incubating dendritic cells or progenitor cells with antigen, DNA (naked or within a plasmid vector) or RNA; or with antigen-expressing recombinant bacteria or viruses (e.g., vaccinia, fowipox, adenovirus or lentivirus vectors).

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

According to the invention, the vaccine antigen should be recognizable by an immune effector cell. Preferably, the antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present invention, the antigen is preferably presented on the surface of a cell, preferably an antigen presenting cell. Recognition of the antigen on the surface of a diseased cell may result in an immune reaction against the antigen (or cell expressing the antigen).

### Chemotherapy

In certain embodiments, additional treatments may be administered to a patient in combination with the treatments described herein. Such additional treatments include classical cancer therapy, e.g., radiation therapy, surgery, hyperthermia therapy and/or chemotherapy.

Chemotherapy is a type of cancer treatment that uses one or more anti-cancer drugs (chemotherapeutic agents), usually as part of a standardized chemotherapy regimen. The term chemotherapy has come to connote non-specific usage of intracellular poisons to inhibit mitosis. The connotation excludes more selective agents that block extracellular signals (signal transduction). The development of therapies with specific molecular or genetic targets, which inhibit growth-promoting signals from classic endocrine hormones (primarily estrogens for breast cancer and androgens for prostate cancer) are now called hormonal therapies. By contrast, other inhibitions of growth-signals like those associated with receptor tyrosine kinases are referred to as targeted therapy.

Importantly, the use of drugs (whether chemotherapy, hormonal therapy or targeted therapy) constitutes systemic therapy for cancer in that they are introduced into the blood stream and are therefore in principle able to address cancer at any anatomic location in the body. Systemic therapy is often used in conjunction with other modalities that constitute local therapy (i.e. treatments whose efficacy is confined to the anatomic area where they are applied) for cancer such as radiation therapy, surgery or hyperthermia therapy.

Traditional chemotherapeutic agents are cytotoxic by means of interfering with cell division (mitosis) but cancer cells vary widely in their susceptibility to these agents. To a large extent, chemotherapy can be thought of as a way to damage or stress cells, which may then lead to cell death if apoptosis is initiated.

Chemotherapeutic agents include alkylating agents, antimetabolites, anti-microtubule agents, topoisomerase inhibitors, and cytotoxic antibiotics.

Alkylating agents have the ability to alkylate many molecules, including proteins, RNA and DNA. The subtypes of alkylating agents are the nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. They impair cell function by forming covalent bonds with the amino, carboxyl, sulfhydryl, and phosphate groups in biologically important molecules. Non-classical alkylating agents include procarbazine and hexamethylmelamine. In one particularly preferred embodiment, the alkylating agent is cyclophosphamide.

Anti-metabolites are a group of molecules that impede DNA and RNA synthesis. Many of them have a similar structure to the building blocks of DNA and RNA. Anti-metabolites resemble either nucleobases or nucleosides, but have altered chemical groups. These drugs exert their effect by either blocking the enzymes required for DNA synthesis or becoming incorporated into DNA or RNA. Subtypes of the anti-metabolites are the anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents block cell division by preventing microtubule function. The vinca alkaloids prevent the formation of the microtubules, whereas the taxanes prevent the microtubule disassembly. Vinca alkaloids include vinorelbine, vindesine, and vinflunine. Taxanes include docetaxel (Taxotere) and paclitaxel (Taxol).

Topoisomerase inhibitors are drugs that affect the activity of two enzymes: topoisomerase I and topoisomerase II and include irinotecan, topotecan, camptothecin, etoposide, doxorubicin, mitoxantrone, teniposide, novobiocin, merbarone, and aclarubicin.

The cytotoxic antibiotics are a varied group of drugs that have various mechanisms of action. The common theme that they share in their chemotherapy indication is that they interrupt cell division. The most important subgroup is the anthracyclines (e.g., doxorubicin, daunorubicin, epirubicin, idarubicin pirarubicin, and aclarubicin) and the bleomycins; other prominent examples include mitomycin C, mitoxantrone, and actinomycin.

In one embodiment, prior to administration of immune effector cells, a lymphodepleting treatment may be applied, e.g., by administering cyclophosphamide and fludarabine. Such treatment may increase cell persistence and the incidence and duration of clinical responses.

### Immune Checkpoint Inhibitors

In certain embodiments, immune checkpoint inhibitors are used in combination with other therapeutic agents described herein.

As used herein, “immune checkpoint” refers to co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1. In certain embodiments, the inhibitory signal is the interaction between CTLA-4 and CD80 or CD86 to displace CD28 binding. In certain embodiments the inhibitory signal is the interaction between LAG3 and MHC class II molecules. In certain embodiments, the inhibitory signal is the interaction between TIM3 and galectin 9.

As used herein, “immune checkpoint inhibitor” refers to a molecule that totally or partially reduces, inhibits, interferes with or modulates one or more checkpoint proteins. In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule that disrupts inhibitory signaling. In certain embodiments, the immune checkpoint inhibitor is an antibody, fragment thereof, or antibody mimic, that prevents the interaction between checkpoint blocker proteins, e.g., an antibody, or fragment thereof, that prevents the interaction between PD-1 and PD-L1. In certain embodiments, the immune checkpoint inhibitor is an antibody, or fragment thereof, that prevents the interaction between CTLA-4 and CD80 or CD86. In certain embodiments, the immune checkpoint inhibitor is an antibody, or fragment thereof, that prevents the interaction between LAG3 and its ligands, or TIM-3 and its ligands. The checkpoint inhibitor may also be in the form of the soluble form of the molecules (or variants thereof) themselves, e.g., a soluble PD-L1 or PD-L1 fusion.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1.

“Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1.

“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4.

“Lymphocyte Activation Gene-3 (LAG3)” is an inhibitory receptor associated with inhibition of lymphocyte activity by binding to MHC class II molecules. This receptor enhances the function of Treg cells and inhibits CD8⁺ effector T cell function. The term “LAG3” as used herein includes human LAG3 (hLAG3), variants, isoforms, and species homologs of hLAG3, and analogs having at least one common epitope.

“T Cell Membrane Protein-3 (TIM3)” is an inhibitory receptor involved in the inhibition of lymphocyte activity by inhibition of TH1 cell responses. Its ligand is galectin 9, which is upregulated in various types of cancers. The term “TIM3” as used herein includes human TIM3 (hTIM3), variants, isoforms, and species homologs of hTIM3, and analogs having at least one common epitope.

The “B7 family” refers to inhibitory ligands with undefined receptors. The B7 family encompasses B7-H3 and B7-H4, both upregulated on tumor cells and tumor infiltrating cells.

In certain embodiments, the immune checkpoint inhibitor suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardoll, D., Nature. 12: 252-264, 2012.

In certain embodiments, the immune checkpoint inhibitor is an antibody or an antigen-binding portion thereof, that disrupts or inhibits signaling from an inhibitory immunoregulator. In certain embodiments, the immune checkpoint inhibitor is a small molecule that disrupts or inhibits signaling from an inhibitory immunoregulator.

In certain embodiments, the inhibitory immunoregulator is a component of the PD-1/PD-L1 signaling pathway. Accordingly, certain embodiments of the disclosure provide for administering to a subject an antibody or an antigen-binding portion thereof that disrupts the interaction between the PD-1 receptor and its ligand, PD-L1. Antibodies which bind to PD-1 and disrupt the interaction between the PD-1 and its ligand, PD-L1, are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity.

In certain embodiments, the inhibitory immunoregulator is a component of the CTLA4 signaling pathway. Accordingly, certain embodiments of the disclosure provide for administering to a subject an antibody or an antigen-binding portion thereof that targets CTLA4 and disrupts its interaction with CD80 and CD86.

In certain embodiments, the inhibitory immunoregulator is a component of the LAG3 (lymphocyte activation gene 3) signaling pathway. Accordingly, certain embodiments of the disclosure provide for administering to a subject an antibody or an antigen-binding portion thereof that targets LAG3 and disrupts its interaction with MHC class II molecules.

In certain embodiments, the inhibitory immunoregulator is a component of the B7 family signaling pathway. In certain embodiments, the B7 family members are B7-H3 and B7-H4. Accordingly, certain embodiments of the disclosure provide for administering to a subject an antibody or an antigen-binding portion thereof that targets B7-H3 or H4. The B7 family does not have any defined receptors but these ligands are upregulated on tumor cells or tumor-infiltrating cells. Preclinical mouse models have shown that blockade of these ligands can enhance anti-tumor immunity.

In certain embodiments, the inhibitory immunoregulator is a component of the TIM3 (T cell membrane protein 3) signaling pathway. Accordingly, certain embodiments of the disclosure provide for administering to a subject an antibody or an antigen-binding portion thereof that targets TIM3 and disrupts its interaction with galectin 9.

It will be understood by one of ordinary skill in the art that other immune checkpoint targets can also be targeted by antagonists or antibodies, provided that the targeting results in the stimulation of an immune response such as an anti-tumor immune response as reflected in, e.g., an increase in T cell proliferation, enhanced T cell activation, and/or increased cytokine production (e.g., IFN-γ, IL2).

### RNA Targeting

It is particularly preferred according to the invention that the peptides, proteins or polypeptides described herein, in particular the vaccine antigens, are administered in the form of RNA encoding the peptides, proteins or polypeptides described herein. In one embodiment, different peptides, proteins or polypeptides described herein are encoded by different RNA molecules.

In one embodiment, the RNA is formulated in a delivery vehicle. In one embodiment, the delivery vehicle comprises particles. In one embodiment, the delivery vehicle comprises at least one lipid. In one embodiment, the at least one lipid comprises at least one cationic lipid. In one embodiment, the lipid forms a complex with and/or encapsulates the RNA. In one embodiment, the lipid is comprised in a vesicle encapsulating the RNA. In one embodiment, the RNA is formulated in liposomes.

According to the disclosure, after administration of the RNA described herein, at least a portion of the RNA is delivered to a target cell. In one embodiment, at least a portion of the RNA is delivered to the cytosol of the target cell. In one embodiment, the RNA is translated by the target cell to produce the encoded peptide or protein.

Some aspects of the disclosure involve the targeted delivery of the RNA disclosed herein (e.g., RNA encoding vaccine antigen).

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

In the context of the present disclosure, the term “RNA lipoplex particle” relates to a particle that contains lipid, in particular cationic lipid, and RNA. Such cationic lipids are described above. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

An additional lipid may be incorporated to adjust the overall positive to negative charge ratio and physical stability of the RNA lipoplex particles. Such additional lipids are described above. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

In one embodiment of the targeted delivery of a cytokine such as IL2, the target organ is liver and the target tissue is liver tissue. The delivery to such target tissue is preferred, in particular, if presence of the cytokine in this organ or tissue is desired and/or if it is desired to express large amounts of the cytokine and/or if systemic presence of the cytokine, in particular in significant amounts, is desired or required.

In one embodiment, RNA encoding a cytokine is administered in a formulation for targeting liver. Such formulations are described herein above.

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

### Pharmaceutical Compositions

The nucleic acids, nucleic acid particles, peptides, proteins, polypeptides, RNA, RNA particles, immune effector cells and further agents, e.g., immune checkpoint inhibitors, described herein may be administered in pharmaceutical compositions or medicaments for therapeutic or prophylactic treatments and may be administered in the form of any suitable pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing a disease involving an antigen described herein such as a cancer disease such as those described herein.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises nucleic acids, nucleic acid particles, peptides, proteins, polypeptides, RNA, RNA particles, immune effector cells and/or further agents as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IL15, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for systemic administration. In another preferred embodiment, the systemic administration is by intravenous administration. In one embodiment of all aspects of the invention, RNA encoding an antigen is administered systemically.

The term “co-administering” as used herein means a process whereby different compounds or compositions (e.g., immune effector cells [which may be “administered” by in vivo generation in a subject], and antigen, polynucleotide encoding antigen, or host cell genetically modified to express antigen) are administered to the same patient. The different compounds or compositions may be administered simultaneously, at essentially the same time, or sequentially. The antigen, polynucleotide encoding antigen, or host cell genetically modified to express antigen in one embodiment is administered following administration or generation of immune effector cells, e.g., at least one day, such as 1 to 10 days or 1 to 5 days following administration or generation of immune effector cells. The antigen, polynucleotide encoding antigen, or host cell genetically modified to express antigen may be administered several times over time in constant or different time intervals, e.g., following administration or generation of immune effector cells, e.g., in time intervals of between 10 and 40 days, wherein the first administration of antigen, polynucleotide encoding antigen, or host cell genetically modified to express antigen may be at least one day, such as 1 to 10 days or 1 to 5 days following administration or generation of immune effector cells.

### Treatments

The agents, compositions and methods described herein can be used to treat a subject with a disease, e.g. a disease characterized by the presence of diseased cells expressing an antigen as described herein. Particularly preferred diseases are cancer diseases. Accordingly, the agents, compositions and methods may be useful in the treatment of a cancer disease wherein cancer cells express a tumor antigen as described herein.

Immunotherapy may be performed using any of a variety of techniques, in which agents provided herein function to remove antigen-expressing cells from a patient. Such removal may take place as a result of enhancing or inducing an immune response in a patient specific for an antigen or a cell expressing an antigen.

Within certain embodiments, immunotherapy may be active immunotherapy, in which treatment relies on the in vivo stimulation of the endogenous host immune system to react against diseased cells with the administration of immune response-modifying agents (such as peptides and nucleic acids as provided herein).

Within other embodiments, immunotherapy may be passive immunotherapy, in which treatment involves the delivery of agents with established tumor-immune reactivity (such as effector cells) that can directly or indirectly mediate antitumor effects and does not necessarily depend on an intact host immune system. Examples of effector cells include T lymphocytes (such as CD8+ cytotoxic T lymphocytes and CD4+T-helper lymphocytes), and antigen-presenting cells (such as dendritic cells and macrophages). T cell receptors specific for the peptides recited herein may be cloned, expressed and transferred into other effector cells for adoptive immunotherapy.

As noted above, immunoreactive peptides as provided herein may be used to rapidly expand antigen-specific T cell cultures in order to generate a sufficient number of cells for immunotherapy. In particular, antigen-presenting cells, such as dendritic cells, macrophages, monocytes, fibroblasts and/or B cells, may be pulsed with immunoreactive peptides or transfected with one or more nucleic acids using standard techniques well known in the art. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL2 (see, for example, Cheever et al. (1997), Immunological Reviews 157, 177).

Alternatively, a nucleic acid expressing a peptide recited herein may be introduced into antigen-presenting cells taken from a patient and clonally propagated ex vivo for transplant back into the same patient.

Transfected cells may be reintroduced into the patient using any means known in the art, preferably in sterile form by intravenous, intracavitary, intraperitoneal or intratumor administration.

Methods disclosed herein may involve the administration of autologous T cells that have been activated in response to a peptide or peptide-expressing antigen presenting cell. Such T cells may be CD4+ and/or CD8+, and may be proliferated as described above. The T cells may be administered to the subject in an amount effective to inhibit the development of a disease.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against diseased cells expressing an antigen such as cancer cells expressing a tumor antigen, and to treat a disease such as a cancer disease involving cells expressing an antigen such as a tumor antigen.

An immune response against an antigen may be elicited which may be therapeutic or partially or fully protective. Pharmaceutical compositions described herein are applicable for inducing or enhancing an immune response. Pharmaceutical compositions described herein are thus useful in a prophylactic and/or therapeutic treatment of a disease involving an antigen.

As used herein, “immune response” refers to an integrated bodily response to an antigen or a cell expressing an antigen and refers to a cellular immune response and/or a humoral immune response.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells.

By “cell characterized by presentation of an antigen”, “cell presenting an antigen”, “antigen presented by a cell”, “antigen presented” or similar expressions is meant a cell such as a diseased cell such as a malignant cell, or an antigen presenting cell presenting the antigen it expresses or a fragment derived from said antigen, e.g. by processing of the antigen, in the context of MHC molecules, in particular MHC Class I molecules. Similarly, the terms “disease characterized by presentation of an antigen” denotes a disease involving cells characterized by presentation of an antigen, in particular with class I MHC. Presentation of an antigen by a cell may be effected by transfecting the cell with a nucleic acid such as RNA encoding the antigen.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naïve T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen”, “disease involving cells expressing an antigen” or similar terms refer to any disease which implicates an antigen, e.g. a disease which is characterized by the presence of an antigen. The disease involving an antigen can be a cancer disease or simply cancer. As mentioned above, the antigen may be a disease-associated antigen, such as a tumor-associated antigen. In one embodiment, a disease involving an antigen is a disease involving cells expressing an antigen, preferably a disease involving cells presenting an antigen.

Malignancy is the tendency of a medical condition, especially tumors, to become progressively worse and to potentially result in death. It is characterized by the properties of anaplasia, invasiveness, and metastasis. Malignant is a corresponding adjectival medical term used to describe a severe and progressively worsening disease. The term “malignant disease” as used herein preferably relates to cancer or a tumor disease. Similarly, the term “malignant cells” as used herein preferably relates to cancer cells or tumor cells. A malignant tumor may be contrasted with a non-cancerous benign tumor in that a malignancy is not self-limited in its growth, is capable of invading into adjacent tissues, and may be capable of spreading to distant tissues (metastasizing), while a benign tumor has none of those properties. Malignant tumor is essentially synonymous with cancer. Malignancy, malignant neoplasm, and malignant tumor are essentially synonymous with cancer.

According to the invention, the term “tumor” or “tumor disease” refers to a swelling or lesion formed by an abnormal growth of cells (called neoplastic cells or tumor cells). By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

A benign tumor is a tumor that lacks all three of the malignant properties of a cancer. Thus, by definition, a benign tumor does not grow in an unlimited, aggressive manner, does not invade surrounding tissues, and does not spread to non-adjacent tissues (metastasize). Common examples of benign tumors include moles and uterine fibroids.

The term “benign” implies a mild and nonprogressive disease, and indeed, many kinds of benign tumors are harmless to the health. However, some neoplasms which are defined as “benign tumors” because they lack the invasive properties of a cancer, may still produce negative health effects. Examples of this include tumors which produce a “mass effect” (compression of vital organs such as blood vessels), or “functional” tumors of endocrine tissues, which may overproduce certain hormones (examples include thyroid adenomas, adrenocortical adenomas, and pituitary adenomas).

Benign tumors typically are surrounded by an outer surface that inhibits their ability to behave in a malignant manner. In some cases, certain “benign” tumors may later give rise to malignant cancers, which result from additional genetic changes in a subpopulation of the tumor's neoplastic cells. A prominent example of this phenomenon is the tubular adenoma, a common type of colon polyp which is an important precursor to colon cancer. The cells in tubular adenomas, like most tumors which frequently progress to cancer, show certain abnormalities of cell maturation and appearance collectively known as dysplasia. These cellular abnormalities are not seen in benign tumors that rarely or never turn cancerous, but are seen in other pre-cancerous tissue abnormalities which do not form discrete masses, such as pre-cancerous lesions of the uterine cervix. Some authorities prefer to refer to dysplastic tumors as “pre-malignant”, and reserve the term “benign” for tumors which rarely or never give rise to cancer.

Neoplasm is an abnormal mass of tissue as a result of neoplasia. Neoplasia (new growth in Greek) is the abnormal proliferation of cells. The growth of the cells exceeds, and is uncoordinated with that of the normal tissues around it. The growth persists in the same excessive manner even after cessation of the stimuli. It usually causes a lump or tumor. Neoplasms may be benign, pre-malignant or malignant.

“Growth of a tumor” or “tumor growth” according to the invention relates to the tendency of a tumor to increase its size and/or to the tendency of tumor cells to proliferate.

Preferably, a “malignant disease” according to the invention is a cancer disease or tumor disease, and a malignant cell is a cancer cell or tumor cell. Preferably, a “malignant disease” is characterized by cells expressing a tumor-associated antigen such as NY-ESO-1, MAGE-A3, tyrosinase, TPTE, and KRAS, respectively.

Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor but some, like leukemia, do not.

Cancers are classified by the type of cell that resembles the tumor and, therefore, the tissue presumed to be the origin of the tumor. These are the histology and the location, respectively.

Examples of cancers include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases. In one embodiment, the cancer is melanoma, in particular malignant melanoma. In one embodiment, the cancer is NSCLC.

The term “melanoma”, or “malignant melanoma”, relates to a type of cancer that develops from the pigment-containing cells known as melanocytes. Melanomas typically occur in the skin, but may rarely occur in the mouth, intestines, or eye (uveal melanoma).

The term “NSCLC”, or “Non-small-cell lung carcinoma”, relates to a type of epithelial lung cancer other than small-cell lung carcinoma (SCLC). NSCLC accounts for about 85% of all lung cancers. The most common types of NSCLC are squamous-cell carcinoma, large-cell carcinoma, and adenocarcinoma, but several other types occur less frequently. A few of the less common types are pleomorphic, carcinoid tumor, salivary gland carcinoma, and unclassified carcinoma.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, according to the invention, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

The term “solid tumor” or “solid cancer” as used herein refers to the manifestation of a cancerous mass, as is well known in the art for example in Harrison's Principles of Internal Medicine, 14th edition. Preferably, the term refers to a cancer or carcinoma of body tissues other than blood, preferably other than blood, bone marrow, and lymphoid system. For example, but not by way of limitation, solid tumors include cancers of the prostate, lung cancer, colorectal tissue, bladder, oropharyngeal/laryngeal tissue, kidney, breast, endometrium, ovary, cervix, stomach, pancreas, brain, and central nervous system.

Combination strategies in cancer treatment may be desirable due to a resulting synergistic effect, which may be considerably stronger than the impact of a monotherapeutic approach. In one embodiment, the pharmaceutical composition is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (ILIβ), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin α5β1), Votumumab (tumor antigen CTAA 16.88), Zalutumumab (EGFR), and Zanolimumab (CD4).

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

The techniques and methods used herein are described herein or carried out in a manner known per se and as described, for example, in Sambrook et al., Molecular Cloning: A Laboratory Manual, 2 nd Edition (1989) Cold Spring Harbor Laboratory Press, Cold Spring Harbor, N.Y. All methods including the use of kits and reagents are carried out according to the manufacturers' information unless specifically indicated.

### Example 1: Materials and Methods

### Patient Material

PBMCs for immune-monitoring were isolated by Ficoll-Hypaque (Amersham Biosciences) density gradient centrifugation from peripheral blood or Leukapheresis samples. Immature DCs (S. Holtkamp et al., Blood. 108, 4009-17 (2006)) or fast DCs (M. Dauer et al., J. Immunol. 170, 4069-76 (2003)) were generated as described previously.

### Cell Lines

The K562, LCLC-103H, NCI-H-460 and the SK-Mel-28 cell lines were obtained from ATCC. The SK-Mel-29 was obtained from the Memorial Sloan Kettering Cancer Center, NY-York. The SK-Mel-37 cell line is described in Carey T E et al., Proc Natl Acad Sci, 1976. The Jurkat T cell line that expresses a luciferase reporter driven by an NFAT-response element is manufactured by Promega.

### In Vitro Stimulation (IVS) of PBMCs

CD4⁺ and CD8⁺ T cells were isolated from cryopreserved PBMCs using microbeads (Miltenyi Biotec). IVS cultures were either set using RNA or peptides. For IVS with RNA CD4- or CD8-depleted PBMCs were electroporated after overnight rest with RNA encoding vaccine antigens, eGFP, Influenza matrix protein 1 (M1) or tetanus p2/p16 sequences (positive control for CD4⁺ and CD8⁺ T cells respectively), left to rest for 3 h at 37° C. and irradiated at 15 Gy. Overnight rested CD4⁺/CD8⁺ T cells and electroporated and irradiated antigen-presenting cells were then combined at an effector to target ratio of 2:1. For peptide IVS, CD4⁺ T cells were expanded in the presence of fast DCs (effector to target ratio (E:T=10:1) pulsed with OLPs encoding either MAGE-A3, tyrosinase, TPTE or NY-ESO-1. For the expansion of CD8⁺ T cells, CD4-depleted PBMCs were co-cultured with purified CD8⁺ T cells (E:T=1:10) in the presence of IL-4 and GM-CSF (each 1000 U/mL) and the respective peptides. One day after starting the IVS, fresh culture medium containing 10 U/ml IL-2 (Proleukin S, Novartis), and 5 ng/mL IL-15 (Peprotech) was added. CD8 IVS cultures stimulated with peptides additionally obtained IL-4 and GM-CSF (each 1000 U/mL). For tumour cell lysis experiments also peptide-pulsed bulk PBMCs were used for IVS and harvested after 6-8 days of culture. For longer cultures, IL-2 was replenished 7 days after setting up the IVS cultures. After 11 days of stimulation, cells were analyzed via flow cytometry and used in ELISpot assays.

### IFNγ ELISpot

Multiscreen filter plates (Merck Millipore), pre-coated with antibodies specific for IFNγ (Mabtech) were washed with PBS and blocked with X-VIVO 15 (Lonza) containing 2% human serum albumin (CSL-Behring) for 1-5 hours. 0.5-3×10⁵ effector cells/well were stimulated for 16-20 h with either peptides (ex vivo setting) or autologous DCs electroporated with RNA or loaded with peptides (after IVS), or HLA class I or II transfected K562 cells peptides (TCR validation), For analysis of ex vivo T-cell responses, cryopreserved PBMCs were subjected to ELISpot after a resting period of 2-5 hours at 37° C. Alternatively, CD4- or CD8-depleted PBMCs were used as CD8 or CD4 effectors. All tests were performed in duplicate or triplicate and included positive controls (Staphyloccocus Enterotoxin B (Sigma Aldrich), anti-CD3 (Mabtech)) as well as cells from a reference donor with known reactivity. Spots were visualized with a biotin-conjugated anti-IFN [symbol] antibody (Mabtech) followed by incubation with ExtrAvidin-Alkaline Phosphatase (Sigma-Aldrich) and BCIP/NBT substrate (Sigma-Aldrich). Alternatively, a secondary antibody directly conjugated with ALP was used (ELISpotPro kit, Mabtech). Plates were scanned using CTL's ImmunoSpot® Series S five Versa ELISpot Analyzer (S5Versa-02-9038) or an AID Classic Robot ELISPOT Reader and analyzed by ImmunoCapture V6.3 or AID ELISPOT 7.0 software. Spot counts were summarized as median values for each triplicate or duplicate. T-cell responses stimulated by vaccine RNA or peptides were compared to control RNA (Luciferase) electroporated target cells or unloaded target cells, respectively. A response was defined as positive with a minimum of five spots per 1×10⁵ cells in the ex vivo setting or 25 spots per 5×10⁴ cells in the post-IVS setting as well as a spot count that was more than twice as high as the respective control.

### Flow Cytometry

Antigen-specific CD8⁺ T cells were identified using fluorophore coupled HLA multimers (Immudex). Cells were first stained with multimers followed by staining of cell surface markers (antibody clone in brackets) CD28 (CD28.8), CD197 (150503), CD45RA (HI100); CD3 (UCHT1 or SK7), CD16 (3G8), CD14 (M φP9), CD19 (SJ25C1), CD27 (L128), CD279 (EH12), CD134 (ACT35), CD8 (RPA-T8), all purchased from BD; CD19 (HIB19), CD4 (OKT4) from Biolegend as well as live-dead staining using DAPI (BD) or Fixable Viability Dye eFluor™ 780 (eBioscience). Singlet, live, multimer-positive events were identified within CD3 (or CD8) positive, CD4/CD14/CD16/CD19 negative or CD3 (or CD8) positive/CD4 negative events. For detection of antigen-specific T cells after IVS single, live, CD3⁺, CD8⁺/multimer⁺ lymphocytes were gated. In order to define the multimer positive population, the gate was set with respect to a fluorescence minus one (FMO) control.

For staining of intracellular cytokines, autologous DCs electroporated with RNA encoding single neo-epitopes were added at an E:T ratio of 10:1 and cultured for ˜16 h at 37° C. in the presence of Brefeldin A and Monensin. Cells were stained for viability dye (Fixable Viability Dye eFluor™ 506 or Viability Dye eFluor™ 780, eBioscience) and for surface markers CD8 (RPA-T8 or SK1), CD4 (SK3), CD16 (3G8), CD14 (M φP9), all purchased from BD; CD19 (HIB19), CD4 (OKT4) from Biolegend. After permeabilization, intracellular cytokine staining was performed using antibodies against IFNγ [symbol] (B27, BD) and TNF (Mab11, BD or Biolegend) for staining of intracellular cytokines. Singlet, live, IFNγ and TNF-positive events were identified within CD8 and CD4 positive, CD14/CD16/CD19 negative (if stained depending on marker panel used) events.

Cell surface expression of transfected TCR genes was analyzed using anti-TCR antibodies against the appropriate variable region family or the constant region of the TCR-13 chain (Beckman Coulter) and CD8 or CD4 specific antibodies (SK-1, BD). HLA antigens of the antigen presenting cells used for evaluating the function of TCR-transfected T cells were detected by staining with HLA class II-specific (9-49, Beckman Coulter and REA623, Miltenyi Biotec) and HLA class I-specific antibodies (DX17, BD Biosciences).

Acquisition was performed on a LSR Fortessa SORP, FACSCelesta or FACSCanto II (BD) and analyzed via FlowJo software (Tree Star).

### Cloning of HLA Antigens

HLA antigens were synthesized by Eurofins Genomics Germany GmbH according to respective high-resolution HLA typing results. HLA-DQA sequences were amplified from donor-specific cDNA with 2.5 U Pfu polymerase using DQA1_s (PHO-GCC ACC ATG ATC CTA MC AAA GCT CTG MTG C) and DQA1_as (TAT GCG ATC GCT CAC AAK GGC CCY TGG TGT CTG) primers. HLA antigens were cloned into appropriately digested IVT vectors (Simon, P. et al., Cancer Immunol. Res. 2, 1230-44 (2014)).

RNA Transfer into Cells

RNA was added to cells suspended in X-VIVO 15 medium (Lonza) in a precooled 4-mm gap sterile electroporation cuvette (Bio-Rad). Electroporation was performed with a BTX ECM 830 square wave electroporation system (T cells: 500 V/3 ms/1 pulse; iDC: 300 V/12 ms/1 pulse; bulk PBMCs: 400V/6 ms/1 pulse; MZ-GaBa-018: 225 V/3 ms/2 pulses; K562: 200 V/eight ms/3 pulses).

### Peptides

Overlapping peptide pools (PepMix™) encoding the whole length of NY-ESO-1, tyrosinase, MAGE-A3 and TPTE, or short (8-11-mer) epitopes derived from these antigens, the KRAS-Q61H55-64 epitope as well as control antigens (HIV-gag, SSX2) were used. All synthetic peptides were purchased from JPT Peptide Technologies GmbH and dissolved in water with 10% DMSO to a final concentration of 3 mM (short peptides) or in 100% DMSO (PepMix™).

### Single-Cell Sorting

Sorting of single antigen-specific T cells was conducted either using ex vivo PBMCs or IVS cultures based on stimulation-induced IFNγ secretion or multimer binding. For stimulation, PBMCs were pulsed with overlapping peptides covering the relevant antigen or a control antigen, while expanded T cells after IVS were cultured with autologous peptide-pulsed DCs. After 4 h, cells were harvested and treated with fluorochrome-conjugated antibodies directed against CD3, CD8, CD4, CD137 (4B4-1) (BD) as well as IFNγ using the IFNγ secretion assay kit (Miltenyi Biotec). Alternatively, PBMCs were stained with the respective multimer. Sorting of single neo-antigen-specific T cells was conducted on a FACSAria™ or a FACSMelody™ flow cytometer (both from BD Biosciences) using the BD FACSDiva™ and the BD FACSChorus™ software, respectively. Antigen-specific T cells were identified with respect to a control sample stimulated with a control antigen or stained without multimer. One T cell per well (gated on single, live CD3⁺ F and CD8⁺/IFNγ⁺, CD4⁺/IFNγ⁺ or CD8⁺/multimer⁺ lymphocytes or CD137⁺-IFNγ⁺ in CD3⁺ CD8⁺ gate) was harvested into 96-well V-bottom-plates (Greiner Bio-One) containing 6 μl of a mild hypotonic cell lysis buffer per well consisting of 0.2% Triton X-100, 0.2 μL RiboLock RNase inhibitor (Thermo Scientific), 5 ng Poly(A) carrier RNA (Qiagen) and 1 μL dNTP mix (10 mM, Biozym) in RNase free water. Plates were sealed, centrifuged and stored at −65° C. to −85 C directly after sorting.

### Cloning of Antigen-Specific TCRs

TCR genes were cloned from single T cells as previously described (M. Dauer, et al., J. Immunol. 170, 4069-76 (2003)) with the following modification. Plates with sorted cells were thawed and template-switch cDNA synthesis performed with RevertAid H-Reverse Transcriptase (Thermo Fisher) using TCR alpha and beta constant gene specific primers (TRAC 5′-catcacaggaactttctgggctg-3′, TRBC1 5′-gctggtaggacaccgaggtaaagc-3′ and TRBC2 5′-gctggtaagactcggaggtgaagc-3′) followed by preamplification using PfuUltra Hotstart DNA Polymerase (Agilent). Residual primers were removed after both cDNA synthesis and PCR by treatment with 5 U Exonuclease I (NEB). Aliquots of the cDNAs were used for Vα-/Vβ gene-specific multiplex PCRs. Products were analyzed on a capillary electrophoresis system (Qiagen). Samples with bands at 430 to 470 bp were size fractionated on agarose gels and the bands excised and purified using a Gel Extraction Kit (Qiagen). Purified fragments were sequenced and the respective V(D)J junctions analyzed using the IMGT/V-Quest tool³⁰. DNAs of novel and productively rearranged corresponding TCR chains were NotI-digested and cloned into pST1 vectors containing the appropriate constant region for in vitro transcription of complete TCR-α/β chains (Simon, P. et al., Cancer Immunol. Res. 2, 1230-44 (2014)).

Single Cell TCR (scTCR) Sequencing

For selected patients TCRs from sorted single cells were obtained by an NGS-based scTCRseq workflow. Here template-switch cDNA synthesis was performed using TCR alpha and beta constant gene specific primers (TRAC 5′-catcacaggaactttctgggctg-3′ and TRBC 5′-cacgtggtcggggwagaagc-3′) followed by a treatment with 5 U Exonuclease I. Each cDNA was PCR amplified and barcoded by row using 2.5 U PfuUltra Hotstart DNA Polymerase (Agilent), lx PCR buffer, 0.2 mM dNTPs, 0.2 μM of one of eight tagged forward primers Tag 130-RBCx-TS 5′-cgatccagactagacgctcaggaagxxx)o(aagcagtggtatcaacgcagagt-3′ and 0.1 μM of each tagged nested TCR alpha and beta constant gene specific primer Tag146-TRAC 5′-caatatgtgaccgccgagtcccaggttagagtctctcagctggtacacggcag-3′ and Tag 146-TRBC 5′-caatatgtgaccgccgagtcccaggggctcaaacacagcgacctcgggtg-3′ (95° C. for 2 min; 5 cycles of 94° C. for 30 s, 61° C. for 30 s, 72° C. for 1 min; 5 cycles of 94° C. for 30 s, 64° C. for 30 s, 72° C. for 1 min; 8 cycles of 94° C. for 30 s, 72° C. for 2 min; 72° C. 6 min). Samples of each column were pooled and purified twice using AMPure XP beads (Agencourt) with an Exonuclease I treatment in between. For each pool, a third of the purified TCR cDNA was further amplified by PCR using 1 NI PfuUltra II Fusion Hotstart DNA Polymerase (Agilent), lx reaction buffer, 0.2 mM dNTPs, forward primer Tag-130 5′-(n)nnnncgatccagactagacgctcaggaag-3′ and one of twelve Tag-146 reverse oligos containing a different barcode for each column 5′-xxxxxcaatatgtgaccgccgagtcccagg-3′ (95° C. for 1 min; 24 cycles of 94° C. for 20 s, 64° C. for 20 s, 72° C. for 30s; 72° C. 3 min). PCR products were pooled and purified with AMPure XP beads and Exonuclease I followed by generation of TCR sequencing libraries using the TruSeq DNA Nano kit (Illumina). The scTCR libraries were sequenced on an Illumina MiSeq with a sequencing depth of 10,000 reads per well using paired-end 300-base pair sequencing. Sequencing data were demultiplexed to a single-cell level using the bcl2fastq software (Illumina) followed by an in-house Python script. TCR sequences were then obtained using MiXCR-2.1.5 (Bolotin, D. A. et al., Nat. Methods 12, 380-1 (2015)). Selected paired alpha and beta VDJ fragments were synthesized (Eurofins Genomics) and cloned as described above for subsequent in vitro transcription.

### Bulk TCR Sequencing

Total RNA was isolated from 1×10⁶ snap frozen PBMCs collected at multiple time points during vaccination using the RNeasy Mini kit (QIAGEN). Libraries were produced with the SMARTer Human TCR a/b Profiling Kit (Clontech) and were sequenced using the Illumine MiSeq system. The number of total TCR reads per sample ranged from 1×10⁶ to 4×10⁶. Data were analyzed using VDJtools (Shugay, M. et al., PLoS Comput. Biol. 11, e1004503 (2015)) and MiXCR.

### Functional TCR Characterization

TCR-transfected CD4⁺ or CD8⁺ T cells from healthy donors were co-cultured with peptide-pulsed HLA-class I or II transfected K562 cells and tested by IFNγ-ELISpot assay. Alternatively, Jurkat cells of the T Cell Activation Bioassay (NFAT, Promega) were transfected with CD8α and TCRα/β encoding RNAs and tested against target cells. T cell activation was analyzed after addition of Bio-Glo Reagent (Promega) via luminescence measurement (Infinite F200 PRO, TECAN).

TCR-mediated cytotoxicity was assessed by cell index (CI) impedance measurements with the xCELLigence MP system (OMNI Life Science) according to the instructions of the supplier. Target cells were seeded at concentrations of 2×10⁴ cells per well in 96 well PET E-plates (ACEA Biosciences Inc.). After 24 h TCR-transfected T cells were added using different E:T ratios and monitored every 30 min for a period of up to 48 h by the xCELLigence system. Specific lysis was calculated after indicated times of coculture.

### Example 2: Discovery and Characterization of HLA Class I Restricted NY-ESO-1 Specific TCRs of LipoMERIT Patient A2-09 and 53-02

A systemically administered, nano-particulate liposomal RNA vaccine (RNA-LPX) was tested in a phase I dose-escalation trial in advanced melanoma patients (Lipo-MERIT, NCT02410733).

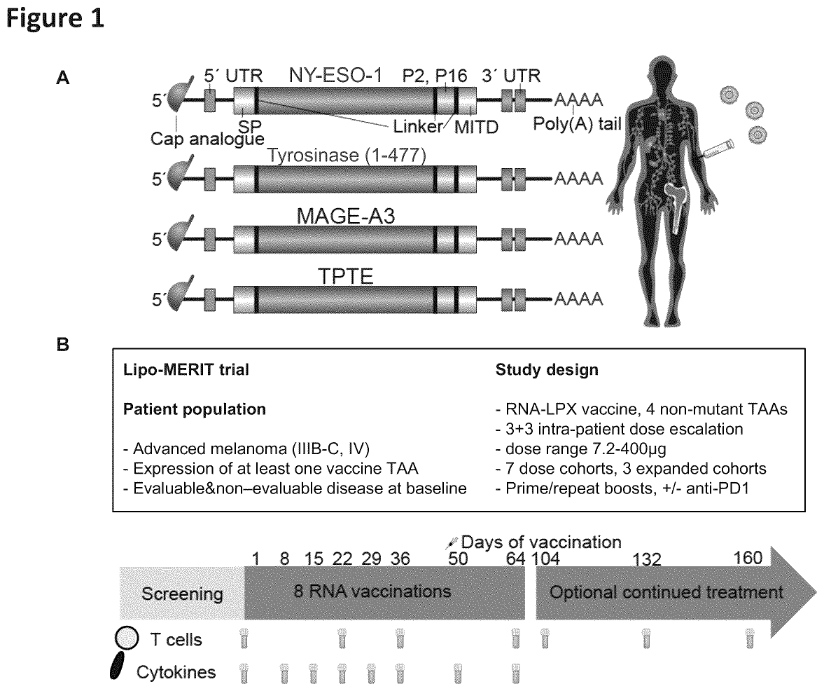

The vaccine (referred to as melanoma FixVac) is composed of four lipid-complexed RNAs encoding the non-mutant TAAs NY-ESO-1, MAGE-A3, tyrosinase and TPTE, each of which is known for its restricted expression in normal tissues, high immunogenicity and high prevalence in human melanoma (Simon, P. et al., Cancer Immunol. Res. 2, 1230-44 (2014); Cheever, M. A. et al., Clin. Cancer Res. 15, 5323-37 (2009)). The single-stranded, 5′-capped vaccine messenger RNA (FIG. 1A) bears functional sequence elements that improve efficiency of its translation specifically in immature DCs (Holtkamp, S. et al., Blood 108, 4009-17 (2006); Orlandini von Niessen, A. G. et al., Mol. Ther. (2018). doi:10.1016/j.ymthe.2018.12.011). The open reading frame of each TAA is fused in frame to an N-terminal secretion signal and C-terminally to tetanus toxoid helper epitopes and an endosomal targeting domain, which augments processing and presentation of tumour antigen-derived epitopes on the patient's individual HLA-class I and II molecules (Kreiter, S. et al., J. Immunol. 180, 309-318 (2008)).

Patients who express at least one of the TAAs confirmed per qRT-PCR analysis were eligible for this trial. Melanoma FixVac was administered according to a prime/repeat boosts protocol followed by optional continued monthly treatment (FIG. 1B).

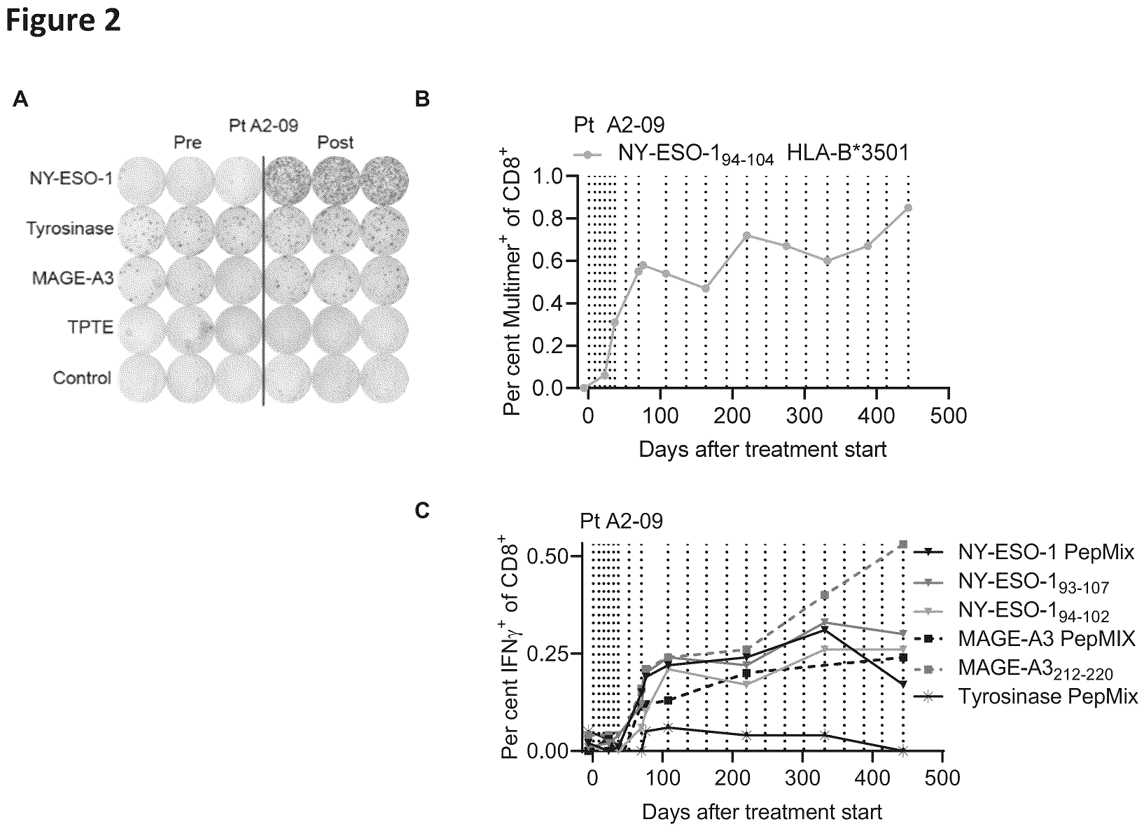

To determine the immunogenicity of the four RNA encoded TAAs, T-cell responses in pre- and post-vaccination blood samples of patient A2-009 were analysed by IFNγ-ELISPOT, multimer staining and ICS (FIG. 2A-C).

All three orthogonal assays demonstrated an induction and expansion of NY-ESO-1-specific CD8⁺ T cells after FixVac treatment. NY-ESO-1-specific T cells were barely detectable at baseline and showed a fast ramp up within the first 4-8 weeks nearly to 1 percent of circulating CD8⁺ T cells (FIG. 2B).

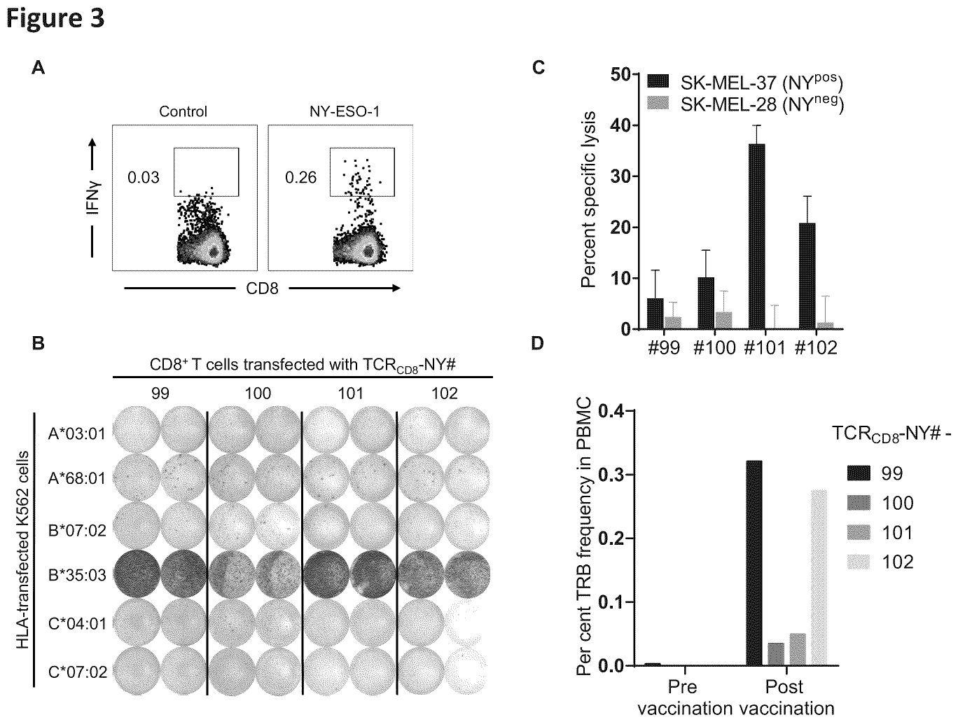

In order to analyse the vaccine induced NY-ESO-1-specific T cell response on a molecular level, TCR-α/β chains were cloned from single NY-ESO-1-specific T cells. To that aim, post treatment PBMCs were stimulated with NY-ESO-1 encoding overlapping 15mer peptides (OLPs) and single NY-ESO-1-specific T cells were sorted by flow cytometry based on activation-induced secretion of IFNγ (FIG. 3A). In vitro transcribed (IVT) RNAs of cloned TCR-α/β chains were co-transfected into T cells of a healthy donor for functional characterization. Specificity and HLA class I restriction was evaluated by IFNγ-ELISPOT using TCR-transfected CD8⁺ T cells co-cultured with K562 cells expressing individual HLA class I alleles of the patient and pulsed with NY-ESO-1 peptide pool. Notably, four NY-ESO-1-specific TCRs were identified and shown to be restricted to B*3503 (FIG. 3B, Table 1). In order to evaluate, if NY-ESO-1-specific TCRs isolated from CD8⁺ T cells are able to mediate cytolytic effector functions, the specific killing of NY-ESO-1-positive and -negative melanoma cell lines by pre-activated TCR-transfected CD8⁺ T cells was analyzed by impedance based cytotoxicity assay (FIG. 3C). Indeed, all TCRs mediated antigen-specific lysis of NY-ESO-1-positive SK-MEL-37 cells, while NY-ESO-1-negative SK-MEL-28 cells were not lysed. Tracking of NY-ESO-1-TCR-13 sequences in TCR deep-sequencing data revealed that frequencies in pre-vaccination PBMCs were not detectable or extremely low, while high clonotype frequencies were detected in post-vaccination PBMCs (FIG. 3D).

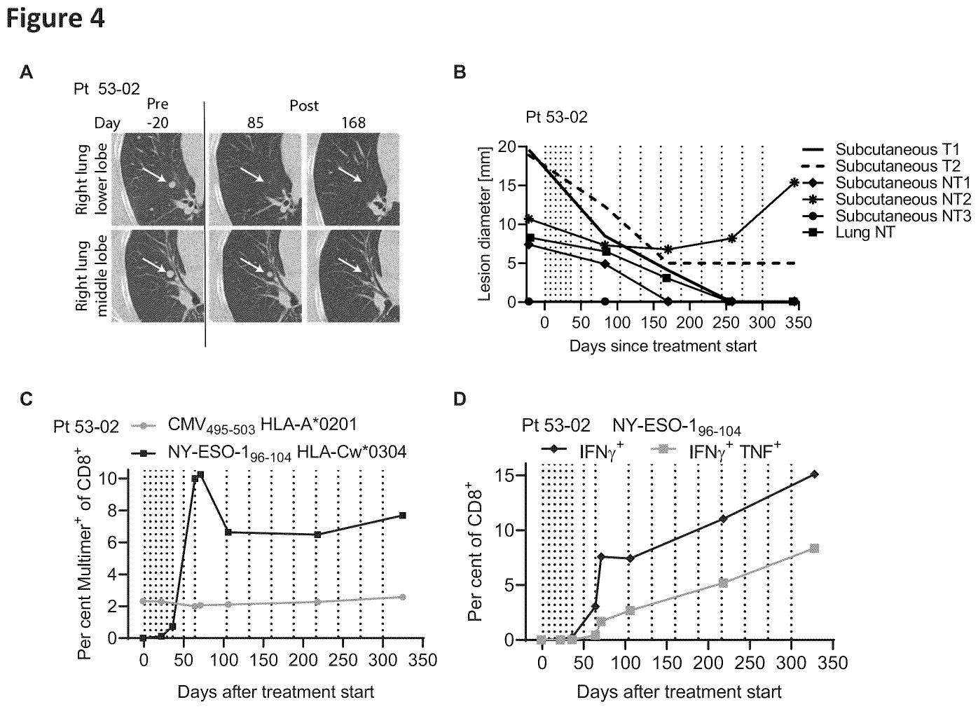

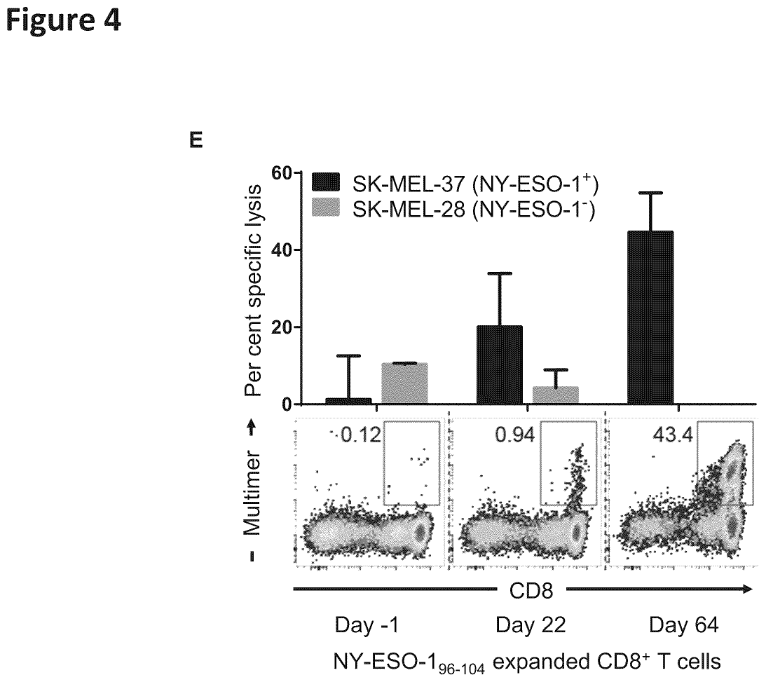

To study the contribution of melanoma FixVac to the treatment effects, immune responses of selected responder patients for whom blood samples were available were analyzed. Patient 53-02, who entered the trial after progressing under pembrolizumab treatment, experienced a PR of about 8 months duration on melanoma FixVac with regression of multiple lung and subcutaneous metastases (FIG. 4A, B). The patient mounted strong de novo immune responses against NY-ESO-1 and MAGE-A3 detectable by ex vivo ELISpot (data not shown). A vaccine-induced HLA-Cw*0304 restricted CD8⁺ T-cell response against the NY-ESO-1₉₆₋₁₀₄ epitope (Jackson, H. et al., J. Immunol. 176, 5908-17 (2006)) was identified by HLA multimer staining, which increased steeply to over 10% of peripheral blood CD8⁺ T cells and remained high under continued vaccination (FIG. 4C). Analysis of peripheral blood T cells by ICS as an orthogonal assay showed expansion of NY-ESO-1 reactive IFNγ⁺ T cells comprising up to 15% of the whole peripheral blood CD8⁺ T cell population (FIG. 4D). The vaccine-induced T cells exerted a strong antigen-specific cytotoxic activity as demonstrated by killing of endogenously NY-ESO-1 expressing melanoma cells by short-term IVS cultures from post-vaccination PBMCs expanded against the NY-ESO-1₉₆₋₁₀₄ epitope (FIG. 4E).

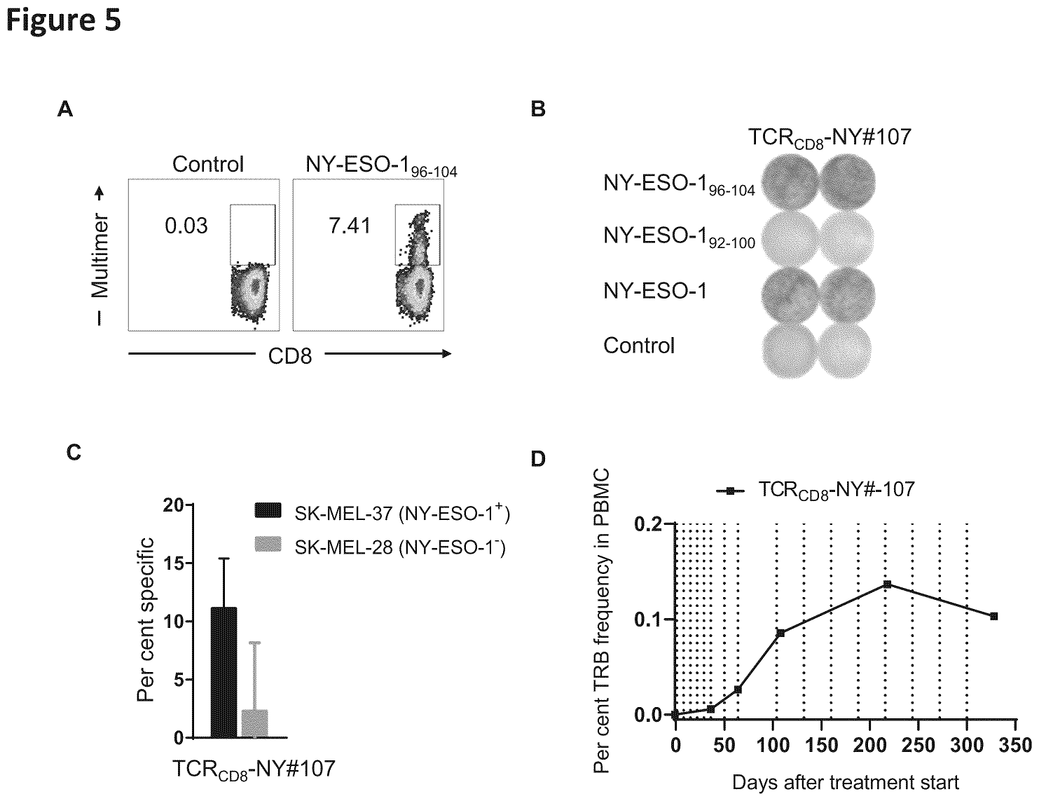

For further characterization of this population, we isolated single NY-ESO-1 specific T cells from post-vaccination blood samples based on their binding to HLA multimers (FIG. 5A). By single-cell TCR cloning we identified a HLA-Cw*0304-restricted TCR, TCR_{CD8}-53-02-NY #107 (FIG. 5B, Table 1), that was able to mediate lysis of NY-ESO-1 expressing SK-MEL-37 melanoma cells (FIG. 5C). TCR beta clonotype analyses of pre- and post-vaccination blood samples showed that the Cw*0304-restricted NY-ESO-1-TCR was not detectable at baseline and expanded through repeated vaccination (FIG. 5D).

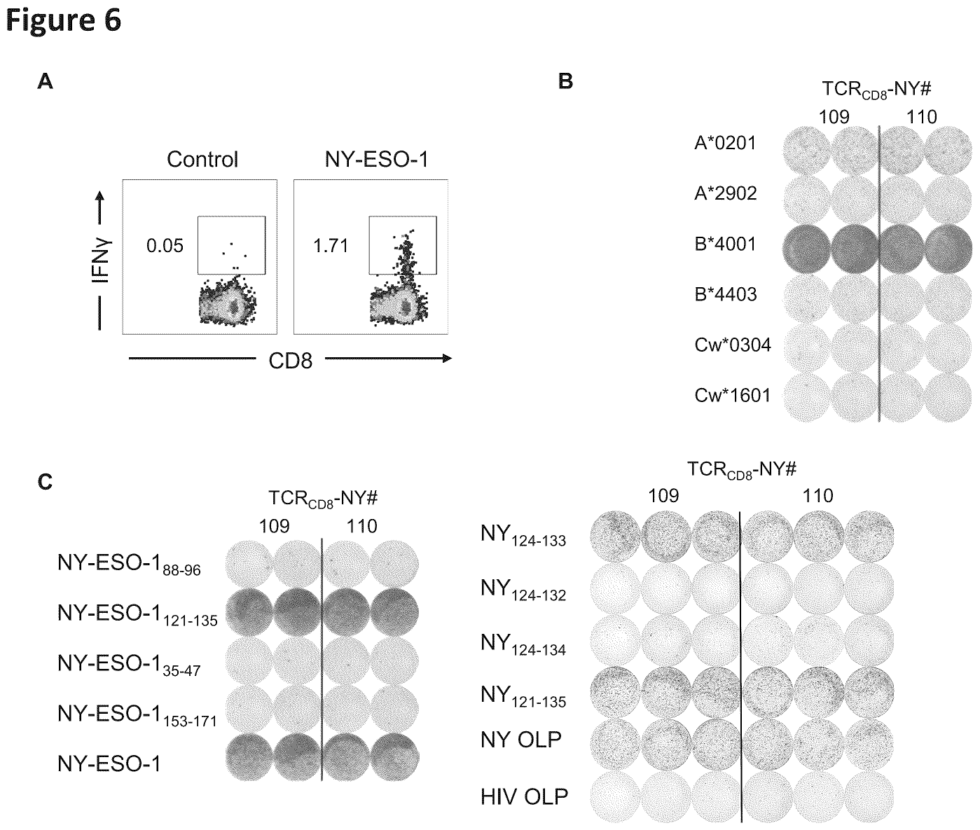

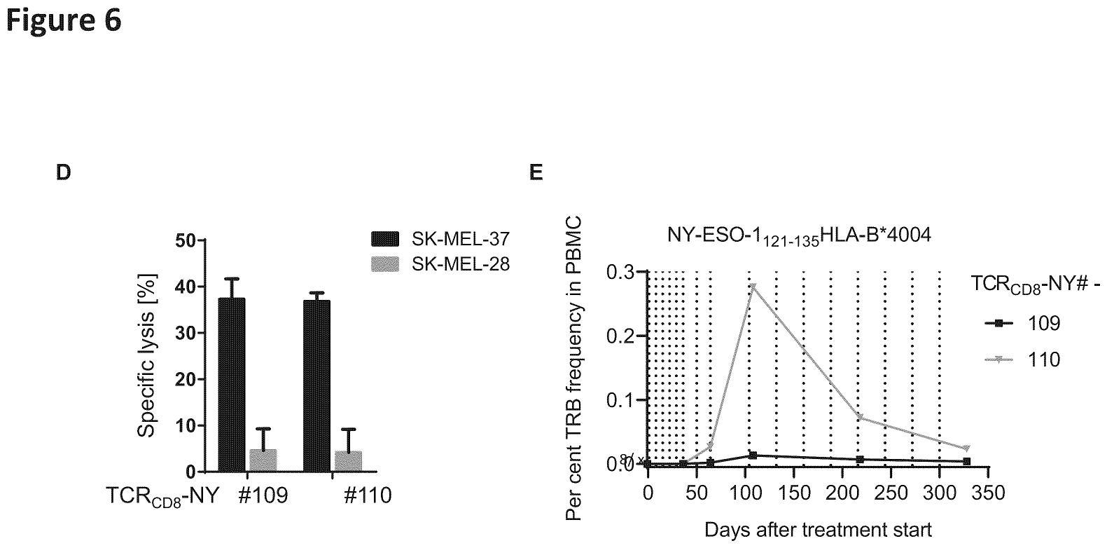

In order to identify also NY-ESO-1-TCRs recognizing other HLA presented NY-ESO-1 epitopes, post treatment PBMCs were stimulated with NY-ESO-1 encoding OLPs and CD8⁺ T cells were sorted based on antigen-specific IFNγ release (FIG. 6A). Two NY-ESO-1 TCRs, TCR_{CD8}-53-02-NY #109 and #110, were identified shown to recognize a B*4001-restricted NY-ESO-1 epitope (FIG. 6B, Table 1). Testing of different NY-ESO-1 peptides revealed that both TCRs recognize NY-ESO-1₁₂₄₋₁₃₃, an epitope that has not been described in the combination with HLA-B*4001, yet (FIG. 6C). Both NY-ESO-1-TCRs mediated efficient killing of endogenously NY-ESO-1 expressing melanoma cells SK-MEL-37 confirming that the identified epitope is endogenously processed and presented by melanoma cells (FIG. 6D). TCR beta clonotype analyses of pre- and post-vaccination blood samples showed that the B*4001-restricted NY-ESO-1-TCRs were not detectable at baseline and expanded through repeated vaccination (FIG. 6E).

These data indicate that the objective tumour response in this patient is associated with a vaccine-induced, poly-epitopic T-cell response against NY-ESO-1.

### Example 3: Discovery and Characterization of HLA Class I Restricted MAGE-A3 Specific TCRs of LipoMERIT Patient C2-28 and C1-40

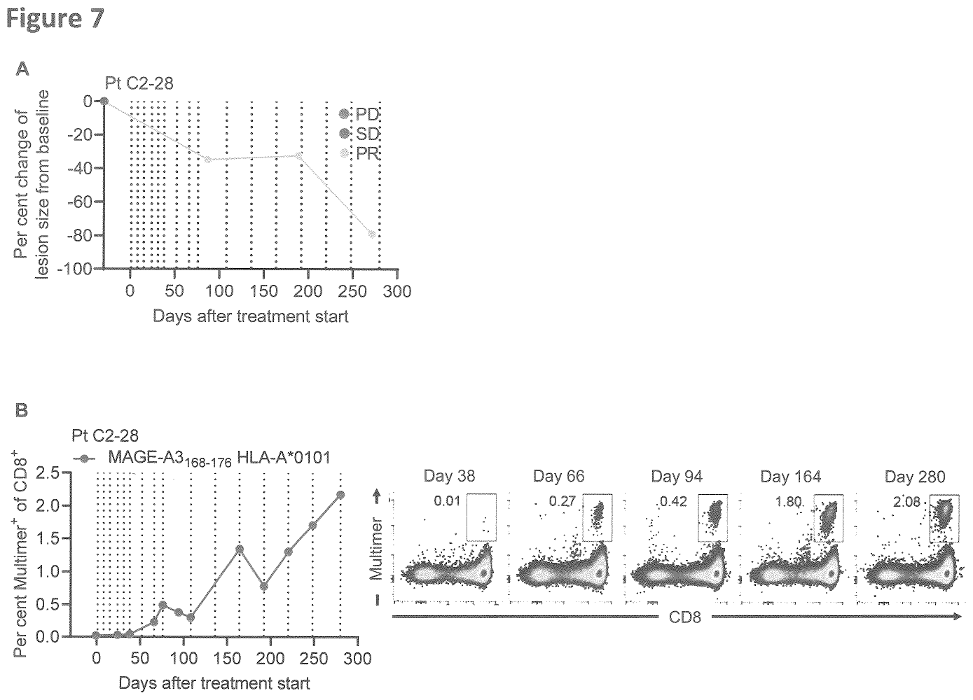

Patient C2-28 had a melanoma with multiple liver and subcutaneous metastases and entered the trial with radiologically confirmed progression under ipilimumab/nivolumab combination treatment. The patient was switched to combined treatment with melanoma FixVac and nivolumab and experienced a PR (FIG. 7A). After 8 vaccinations immune responses against NY-ESO-1 and MAGE-A3 were detected by IVS ELISpot (data not shown). HLA multimer staining revealed a vaccine-induced T-cell response against the previously described HLA-A*0101 restricted MAGE-A3₁₆₈₋₁₇₆ epitope (Hanagiri, T., et al., Cancer Immunol. Immunother. 55, 178-84 (2006)) detectable ex vivo two months after start of melanoma FixVac and sequentially increasing over the time course of several months to up to 2% of peripheral blood CD8⁺ T cells (FIG. 7B).

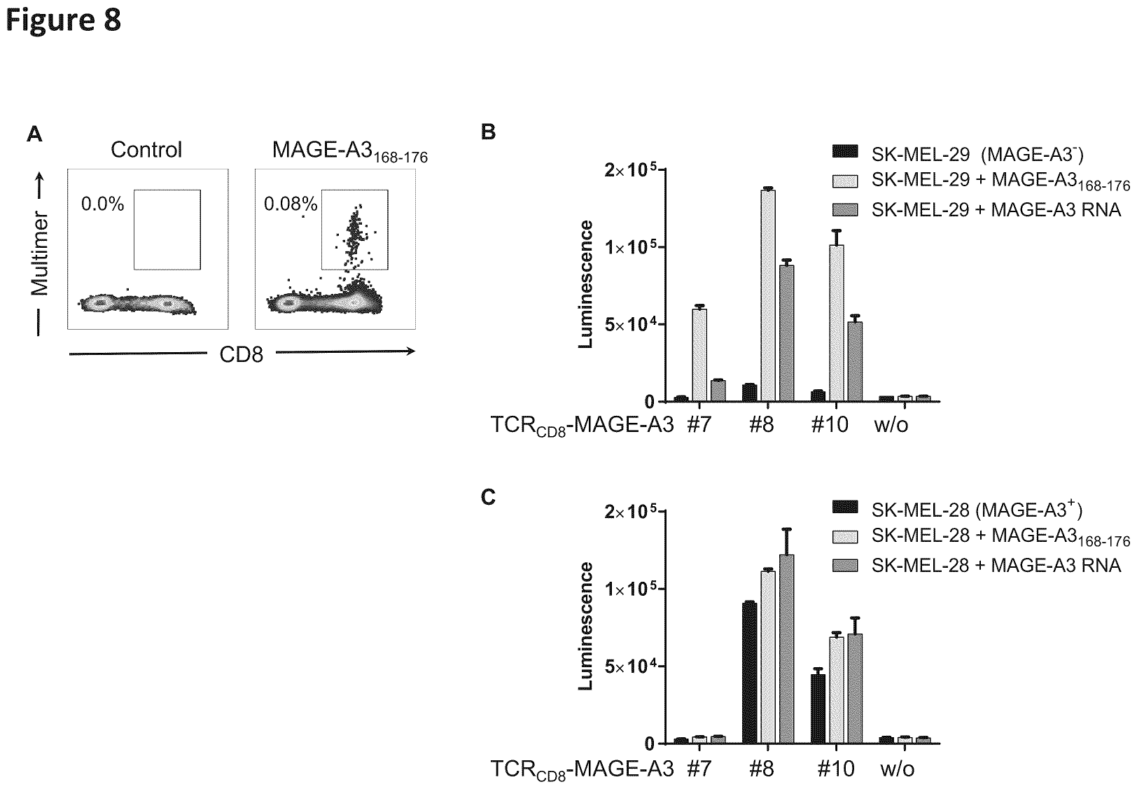

Three MAGE-A3-specific TCRs were discovered from post-vaccination MAGE-A3₁₆₈₋₁₇₆ multimer-specific T cells (FIG. 8A, Table 1). All three TCRs recognized MAGE-A3-negative target cells after pulsing with MAGE-A3 encoding RNA or MAGE-A3₁₆₈₋₁₇₆ peptide (FIG. 8B), while two of them showed specific recognition of the endogenously expressed MAGE-A3 on melanoma cells (FIG. 8C).

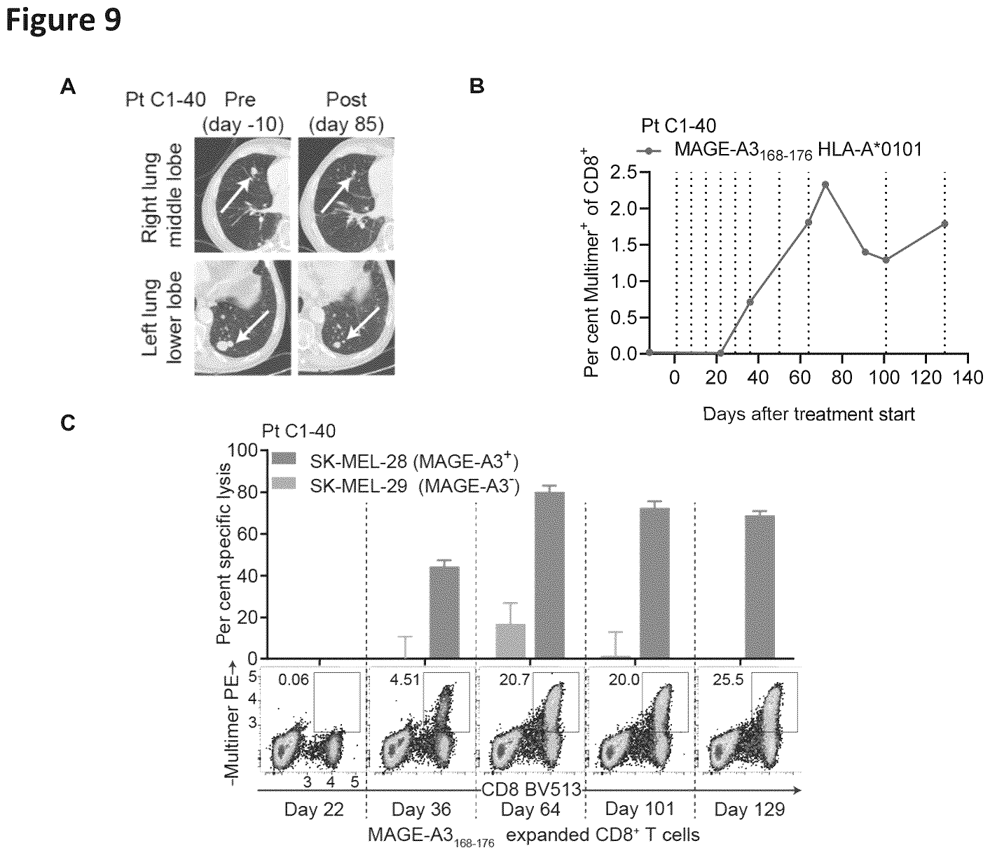

Patient C1-40 had a history of pembrolizumab-responsive metastatic melanoma and 7 months after discontinuation of anti-PD1 therapy experienced disease progression with multiple fast progressing lung lesions. Treatment with nivolumab was initiated, and 8 weeks thereafter melanoma FixVac was added on top of this anti-PD1 therapy. The patient experienced a partial response with concomitant shrinkage of the lung metastases (FIG. 9A). HLA multimer staining revealed a strong vaccine-induced T-cell response against the HLA-A*0101 restricted MAGE-A3₁₆₈₋₁₇₆ epitope (FIG. 9B). Notably, short-time cultures of lymphocytes obtained from post-vaccination blood samples exhibited a highly efficient killing of MAGE-A3 positive melanoma cells indicating functionality of these vaccine-induced MAGE-A3-specific T cells (FIG. 9C).

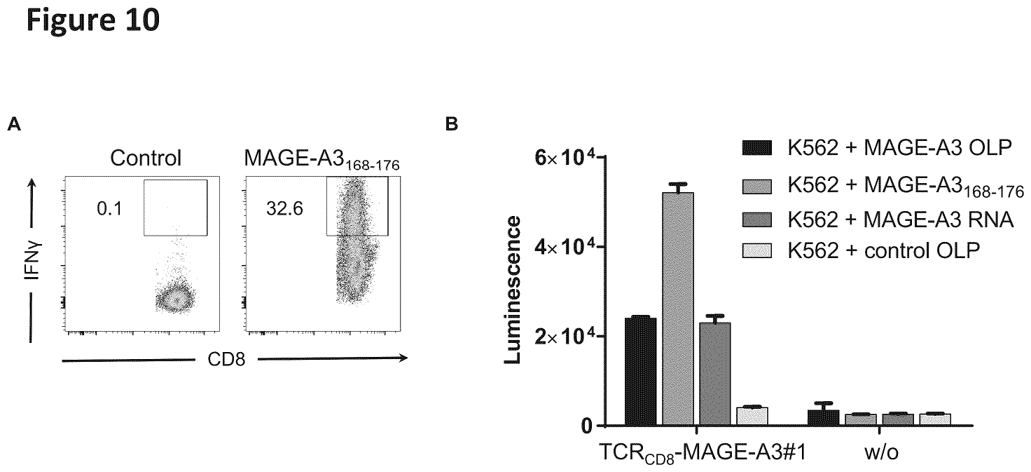

IVS cultures from post treatment PBMCs were used to discover a MAGE-A3₁₆₈₋₁₇₆-specific HLA A*0101-restricted TCR from CD8⁺ T cells of patient C1-40 secreting IFNγ after restimulation with MAGE-A3₁₆₈₋₁₇₆ peptide-pulsed iDCs (FIG. 10A, Table 1). TCR_{CD8}-C1-40-MA3 #1 was shown to mediate recognition of HLA-A*0101-transfected target cells either pulsed with MAGE-A3 peptides or transfected with MAGE-A3 RNA (FIG. 10B).

### Example 4: Discovery and Characterization of HLA Class II Restricted MAGE-A3, NY-ESO-1 and Tyrosinase Specific TCRs of LipoMERIT Patient A2-010

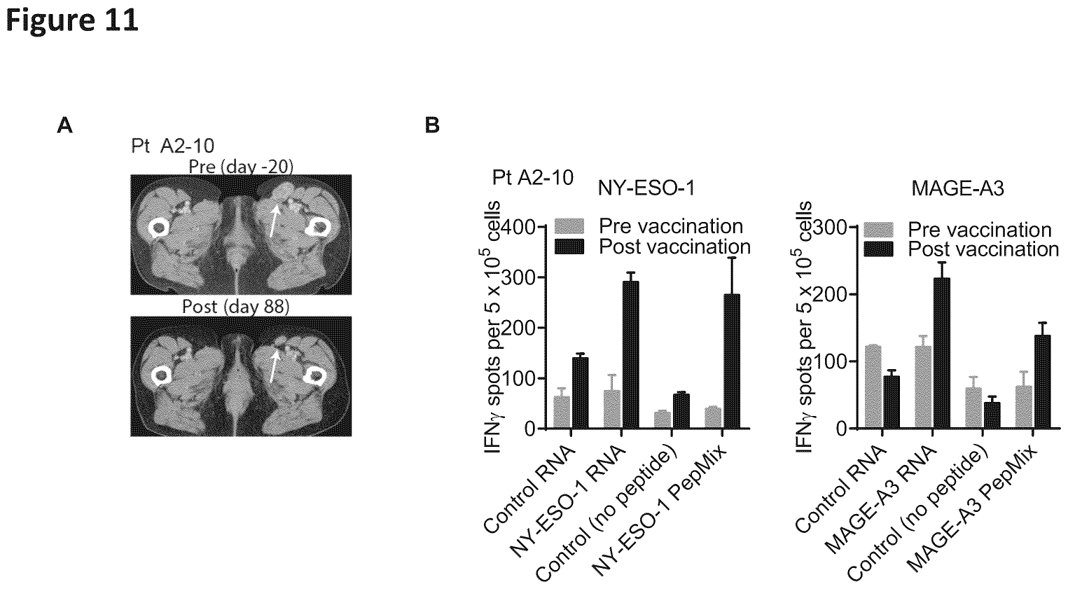

Patient A2-10 had a history of checkpoint inhibitor refractory melanoma with fast progressing multi-metastatic disease under ipilimumab and nivolumab. With melanoma FixVac monotherapy the patient experienced a partial response of 6-month duration with regression of multiple lymph node and lung metastases (FIG. 11A). We detected an IFNγ⁺/CD4⁺ T-cell response against MAGE-A3 and NY-ESO-1 in post-treatment PBMCs (FIG. 11B).

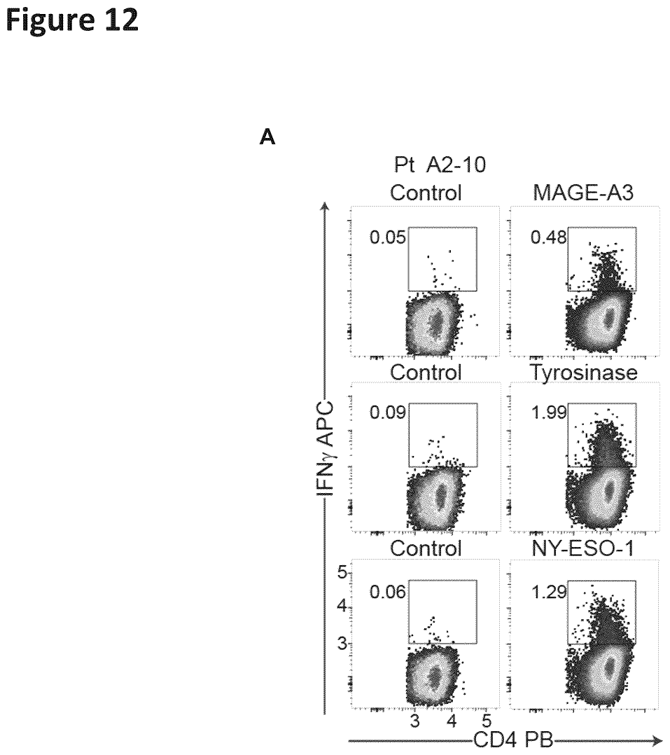

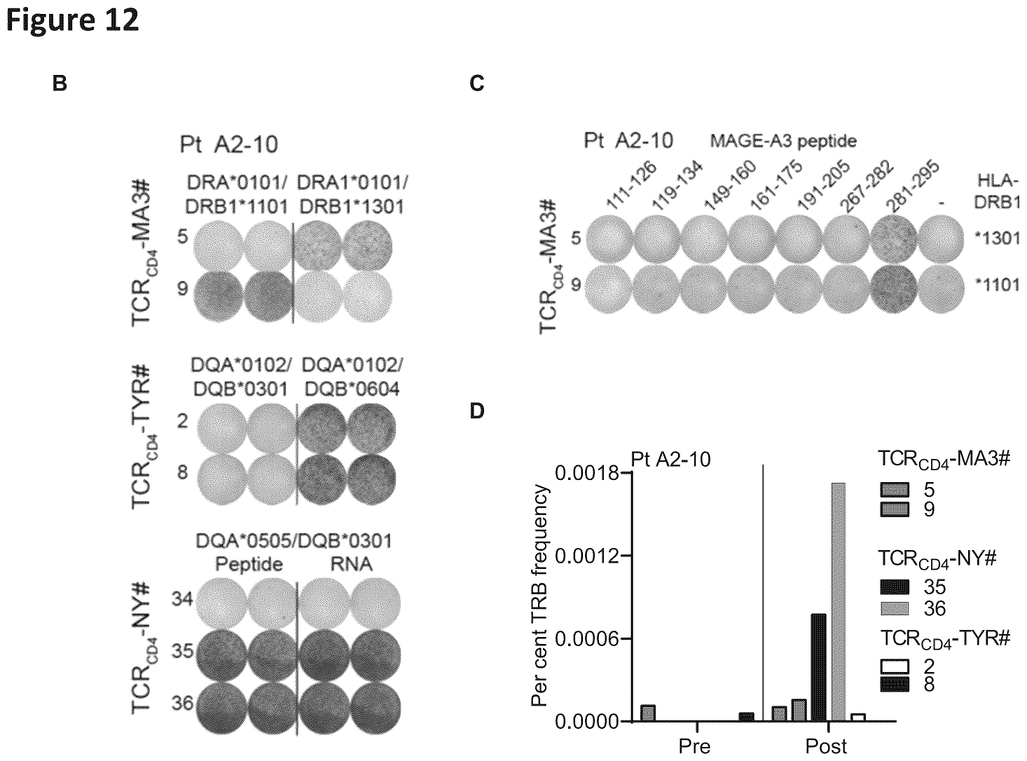

CD4⁺ T cells from IVS cultures were used to discover HLA class II restricted TAA-specific T cells after restimulaton with autologous iDCs pulsed with TAA encoding OLPs (FIG. 12A). Several NY-ESO-1-, tyrosinase-, and MAGE-A3-directed TCR clonotypes with different HLA class II restrictions were retrieved by single-cell cloning from CD4⁺ T cells (FIG. 12B, Table 1). Two MAGE-A3-TCRs recognized the MAGE-A3₂₈₁₋₂₉₅ epitope, previously reported as immune dominant and promiscuitively presented on various HLA-DRB1 alleles (Hu, Y. et al., Cancer Immunol. Immunother. 63, 779-86 (2014)) (FIG. 12C). Tracking of the identified clonotypes in TCR profiling data revealed that most of them were vaccine amplified with pre-vaccination frequencies below the detection threshold (FIG. 12D).

### Example 5: Discovery and Characterization of an HLA-A*0201-Restricted NY-ESO-1-Specific TCR from a Checkpoint Inhibitor Treated NSCLC Patient

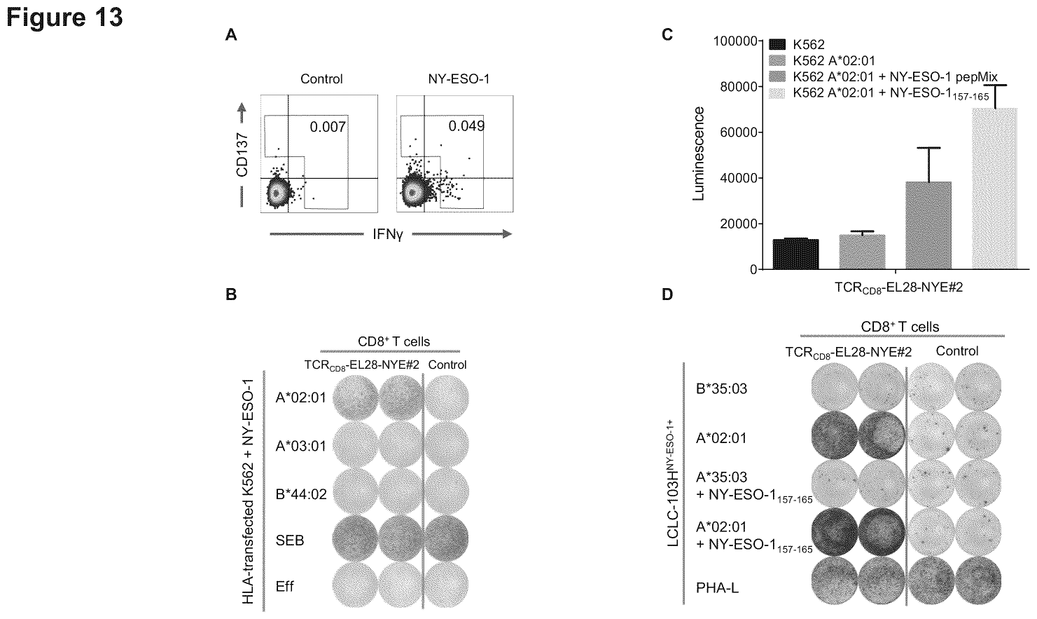

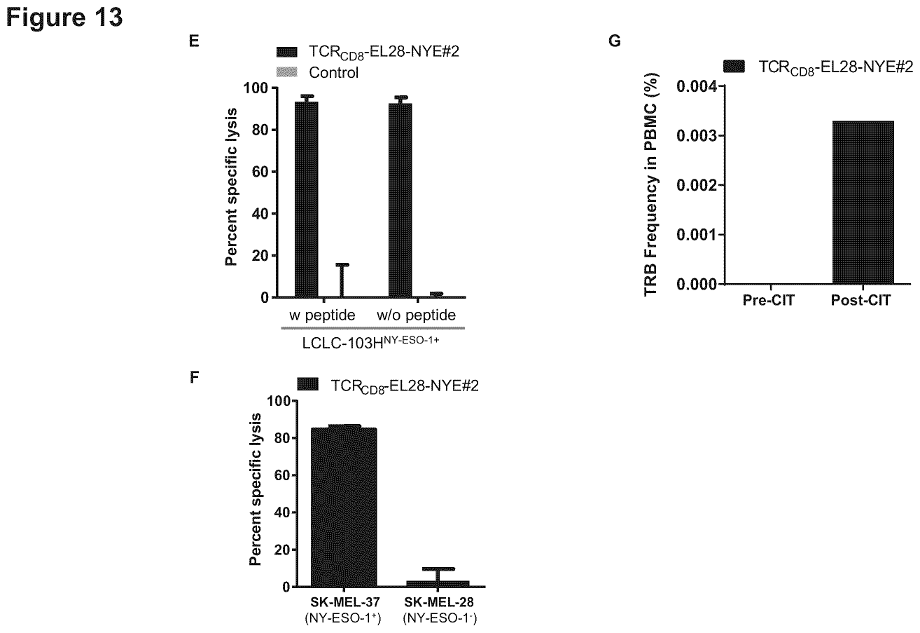

In order to discover functional TCRs that specifically recognize antigens expressed by tumor cells in non-small cell lung cancer (NSCLC), a collection of patient-derived biological materials was established including tumor tissues, tumor-infiltrating lymphocytes and PBMCs from NSCLC patients with or without prior checkpoint inhibitor therapy (CIT). Tumor tissues and autologous PBMCs were analyzed by RNA sequencing to identify highly expressed tumor-specific target antigens for antigen-specific TCR discovery. For TCR discovery, post CIT treatment PBMCs from patient EL28 with high tumor-specific NY-ESO-1 mRNA expression were stimulated with autologous NY-ESO-1-RNA transfected APCs. Single NY-ESO-1-specific CD8⁺ FT cells were sorted by flow cytometry based on activation-induced expression of activation marker CD137 and/or IFNγ secretion for TCR cloning (FIG. 13A). Testing of cloned TCRs revealed that TCR_{CD8}-EL28-NYE #2 recognized the immune-dominant HLA-A*0201-restricted epitope NY-ESO-1₁₅₇₋₁₆₅, SLLMWITQC (Jager E. et al., 1998, J Exp Med 187 (2): 265-270) (FIG. 13B, C). TCR_{CD8}-EL28-NYE #2 was shown to mediate recognition and lysis of endogenously NY-ESO-1 expressing NSCLC (FIG. 13D, E) and melanoma cell lines (FIG. 13 F). TCR-profiling was performed with the PBMC of the patient before and after CIT demonstrating that the NY-ESO-1 reactive TCR was solely detectable after immune therapy indicating induction or expansion of the respective T-cell clonotype by CIT treatment (FIG. 13G).

### Example 6: Discovery and Characterization of Four HLA-A*0101-Restricted KRAS-Q61H-Specific TCRs from a NSCLC Patient

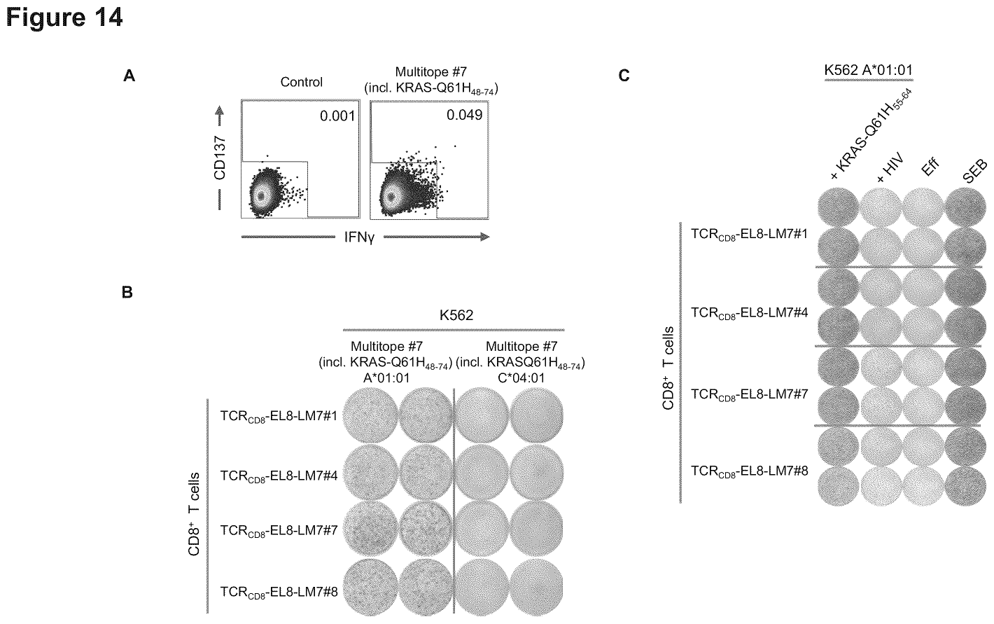

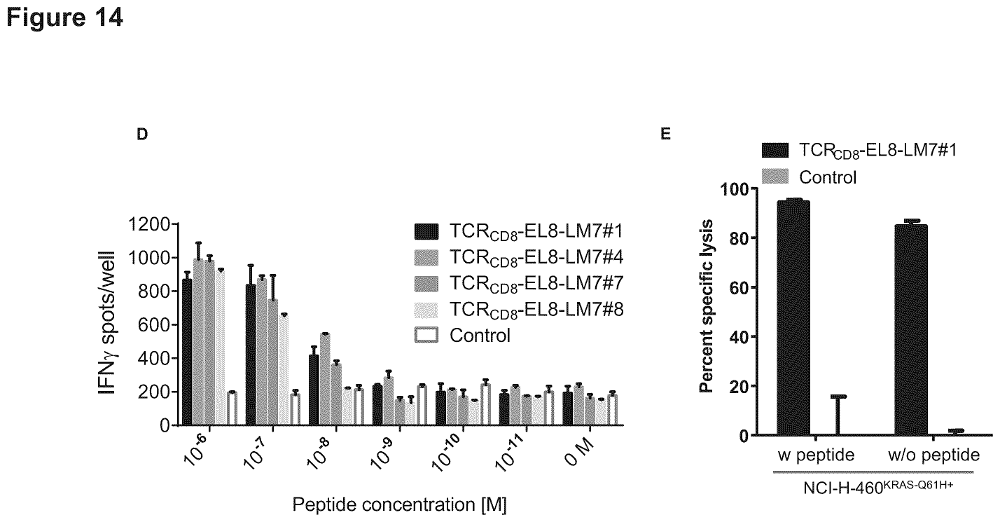

T cells derived from a NSCLC patient with high expression of the driver mutation KRAS-Q61H (Li, S., Balmain, A. & Counter, C. M., 2018, Nat Rev Cancer 18, 767-777) in his tumor were stimulated with autologous iDCs transfected with IVT-RNA encoding six linker-connected 27mer neo epitopes (mutation in position 14) including KRAS-Q61H₄₈₇₄. After single-cell sorting of reactive CD8⁺ T cells (FIG. 14A), we were able to isolate and validate four TCRs that showed reactivity against HLA-A*01:01-restricted epitope KRAS-Q61H₅₅₋₆₄, ILDTAGHEEY (FIG. 14B, C). In a next step, CD8⁺ T cells were transfected with IVT-RNA encoding the KRAS-Q61H-specific TCRs and were stimulated with K562 target cells transfected with HLA A*01:01. Target cells were further loaded with titrated amounts of KRAS-Q61H₅₅₋₆₄ target peptide (1 μm, 100 nm, 10 nm, 1 nm, 0.1 nm) to determine the functional avidity of the KRAS-Q61H₅₅₋₆₄-specific TCRs. TCR_{CD8}-EL8-LM7 #1, TCR_{CD8}-EL8-LM7 #4 and TCR_{CD8}-EL8-LM7 #7 transfected T cells showed a comparable sensitivity with a minimal recognized peptide concentration of 10 nM, whereas TCR_{CD8}-EL8-LM7 #8 demonstrated the weakest recognition of the target peptide with 100 nM (FIG. 14D). Furthermore, it was demonstrated that TCR_{CD8}-EL8-LM7 #1 mediates recognition of endogenously KRAS-Q61H expressing NSCLC cell line NCI-H-460 (FIG. 14E).

### Tables

TABLE 1
Antigen specific α/β TCRs isolated from single T cells of melanoma patients.

| | | TCR | | | | | HLA | Recognized |
|---|---|---|---|---|---|---|---|---|
| Patient | Antigen | Name | TRA | TRA CDR3 | TRB | TRB CDR3 | restriction | peptide |
| A2-09 = | NY-ESO-1 | TCRCD8- | V21*02 J20 | CAPNDYKL | V5-1 D2 J2-1 C2 | CASSFSSD | B*35:03 | NY-ESO-194-104 |
| RB0301- | | NY#99 | C | SF | | NEQFF | | |
| 02-009 | | | | | | | | |
| | NY-ESO-1 | TCRCD8- | V17 J20 C | CATDSNDY | V7-6 D1 J2-2 C2 | CASSWGGE | B*35:03 | NY-ESO-194-104 |
| | | NY#100 | | KLSF | | LFF | | |
| | NY-ESO-1 | TCRCD8- | V38-2/DV8 | CAYRSDAG | V5-4 D1 J1-3 C1 | CASSPRTG | B*35:03 | NY-ESO-194-102 |
| | | NY#101 | J52 C | GTSYGKLT | | GAGNTIYF | | |
| | | | | F | | | | |
| | NY-ESO-1 | TCRCD8- | V3 J39 C | CAVRDIEV | V5-4 D1 J2-1 C2 | CASTDRVR | B*35:03 | NY-ESO-194-104 |
| | | NY#102 | | VNAGNMLT | | YEQFF | | |
| | | | | F | | | | |
| A2-10 = | MAGE-A3 | TCRCD4- | V8-4 | CAVSDGGA | V19 D2 J2-3 C2 | CASSSGGR | DRB1*13:02 | MAGE-A3281-295 |
| RB0301- | | MA3#5 | J32*02 C | TNKLIF | | ASFTQYF | | |
| 02-010 | | | | | | | | |
| | MAGE-A3 | TCRCD4- | V9-2 J23 C | CALSDPNN | V3-1 D2 J1-1 C1 | CASSQVRG | DRB1*11:01 | MAGE-A3281-295 |
| | | MA3#9 | | QGGKLIF | | TTEAFF | | |
| | NY-ESO-1 | TCRCD4- | V24 J37 C | CAFLGSGN | V20-1*02 D2 J2-7 | CSAIRPSG | DQA1*05:05 + | n.a. |
| | | NYE#35 | | TGKLIF | C2 | GAFEQYF | DQB1*03:01 | |
| | NY-ESO-1 | TCRCD4- | V13-1 J40 | CAARATSG | V20-1*02 D1 J2-5 | CSASFSRD | DQA1*05:05 + | n.a. |
| | | NYE#36 | C | TYKYIF | C2 | IETQYF | DQB1*03:01 | |
| | Tyrosinase | TCRCD4- | V4 J23 C | CLVGPTYN | V20-1 D1 J1-4 C1 | CSARTAEV | DQA1*01:02 + | n.a. |
| | | TYR#2 | | QGGKLIF | | ELFF | DQB1*06:04 | |
| | Tyrosinase | TCRCD4- | V5 J22 C | CAVASWGS | V5-6 D2 J2-7 C2 | CASSLDAG | DQA1*01:02 + | n.a. |
| | | TYR#8 | | GSARQLTF | | YEQYF | DQB1*06:04 | |
| C2-28 = | MAGE-A3 | TCRCD8- | V39 J34 C | CAEPNTDK | V15 D1 J2-7 C2 | CATNYDRG | A*0101 | MAGE-A3168-176 |
| RB0301- | | MA3#7 | | LIF | | YEQYF | | |
| 02-028 | | | | | | | | |
| | MAGE-A3 | TCRCD8- | V12-2 J49 | CAVWATGN | V15 D1 J2-7 C2 | CATSFDRG | A*0101 | MAGE-A3168-176 |
| | | MA3#8 | C | QFYF | | YEQYF | | |
| | MAGE-A3 | TCRCD8- | V12-1 J41 | CVVNERNS | V24-1 D1 J1-5 C1 | CATSGPDS | A*0101 | MAGE-A3168-176 |
| | | MA3#10 | C | GYALNF | | NQPQHF | | |
| 53-02 = | NY-ESO-1 | TCRCD8- | V1-2 J39 C | CAVRLGDA | V9 D1 J1-1 C1 | CASSVGRG | C*03:04 | NY-ESO-196-104 |
| RB0301- | | NY#107 | | GNMLTF | | MNTEAFF | | |
| 03-002 | | | | | | | | |
| | NY-ESO-1 | TCRCD8- | V25 J39 C | CADNAGNM | V20-1*02 D1 J1-2 | CSARDPGR | B*40:01 | NY-ESO-1124-133 |
| | | NY#109 | | LTF | C1 | NYGYTF | | |
| | NY-ESO-1 | TCRCD8- | V25 J39 C | CGDNAGNM | V20-1*02 D1 J1-2 | CSARDEDR | B*40:01 | NY-ESO-1124-133 |
| | | NY#110 | | LTF | C1 | PYGYTF | | |
| C1-40 = | MAGE-A3 | TCRCD8- | V35 J32*02 | CAGQLRYG | V9*02 D1 J2-7 C2 | CASSVGGA | A*0101 | MAGE-A3168-176 |
| RB0301- | | MA3#1 | C | GATNKLIF | | SYEQYF | | |
| 01-040 | | | | | | | | |

V: variable,
D: diversity,
J: joining,
C: constant,
TRA: T cell receptor alpha,
TRB: T cell receptor beta,
CDR3: Complementarity determining region 3.
IMGT nomenclature.

TABLE 2
Antigen specific α/β TCRs isolated from single T cells of NSCLC patients.

| | | | | | | | HLA | Recognized |
|---|---|---|---|---|---|---|---|---|
| Patient | Antigen | TCR Name | TRA | TRA CDR3 | TRB | TRB CDR3 | restriction | peptide |
| EL28 | NY-ESO- | TCRCD8-EL28- | V34 J13 | CGADMLDY | V12-4D1J2- | CASRRGGS | A*02:01 | NY-ESO- |
| (CIT) | 1 | NY#2 | C | QKVTF | 7C2 | EQYF | | 1₁₅₇₋₁₆₅ |
| EL8 | KRAS- | TCRCD8-EL8- | V14/DV4 | CAMREGQD | V9 D1 J1-1 | CASSVLGP | A*01:01 | KRAS- |
| | Q61H | LM7#1 | J12 C | SSYKLIF | C1 | NTEAFF | | Q61H₅₅₋₆₄ |
| | KRAS- | TCRCD8-EL8- | V19 J52 | CALSNAGG | V27 J1-2 | CASSRRLP | A*01:01 | KRAS- |
| | Q61H | LM7#4 | C | TSYGKLTF | C1 | PPYGYTF | | Q61H₅₅₋₆₄ |
| | KRAS- | TCRCD8-EL8- | V19 | CALSESSN | V27 D2 J1-2 | CASSRREP | A*01:01 | KRAS- |
| | Q61H | LM7#7 | J37*02 C | TGKLIF | C1 | PPYGYTF | | Q61H₅₅₋₆₄ |
| | KRAS- | TCRCD8-EL8- | V14/DV4 | CAMREIRD | V9 D1 J1-1 | CASSPAGP | A*01:01 | KRAS- |
| | Q61H | LM7#8 | J12 C | SSYKLIF | C1 | NTEAFF | | Q61H₅₅₋₆₄ |

V: variable,
D: diversity,
J: joining,
C: constant,
TRA: T cell receptor alpha,
TRB: T cell receptor beta,
CDR3: Complementarity determining region 3.
IMGT nomenclature.
CIT: Checkpoint inhibitor treated.

In the following, the T cell receptor sequences obtained are shown. The underlined sequences are the CDR sequences, wherein the first sequence in each T cell receptor chain is CDR1, followed by CDR2 and CDR3.

Respective TCR chain VDJ region nucleotide and amino acid sequences

SEQ ID NO: 5; >hTCR-CD8-RB0301-02-009-NYE#99a1_V21*02J20C_CAPNDYKLSF
METLLGLLILWLQLQWVSSKQEVTQIPAALSVPEGENLVLNCSFTDSAIYNLQWFRQDPGKGLTSLLLIQSSQREQTSGRLNAS
LDKSSGRSTLYIAASQPGDSATYLCAPNDYKLSFGAGTTVTVRA [symbol]
SEQ ID NO: 6; >hTCR-CD8-RB0301-02-009-NYE#99b_V5-1D2J2-1C2_CASSFSSDNEQFF_aa
exchange S>C
MGCRLLCWVLLCLLGAGPVKAGVTQTPRYLIKTRGQQVTLSCSPISGHRSVSWYQQTPGQGLQFLFEYFSETQRNKGNFPGRFS
GRQFSNSRSEMNVSTLELGDSALYLCASSFSSDNEQFFGPGTRLTVL [symbol]
SEQ ID NO: 7; >hTCR-CD8-RB0301-02-009-NYE#100a_V17J20C_CATDSNDYKLSF
METLLGVSLVILWLQLARVNSQQGEEDPQALSIQEGENATMNCSYKTSINNLQWYRQNSGRGLVHLILIRSNEREKHSGRLRVT
LDTSKKSSSLLITASRAADTASYFCATDSNDYKLSFGAGTTVTVRA [symbol]
SEQ ID NO: 8; >hTCR-CD8-RB0301-02-009-NYE#100b_V7-6D1J2-2C2_CASSWGGELFF_aa
exchange GTS>DIW
MDIWLLCWVVLGFLGTDHTGAGVSQSPRYKVTKRGQDVALRCDPISGHVSLYWYRQALGQGPEFLTYFNYEAQQDKSGLPNDRF
SAERPEGSISTLTIQRTEQRDSAMYRCASSWGGELFFGEGSRLTVL [symbol]
SEQ ID NO: 9; >hTCR-CD8-RB0301-02-009-NYE#101a1_V38-2/DV8J52C_CAYRSDAGGTSYGKLTF
MACPGFLWALVISTCLEFSMAQTVTQSQPEMSVQEAETVTLSCTYDTSESDYYLFWYKQPPSRQMILVIRQEAYKQQNATENRF
SVNFQKAAKSFSLKISDSQLGDAAMYFCAYRSDAGGTSYGKLTFGQGTILTVHP [symbol]
SEQ ID NO: 10; >hTCR-CD8-RB0301-02-009-NYE#101b_V5-4D1J1-
3C1_CASSPRTGGAGNTIYF_aa exchanges GP>SI
MSIGLLCWVLLCLLGAGSVETGVTQSPTHLIKTRGQQVTLRCSSQSGHNTVSWYQQALGQGPQFIFQYYREEENGRGNFPPRFS
GLQFPNYSSELNVNALELDDSALYLCASSPRTGGAGNTIYFGEGSWLTVV [symbol]
SEQ ID NO: 11; >hTCR-CD8-RB0301-02-009-NYE#102a_V3J39C_CAVRDIEVVNAGNMLTF
MASAPISMLAMLFTLSGLRAQSVAQPEDQVNVAEGNPLTVKCTYSVSGNPYLFWYVQYPNRGLQFLLKYITGDNLVKGSYGFEA
EFNKSQTSFHLKKPSALVSDSALYFCAVRDIEVVNAGNMLTFGGGTRLMVKP [symbol]
SEQ ID NO: 12; >hTCR-CD8-RB0301-02-009-NYE#102b_V5-4D1J2-1C2_CASTDRVRYEQFF_aa
exchanges GPG>DIW, L>V
MDIWLVCWVLLCLLGAGSVETGVTQSPTHLIKTRGQQVTLRCSSQSGHNTVSWYQQALGQGPQFIFQYYREEENGRGNFPPRFS
GLQFPNYSSELNVNALELDDSALYLCASTDRVRYEQFFGPGTRLTVL [symbol]
SEQ ID NO: 23; >hTCR-CD4-RB0301-02-010-MA3#5a_V8-4J32*02C_CAVSDGGATNKLIF
MLLLLVPVLEVIFTLGGTRAQSVTQLGSHVSVSEGALVLLRCNYSSSVPPYLFWYVQYPNQGLQLLLKYTSAATLVKGINGFEA
EFKKSETSFHLTKPSAHMSDAAEYFCAVSDGGATNKLIFGTGTLLAVQP [symbol]
SEQ ID NO: 24; >hTCR-CD4-RB0301-02-010-MA3#5b_V19D2J2-
3C2_CASSSGGRASFTQYF_aa exchanges SNQV>GTRL, F>L
MGTRLLCCVVLCLLGANTVDGGITQSPKYLFRKEGQNVTLSCEQNLNHDAMYWYRQDPGQGLRLIYYSQIVNDFQKGDIAEGYS
VSREKKESFPLTVTSAQKNPTAFYLCASSSGGRASFTQYFGPGTRLTVL [symbol]
SEQ ID NO: 25; >hTCR-CD4-RB0301-02-010-MA3#9a_V9-2J23C_CALSDPNNQGGKLIF
MNYSPGLVSLILLLLGRTRGNSVTQMEGPVTLSEEAFLTINCTYTATGYPSLFWYVQYPGEGLQLLLKATKADDKGSNKGFEAT
YRKETTSFHLEKGSVQVSDSAVYFCALSDPNNOGGKLIFGQGTELSVKP [symbol]
SEQ ID NO: 26; >hTCR-CD4-RB0301-02-010-MA3#9b_V3-1D2J1-
1C1_CASSQVRGTTEAFF_aa exchange C>T
MGTRLLCCVVFCLLQAGPLDTAVSQTPKYLVTQMGNDKSIKCEQNLGHDTMYWYKQDSKKFLKIMFSYNNKELIINETVPNRFS
PKSPDKAHLNLHINSLELGDSAVYFCASSQVRGTTEAFFGQGTRLTVV [symbol]
SEQ ID NO: 13; >hTCR-CD4-RB0301-02-010-NYE#35a_V24J37C_CAFLGSGNTGKLIF
MEKNPLAAPLLILWFHLDCVSSILNVEQSPQSLHVQEGDSTNFTCSFPSSNFYALHWYRWETAKSPEALFVMTLNGDEKKKGRI
SATLNTKEGYSYLYIKGSQPEDSATYLCAFLGSGNTGKLIFGQGTTLQVK [symbol]
SEQ ID NO: 14; >hTCR-CD4-RB0301-02-010-NYE#35b_V20-1*02D2J2-
7C2_CSAIRPSGGAFEQYF
MLLLLLLLGPGSGLGAVVSQHPSWVICKSGTSVKIECRSLDFQATTMFWYRQFPKQSLMLMATSNEGSKATYEQGVEKDKFLIN
HASLTLSTLTVTSAHPEDSSFYICSAIRPSGGAFEQYFGPGTRLTVT [symbol]
SEQ ID NO: 15; >hTCR-CD4-RB0301-02-010-NYE#36a_V13-1J40C_CAARATSGTYKYIF
MTSIRAVFIFLWLQLDLVNGENVEQHPSTLSVQEGDSAVIKCTYSDSASNYFPWYKQELGKGPQLIIDIRSNVGEKKDQRIAVT
LNKTAKHFSLHITETQPEDSAVYFCAARATSGTYKYIFGTGTRLKVLA [symbol]
SEQ ID NO: 16; >hTCR-CD4-RB0301-02-010-NYE#36b_V20-1*02D1J2-
5C2_CSASFSRDIETQYF
MLLLLLLLGPGSGLGAVVSQHPSRVICKSGTSVKIECRSLDFQATTMFWYRQFPKKSLMLMATSNEGSKATYEQGVEKDKFLIN
HASLTLSTLTVTSAHPEDSSFYICSASFSRDIETQYFGPGTRLLVL [symbol]
SEQ ID NO: 35; >hTCR-CD4-RB0301-02-010-TYR#2a_V4J23C_CLVGPTYNQGGKLIF
MRQVARVIVFLTLSTLSLAKTTQPISMDSYEGQEVNITCSHNNIATNDYITWYQQFPSQGPRFIIQGYKTKVTNEVASLFIPAD
RKSSTLSLPRVSLSDTAVYYCLVGPTYNQGGKLIFGQGTELSVKP [symbol]
SEQ ID NO: 36; >hTCR-CD4-RB0301-02-010-TYR#2b_V20-1*01D1J1-4C1_CSARTAEVELFF
MLLLLLLLGPGSGLGAVVSQHPSRVICKSGTSVKIECRSLDFQATTMFWYRQFPKKSLMLMATSNEGSKATYEQGVEKDKFLIN
HASLTLSTLTVTSAHPEDSSFYICSARTAEVELFFGSGTQLSVL [symbol]
SEQ ID NO: 37; >hTCR-CD4-RB0301-02-010-TYR#8a2_V5J22C_CAVASWGSGSARQLTF
MKTFAGFSFLFLWLQLDCMSRGEDVEQSLFLSVREGDSSVINCTYTDSSSTYLYWYKQEPGAGLQLLTYIFSNMDMKQDQRLTV
LLNKKDKHLSLRIADTQTGDSAIYFCAVASWGSGSARQLTFGSGTQLTVLP [symbol]
SEQ ID NO: 38; >hTCR-CD4-RB0301-02-010-TYR#8b_V5-6D2J2-
7C2_CASSLDAGYEQYF_aa exchanges PG>TR
MGTRLLCWALLCLLGAGLVDAGVTQSPTHLIKTRGQQVTLRCSPKSGHDTVSWYQQALGQGPQFIFQYYEEEERQRGNFPDRFS
GHQFPNYSSELNVNALLLGDSALYLCASSLDAGYEQYFGPGTRLTVT [symbol]
SEQ ID NO: 27; >hTCR-CD8-RB0301-02-028-MA3#7a_V39J34C_CAEPNTDKLIF
MKKLLAMILWLQLDRLSGELKVEQNPLFLSMQEGKNYTIYCNYSTTSDRLYWYRQDPGKSLESLFVLLSNGAVKQEGRLMASLD
TKARLSTLHITAAVHDLSATYFCAEPNTDKLIFGTGTRLQVFP [symbol]
SEQ ID NO: 28; >hTCR-CD8-RB0301-02-028-MA3#7b_V15D1J2-7C2_CATNYDRGYEQYF
MGPGLLHWMALCLLGTGHGDAMVIQNPRYQVTQFGKPVTLSCSQTLNHNVMYWYQQKSSQAPKLLFHYYDKDFNNEADTPDNFQ
SRRPNTSFCFLDIRSPGLGDTAMYLCATNYDRGYEQYFGPGTRLTVT [symbol]
SEQ ID NO: 29; >hTCR-CD8-RB0301-02-028-MA3#8a_V12-2J49C_CAVWATGNQFYF
MKSLRVLLVILWLQLSWVWSQQKEVEQNSGPLSVPEGAIASLNCTYSDRGSQSFFWYRQYSGKSPELIMFIYSNGDKEDGRFTA
QLNKASQYVSLLIRDSQPSDSATYLCAVWATGNQFYFGTGTSLTVIP [symbol]
SEQ ID NO: 30; >hTCR-CD8-RB0301-02-028-MA3#8b_V15D1J2-7C2_CATSFDRGYEQYF
MGPGLLHWMALCLLGTGHGDAMVIQNPRYQVTQFGKPVTLSCSQTLNHNVMYWYQQKSSQAPKLLFHYYDKDFNNEADTPDNFQ
SRRPNTSFCFLDIRSPGLGDTAMYLCATSFDRGYEQYFGPGTRLTVT [symbol]
SEQ ID NO: 31; >hTCR-CD8-RB0301-02-028-MA3#10a_V12-1J41C_CVVNERNSGYALNF
MISLRVLLVILWLQLSWVWSQRKEVEQDPGPFNVPEGATVAFNCTYSNSASQSFFWYRQDCRKEPKLLMSVYSSGNEDGRFTAQ
LNRASQYISLLIRDSKLSDSATYLCVVNERNSGYALNFGKGTSLLVTP [symbol]
SEQ ID NO: 32; >hTCR-CD8-RB0301-02-028-MA3#10B_V24-1D1J1-5C1_CATSGPDSNQPQHF
MASLLFFCGAFYLLGTGSMDADVTQTPRNRITKTGKRIMLECSQTKGHDRMYWYRQDPGLGLRLIYYSFDVKDINKGEISDGYS
VSRQAQAKFSLSLESAIPNQTALYFCATSGPDSNQPQHFGDGTRLSIL [symbol]
SEQ ID NO: 17; >hTCR-CD8-RB0301-03-002-NYE#107a2 formerly
NYE#113a_V1-2J39C_CAVRLGDAGNMLTF_aa exchange V>A
MWGAFLLYVSMKMGGTTGQNIDQPTEMTATEGAIVQINCTYQTSGFNGLFWYQQHAGEAPTFLSYNVLDGLEEKGRFSSFLSRS
KGYSYLLLKELQMKDSASYLCAVRLGDAGNMLTEGGGTRLMVKP [symbol]
SEQ ID NO: 18; >hTCR-CD8-RB0301-03-002-NYE#107b_V9D1J1-
1C1_CASSVGRGMNTEAFF_aa exchange F>T
MGTRLLCCVAFCLLGAGPVDSGVTQTPKHLITATGQRVTLRCSPRSGDLSVYWYQQSLDQGLQFLIQYYNGEERAKGNILERFS
AQQFPDLHSELNLSSLELGDSALYFCASSVGRGMNTEAFFGQGTRLTVV [symbol]
SEQ ID NO: 19; >hTCR-CD8-RB0301-03-002-NYE#109a_V25J39C_CADNAGNMLTF
MLLITSMLVLWMQLSQVNGQQVMQIPQYQHVQEGEDFTTYCNSSTTLSNIQWYKQRPGGHPVFLIQLVKSGEVKKQKRLTFQFG
EAKKNSSLHITATQTTDVGTYFCADNAGNMLTFGGGTRLMVKP [symbol]
SEQ ID NO: 20; >hTCR-CD8-RB0301-03-002-NYE#109b_V20-1*02D1J1-
2C1_CSARDPGRNYGYTF_1 aa insertion +A
MLLLLLLLGPAGSGLGAVVSQHPSWVICKSGTSVKIECRSLDFQATTMFWYRQFPKQSLMLMATSNEGSKATYEQGVEKDKFLI
NHASLTLSTLTVTSAHPEDSSFYICSARDPGRNYGYTFGSGTRLTVV [symbol]
SEQ ID NO: 21; >hTCR-CD8-RB0301-03-002-NYE#110a_V25J39C_CGDNAGNMLTF
MLLITSMLVLWMQLSQVNGQQVMQIPQYQHVQEGEDFTTYCNSSTTLSNIQWYKQRPGGHPVFLIQLVKSGEVKKQKRLTFQFG
EAKKNSSLHITATQTTDVGTYFCGDNAGNMLTFGGGTRLMVKP [symbol]
SEQ ID NO: 22; >hTCR-CD8-RB0301-03-002-NYE#110b_V20-1*02D1J1-2C1_CSARDEDRPYGYTF
MLLLLLLLGPGSGLGAVVSQHPSWVICKSGTSVKIECRSLDFQATTMFWYRQFPKQSLMLMATSNEGSKATYEQGVEKDKFLIN
HASLTLSTLTVTSAHPEDSSFYICSARDEDRPYGYTFGSGTRLTVV [symbol]
SEQ ID NO: 33; >hTCR-CD8-RB0301-01-040-MA3#1a_V35J32*02C_CAGQLRYGGATNKLIF
MLLEHLLIILWMQLTWVSGQQLNQSPQSMFIQEGEDVSMNCTSSSIFNTWLWYKQEPGEGPVLLIALYKAGELTSNGRLTAQFG
ITRKDSFLNISASIPSDVGIYFCAGQLRYGGATNKLIFGTGTLLAVQP [symbol]
SEQ ID NO: 34; >hTCR-CD8-RB0301-01-040-MA3#1b_V9*02D1J2-
7C2_CASSVGGASYEQYF_aa exchange F>C
MGCRLLCCVAFCLLGAGPVDSGVTQTPKHLITATGQRVTLRCSPRSGDLSVYWYQQSLDQGLQFLIHYYNGEERAKGNILERFS
AQQFPDLHSELNLSSLELGDSALYFCASSVGGASYEQYFGPGTRLTVT [symbol]
Yellow, underlined = CDR regions,
Bold, italic = Begin of constant region

Regarding the TRBV20-1 gene, the ENSEML references were used (TRBV20-1*01 ENST00000390394; TRBV20-1*02 ENST00000633466) since the reference sequences from the IMGT database contain incorrect insertions in the Leader sequence!

Respective TCR chain VDJ region nucleotide and amino acid sequences

Patient EL000028 CTAG1A (=NY-ESO-I) TCR:
SEQ ID NO: 91; >hTCR-CD8-EL28-NYE#2a_V34J13C_CGADMLDYQKVTF_aa
METVLQVLLGILGFQAAWVSSQELEQSPQSLIVQEGKNLTINCTSSKTLYGLYWYKQKYGEGLIFLMMLQKGGEEKSHEKIT
AKLDEKKQQSSLHITASQPSHAGIYLCGADMLDYOKVTFGIGTKLQVIP [symbol]
SEQ ID NO: 92; >hTCR-CD8-EL28-NYE#2b_V12-4D1J2-7C2_CASRRGGSEQYF_aa
MGSWTLCCVSLCILVAKHTDAGVIQSPRHEVTEMGQEVTLRCKPISGHDYLFWYRQTMMRGLELLIYFNNNVPIDDSGMPED
RFSAKMPNASFSTLKIQPSEPRDSAVYFCASRRGGSEQYFGPGTRLTVT [symbol]
Patient EL000008 KRAS-Q61H TCRs:
SEQ ID NO: 93; >hTCR-CD8-EL08-LM7#1a_V14/DV4J12C_CAMREGQDSSYKLIF_aa
MSLSSLLKVVTASLWLGPGIAQKITQTQPGMFVQEKEAVTLDCTYDTSDPSYGLFWYKQPSSGEMIFLIYQGSYDQQNATEG
RYSLNFQKARKSANLVISASQLGDSAMYFCAMREGQDSSYKLIFGSGTRLLVRP [symbol]
SEQ ID NO: 94; >hTCR-CD8-EL08-LM7#1b_V9D1J1-1C1_CASSVLGPNTEAFF_aa
MGTRLLCCVAFCLLGAGPVDSGVTQTPKHLITATGQRVTLRCSPRSGDLSVYWYQQSLDQGLQFLIQYYNGEERAKGNILER
FSAQQFPDLHSELNLSSLELGDSALYFCASSVLGPNTEAFFGQGTRLTVV [symbol]
SEQ ID NO: 95; >hTCR-CD8-EL08-LM7#4a_V19J52C_CALSNAGGTSYGKLTF_aa
MLTASLLRAVIASICVVSSMAQKVTQAQTEISVVEKEDVTLDCVYETRDTTYYLFWYKQPPSGELVFLIRRNSFDEQNEISG
RYSWNFQKSTSSFNFTITASQVVDSAVYFCALSNAGGTSYGKLTFGQGTILTVHP [symbol]
SEQ ID NO: 96; >hTCR-CD8-EL08-LM7#4b_V27J1-2C1_CASSRRLPPPYGYTF_aa
MGPQLLGYVVLCLLGAGPLEAQVTQNPRYLITVTGKKLTVTCSQNMNHEYMSWYRQDPGLGLRQIYYSMNVEVTDKGDVPEG
YKVSRKEKRNFPLILESPSPNQTSLYFCASSRRLPPPYGYTFGSGTRLTVV [symbol]
SEQ ID NO: 97; >hTCR-CD8-EL08-LM7#7a_V19J37*02C_CALSESSNTGKLIF_aa
MLTASLLRAVIASICVVSSMAQKVTQAQTEISVVEKEDVTLDCVYETRDTTYYLFWYKQPPSGELVFLIRRNSFDEQNEISG
RYSWNFQKSTSSFNFTITASQVVDSAVYFCALSESSNTGKLIFGQGTTLQVKP [symbol]
SEQ ID NO: 98; >hTCR-CD8-EL08-LM7#7b_V27D2J1-2C1_CASSRREPPPYGYTF_aa
MGPQLLGYVVLCLLGAGPLEAQVTQNPRYLITVTGKKLTVTCSQNMNHEYMSWYRQDPGLGLRQIYYSMNVEVTDKGDVPEG
YKVSRKEKRNFPLILESPSPNQTSLYFCASSRREPPPYGYTFGSGTRLTVV [symbol]
SEQ ID NO: 99; >hTCR-CD8-EL08-LM7#8a_V14/DV4J12C_CAMREIRDSSYKLIF_aa
MSLSSLLKVVTASLWLGPGIAQKITQTQPGMFVQEKEAVTLDCTYDTSDPSYGLFWYKQPSSGEMIFLIYQGSYDQQNATEG
RYSLNFQKARKSANLVISASQLGDSAMYFCAMREIRDSSYKLIFGSGTRLLVRP [symbol]
SEQ ID NO: 100; >hTCR-CD8-EL08-LM7#8b_V9D1J1-1C1_CASSPAGPNTEAFF_aa_aa exchange Y>H
MGTRLLCCVAFCLLGAGPVDSGVTQTPKHLITATGQRVTLRCSPRSGDLSVYWYQQSLDQGLQFLIQHYNGEERAKGNILER
FSAQQFPDLHSELNLSSLELGDSALYFCASSPAGPNTEAFFGQGTRLTVV [symbol]
Yellow, underlined = CDR regions,
Bold, italic = Begin of constant region

1. A peptide comprising an amino acid sequence selected from the group consisting of SEQ ID NOs: 39 to 44, 101, and 102 or a variant of said amino acid sequence.

2.-7. (canceled)

8. An immune effector cell which is reactive with a peptide of claim 1.

9. A T cell receptor polypeptide or a T cell receptor selected from the group consisting of:
(I) a T cell receptor which is reactive with a peptide of claim 1, or a polypeptide chain of said T cell receptor;
(II) a T cell receptor polypeptide or a T cell receptor comprising said T cell receptor polypeptide, wherein said T cell receptor polypeptide is selected from the group consisting of:
(i) a T cell receptor polypeptide comprising at least one, preferably two, more preferably all three of the CDR sequences of a T cell receptor α-chain selected from SEQ ID NOs: 5, 7, 9, 11, 13, 15, 17, 19, 21, 23, 25, 27, 29, 31, 33, 35, 37, 91, 93, 95, 97, and 99 or a variant thereof, and
(ii) a T cell receptor polypeptide comprising a T cell receptor α-chain sequence selected from SEQ ID NOs: 5, 7, 9, 11, 13, 15, 17, 19, 21, 23, 25, 27, 29, 31, 33, 35, 37, 91, 93, 95, 97, and 99 or a variant thereof,
(III) a T cell receptor polypeptide or a T cell receptor comprising said T cell receptor polypeptide, wherein said T cell receptor polypeptide is selected from the group consisting of:
(i) a T cell receptor polypeptide comprising at least one, preferably two, more preferably all three of the CDR sequences of a T cell receptor β-chain selected from SEQ ID NOs: 6, 8, 10, 12, 14, 16, 18, 20, 22, 24, 26, 28, 30, 32, 34, 36, 38, 92, 94, 96, 98, and 100 or a variant thereof, and
(ii) a T cell receptor polypeptide comprising a T cell receptor β-chain sequence selected from SEQ ID NOs: 6, 8, 10, 12, 14, 16, 18, 20, 22, 24, 26, 28, 30, 32, 34, 36, 38, 92, 94, 96, 98, and 100 or a variant thereof; and
(V) a T cell receptor selected from the group consisting of:
(i) a T cell receptor comprising:
(a) at least one, preferably two, more preferably all three of the CDR sequences of the T cell receptor α-chain of SEQ ID NO: x or a variant thereof; and
(b) at least one, preferably two, more preferably all three of the CDR sequences of a T cell receptor β-chain of SEQ ID NO: x+1 or a variant thereof;
wherein x is selected from 5, 7, 9, 11, 13, 15, 17, 19, 21, 23, 25, 27, 29, 31, 33, 35, 37, 91, 93, 95, 97, and 99;
and
(ii) a T cell receptor comprising:
(a) the T cell receptor α-chain sequence of SEQ ID NO: x or a variant thereof; and
(b) the T cell receptor β-chain sequence of SEQ ID NO: x+1 or a variant thereof;
wherein x is selected from 5, 7, 9, 11, 13, 15, 17, 19, 21, 23, 25, 27, 29, 31, 33, 35, 37, 91, 93, 95, 97, and 99.

10-12. (canceled)

13. A nucleic acid encoding the T cell receptor chain polypeptide or T cell receptor of claim 9.

14. A cell which is genetically modified to express the T cell receptor polypeptide or T cell receptor of claim 9.

15. (canceled)

16. (canceled)

17. A method for preparing immune effector cells genetically modified to express the T cell receptor of claim 9, comprising delivering nucleic acid encoding the T cell receptor to the immune effector cells.

18. The method of claim 17, which comprises contacting the immune effector cells with particles comprising the nucleic acid, wherein optionally said particles further comprise a targeting molecule for targeting the immune effector cells and/or wherein contacting the immune effector cells with the particles delivers the nucleic acid to the immune effector cells.

19-20. (canceled)

21. The method of claim 17, wherein the immune effector cells to be genetically modified are present in vivo or in vitro, wherein, if the immune effector cells to be genetically modified are present in vivo in a subject, the method optionally comprises administering the particles to the subject.

22. (canceled)

23. A pharmaceutical composition comprising:
the peptide of claim 1.

24. A method for treating a subject, comprising administering the pharmaceutical composition of claim 23 to the subject.

25. (canceled)

26. The method of claim 24 which is a method of inducing an immune response in said subject, wherein optionally the immune response is a T cell-mediated immune response and/or wherein the immune response is an immune response to a target cell population or target tissue expressing an antigen, which target cell population or target tissue optionally is cancer cells, cancer tissue or solid cancer.

27-30. (canceled)

31. A method for treating a subject having a disease, disorder or condition associated with expression or elevated expression of an antigen comprising providing immune effector cells genetically modified to express the T cell receptor of claim 9, the T cell receptor being targeted to the antigen associated with the disease, disorder or condition or cells expressing the antigen associated with the disease, disorder or condition, to the subject.

32. The method of claim 31, wherein the disease, disorder or condition is cancer, optionally solid cancer, and the antigen associated with the disease, disorder or condition is a tumor antigen.

33. (canceled)

34. The method of claim 31, wherein the immune effector cells genetically modified to express the T cell receptor:
(I) are provided to the subject by administering the immune effector cells genetically modified to express the T cell receptor;
(II) by generating the immune effector cells genetically modified to express the T cell receptor in the subject: or
(III) are prepared by a method comprising delivering nucleic acid encoding the T cell receptor to the immune effector cells.

35. (canceled)

36. A method for treating a subject having a disease, disorder or condition associated with expression or elevated expression of an antigen comprising providing immune effector cells genetically modified to express a T cell receptor, the T cell receptor being targeted to the antigen associated with the disease, disorder or condition or cells expressing the antigen associated with the disease, disorder or condition, to the subject, wherein the immune effector cells genetically modified to express the T cell receptor are prepared by the method according to claim 17.

37-40. (canceled)

41. The method of claim 24, which is a method for treating or preventing cancer in a subject, wherein optionally the cancer is solid cancer and/or is associated with expression or elevated expression of a tumor antigen targeted by the T cell receptor.

42-43. (canceled)

44. The method of claim 24, which further comprises administering to the subject an antigen targeted by the T cell receptor, a polynucleotide encoding the antigen, or a host cell genetically modified to express the antigen, wherein optionally the polynucleotide encoding the antigen is RNA and/or the host cell genetically modified to express the antigen comprises a polynucleotide encoding the antigen.

45-46. (canceled)

47. The method of claim 17, wherein the immune effector cells genetically modified to express the T cell receptor comprise a DNA or RNA polynucleotide encoding the T cell receptor and wherein the genetic modification is transient or stable.

48-50. (canceled)

51. The method of claim 17, wherein the genetic modification takes place by a virus-based method, transposon-based method, or a gene editing-based method, wherein preferably the gene editing-based method involves CRISPR-based gene editing.

52. (canceled)

53. The method of claim 18, wherein the particles are non-viral particles, lipid-based particles, polymer-based particles, lipid- and polymer-based particles or nanoparticles, wherein optionally:
(I) the particles are functionalized with a targeting molecule on their surface;
(II) the particles are functionalized with a targeting molecule by linking the targeting molecule to at least one particle-forming component and/or (III) the targeting molecule targets CD8, CD4 or CD3.

54-58. (canceled)

59. The method of claim 17, wherein the immune effector cells are T cells, preferably CD4+ or CD8+ T cells.

60. (canceled)

61. The cell of claim 14, wherein the cell is an antigen presenting cell or an immune effector cell.

62. A cell comprising nucleic acid encoding the T cell receptor polypeptide or T cell receptor of claim 9.

63. A nucleic acid encoding the peptide of claim 1.

64. A cell which is genetically modified to express the peptide of claim 1.

65. The cell of claim 64, wherein the cell is an antigen presenting cell or an immune effector cell.

66. A pharmaceutical composition comprising the nucleic acid of claim 13.

67. A pharmaceutical composition comprising the cell of claim 14.

68. A pharmaceutical composition comprising the immune effector cell of claim 8.


SEQUENCE LISTING (112 sequences)

SEQ ID NO: 1 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 3 - protein, 529 aa, Homo sapiens [sequence omitted]

SEQ ID NO: 4 - protein, 551 aa, Homo sapiens
MNESPDPTDLAGVIIELGPNDSPQTSEFKGATEEAPAKESPHTSEFKGAARVSPISESVLARLSKFEVEDAENVASYDSKIKKIVHSIVSSFAFGLFGVFLVLLDVTLILADLIFTDSKLYIPLEYRSISLAIALFFLMDVLLRVFVERRQQYFSDLFNILDTAIIVILLLVDVVYIFFDIKLLRNIPRWTHLLRLLRLIILLRIFHLFHQKRQLEKLIRRRVSENKRRYTRDGFDLDLTYVTERIIAMSFPSSGRQSFYRNPIKEVVRFLDKKHRNHYRVYNLCSERAYDPKHFHNRVVRIMIDDHNVPTLHQMVVFTKEVNEWMAQDLENIVAIHCKGGTDRTGTMVCAFLIASEICSTAKESLYYFGERRTDKTHSEKFQGVETPSQKRYVAYFAQVKHLYNWNLPPRRILFIKHFIIYSIPRYVRDLKIQIEMEKKVVFSTISLGKCSVLDNITTDKILIDVFDGLPLYDDVKVQFFYSNLPTYYDNCSFYFWLHTSFIENNRLYLPKNELDNLHKQKARRIYPSDFAVEILFGEKMTSSDVVAGSD

SEQ ID NO: 5 - protein, 143 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAPNDYKLSFGAGTTVTVRANIQNPDPAVYQLRDS

SEQ ID NO: 6 - protein, 146 aa, Homo sapiens
MGCRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFSSDNEQFFGPGTRLTVLEDLKNVFPPEVAVFE

SEQ ID NO: 7 - protein, 145 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDSNDYKLSFGAGTTVTVRANIQNPDPAVYQLRDS

SEQ ID NO: 8 - protein, 145 aa, Homo sapiens
MDIWLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSWGGELFFGEGSRLTVLEDLKNVFPPEVAVFE

SEQ ID NO: 9 - protein, 153 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDAGGTSYGKLTFGQGTILTVHPNIQNPDPAVYQLRDS

SEQ ID NO: 10 - protein, 149 aa, Homo sapiens
MSIGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSPRTGGAGNTIYFGEGSWLTVVEDLNKVFPPEVAVFE

SEQ ID NO: 11 - protein, 151 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDIEVVNAGNMLTFGGGTRLMVKPHIQNPDPAVYQLRDS

SEQ ID NO: 12 - protein, 146 aa, Homo sapiens
MDIWLVCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASTDRVRYEQFFGPGTRLTVLEDLKNVFPPEVAVFE

SEQ ID NO: 13 - protein, 150 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFLGSGNTGKLIFGQGTTLQVKPDIQNPDPAVYQLRDS

SEQ ID NO: 14 - protein, 146 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAIRPSGGAFEQYFGPGTRLTVTEDLKNVFPPEVAVFE

SEQ ID NO: 15 - protein, 147 aa, Homo sapiens
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAARATSGTYKYIFGTGTRLKVLANIQNPDPAVYQLRDS

SEQ ID NO: 16 - protein, 145 aa, Homo sapiens
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSASFSRDIETQYFGPGTRLLVLEDLKNVFPPEVAVFE

SEQ ID NO: 17 - protein, 143 aa, Homo sapiens
MWGAFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRLGDAGNMLTFGGGTRLMVKPHIQNPDPAVYQLRDS

SEQ ID NO: 18 - protein, 148 aa, Homo sapiens
MGTRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGRGMNTEAFFGQGTRLTVVEDLNKVFPPEVAVFE

SEQ ID NO: 19 - protein, 142 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCADNAGNMLTFGGGTRLMVKPHIQNPDPAVYQLRDS

SEQ ID NO: 20 - protein, 146 aa, Homo sapiens
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDPGRNYGYTFGSGTRLTVVEDLNKVFPPEVAVFE

SEQ ID NO: 21 - protein, 142 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCGDNAGNMLTFGGGTRLMVKPHIQNPDPAVYQLRDS

SEQ ID NO: 22 - protein, 145 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDEDRPYGYTFGSGTRLTVVEDLNKVFPPEVAVFE

SEQ ID NO: 23 - protein, 148 aa, Homo sapiens
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSDGGATNKLIFGTGTLLAVQPNIQNPDPAVYQLRDS

SEQ ID NO: 24 - protein, 148 aa, Homo sapiens
MGTRLLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSGGRASFTQYFGPGTRLTVLEDLKNVFPPEVAVFE

SEQ ID NO: 25 - protein, 148 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSDPNNQGGKLIFGQGTELSVKPNIQNPDPAVYQLRDS

SEQ ID NO: 26 - protein, 147 aa, Homo sapiens
MGTRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQVRGTTEAFFGQGTRLTVVEDLNKVFPPEVAVFE

SEQ ID NO: 27 - protein, 142 aa, Homo sapiens
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAEPNTDKLIFGTGTRLQVFPNIQNPDPAVYQLRDS

SEQ ID NO: 28 - protein, 146 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATNYDRGYEQYFGPGTRLTVTEDLKNVFPPEVAVFE

SEQ ID NO: 29 - protein, 146 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVWATGNQFYFGTGTSLTVIPNIQNPDPAVYQLRDS

SEQ ID NO: 30 - protein, 146 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDTAMYLCATSFDRGYEQYFGPGTRLTVTEDLKNVFPPEVAVFE

SEQ ID NO: 31 - protein, 147 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNERNSGYALNFGKGTSLLVTPHIQNPDPAVYQLRDS

SEQ ID NO: 32 - protein, 147 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGPDSNQPQHFGDGTRLSILEDLNKVFPPEVAVFE

SEQ ID NO: 33 - protein, 147 aa, Homo sapiens
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGQLRYGGATNKLIFGTGTLLAVQPNIQNPDPAVYQLRDS

SEQ ID NO: 34 - protein, 147 aa, Homo sapiens
MGCRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVGGASYEQYFGPGTRLTVTEDLKNVFPPEVAVFE

SEQ ID NO: 35 - protein, 144 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGPTYNQGGKLIFGQGTELSVKPNIQNPDPAVYQLRDS

SEQ ID NO: 36 - protein, 143 aa, Homo sapiens
MLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSARTAEVELFFGSGTQLSVLEDLNKVFPPEVAVFE

SEQ ID NO: 37 - protein, 150 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAVASWGSGSARQLTFGSGTQLTVLPDIQNPDPAVYQLRDS

SEQ ID NO: 38 - protein, 146 aa, Homo sapiens
MGTRLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLDAGYEQYFGPGTRLTVTEDLKNVFPPEVAVFE

SEQ ID NO: 39 - protein, 11 aa, Homo sapiens
MPFATPMEAEL

SEQ ID NO: 40 - protein, 9 aa, Homo sapiens
MPFATPMEA

SEQ ID NO: 41 - protein, 9 aa, Homo sapiens
FATPMEAEL

SEQ ID NO: 42 - protein, 10 aa, Homo sapiens
KEFTVSGNIL

SEQ ID NO: 43 - protein, 15 aa, Homo sapiens
TSYVKVLHHMVKISG

SEQ ID NO: 44 - protein, 9 aa, Homo sapiens
EVDPIGHLY

SEQ ID NO: 45 - protein, 10 aa, Homo sapiens
CAPNDYKLSF

SEQ ID NO: 46 - protein, 13 aa, Homo sapiens
CASSFSSDNEQFF

SEQ ID NO: 47 - protein, 12 aa, Homo sapiens
CATDSNDYKLSF

SEQ ID NO: 48 - protein, 11 aa, Homo sapiens
CASSWGGELFF

SEQ ID NO: 49 - protein, 17 aa, Homo sapiens
CAYRSDAGGTSYGKLTF

SEQ ID NO: 50 - protein, 16 aa, Homo sapiens
CASSPRTGGAGNTIYF

SEQ ID NO: 51 - protein, 17 aa, Homo sapiens
CAVRDIEVVNAGNMLTF

SEQ ID NO: 52 - protein, 13 aa, Homo sapiens
CASTDRVRYEQFF

SEQ ID NO: 53 - protein, 14 aa, Homo sapiens
CAVSDGGATNKLIF

SEQ ID NO: 54 - protein, 15 aa, Homo sapiens
CASSSGGRASFTQYF

SEQ ID NO: 55 - protein, 15 aa, Homo sapiens
CALSDPNNQGGKLIF

SEQ ID NO: 56 - protein, 14 aa, Homo sapiens
CASSQVRGTTEAFF

SEQ ID NO: 57 - protein, 14 aa, Homo sapiens
CAFLGSGNTGKLIF

SEQ ID NO: 58 - protein, 15 aa, Homo sapiens
CSAIRPSGGAFEQYF

SEQ ID NO: 59 - protein, 14 aa, Homo sapiens
CAARATSGTYKYIF

SEQ ID NO: 60 - protein, 14 aa, Homo sapiens
CSASFSRDIETQYF

SEQ ID NO: 61 - protein, 15 aa, Homo sapiens
CLVGPTYNQGGKLIF

SEQ ID NO: 62 - protein, 12 aa, Homo sapiens
CSARTAEVELFF

SEQ ID NO: 63 - protein, 16 aa, Homo sapiens
CAVASWGSGSARQLTF

SEQ ID NO: 64 - protein, 13 aa, Homo sapiens
CASSLDAGYEQYF

SEQ ID NO: 65 - protein, 11 aa, Homo sapiens
CAEPNTDKLIF

SEQ ID NO: 66 - protein, 13 aa, Homo sapiens
CATNYDRGYEQYF

SEQ ID NO: 67 - protein, 12 aa, Homo sapiens
CAVWATGNQFYF

SEQ ID NO: 68 - protein, 13 aa, Homo sapiens
CATSFDRGYEQYF

SEQ ID NO: 69 - protein, 14 aa, Homo sapiens
CVVNERNSGYALNF

SEQ ID NO: 70 - protein, 14 aa, Homo sapiens
CATSGPDSNQPQHF

SEQ ID NO: 71 - protein, 14 aa, Homo sapiens
CAVRLGDAGNMLTF

SEQ ID NO: 72 - protein, 15 aa, Homo sapiens
CASSVGRGMNTEAFF

SEQ ID NO: 73 - protein, 11 aa, Homo sapiens
CADNAGNMLTF

SEQ ID NO: 74 - protein, 14 aa, Homo sapiens
CSARDPGRNYGYTF

SEQ ID NO: 75 - protein, 11 aa, Homo sapiens
CGDNAGNMLTF

SEQ ID NO: 76 - protein, 14 aa, Homo sapiens
CSARDEDRPYGYTF

SEQ ID NO: 77 - protein, 16 aa, Homo sapiens
CAGQLRYGGATNKLIF

SEQ ID NO: 78 - protein, 14 aa, Homo sapiens
CASSVGGASYEQYF

SEQ ID NO: 79 - DNA, 31 bp, Artificial Sequence
  Oligonucleotide
gccaccatgatcctaaacaaagctctgmtgc

SEQ ID NO: 80 - DNA, 33 bp, Artificial Sequence
  Oligonucleotide
tatgcgatcgctcacaakggcccytggtgtctg

SEQ ID NO: 81 - DNA, 23 bp, Artificial Sequence
  Oligonucleotide
catcacaggaactttctgggctg

SEQ ID NO: 82 - DNA, 24 bp, Artificial Sequence
  Oligonucleotide
gctggtaggacaccgaggtaaagc

SEQ ID NO: 83 - DNA, 24 bp, Artificial Sequence
  Oligonucleotide
gctggtaagactcggaggtgaagc

SEQ ID NO: 84 - DNA, 20 bp, Artificial Sequence
  Oligonucleotide
cacgtggtcggggwagaagc

SEQ ID NO: 85 - DNA, 53 bp, Artificial Sequence
  Oligonucleotide
  misc_feature (26)..(30): n is a, c, g, or t
cgatccagactagacgctcaggaagnnnnnaagcagtggtatcaacgcagagt

SEQ ID NO: 86 - DNA, 53 bp, Artificial Sequence
  Oligonucleotide
caatatgtgaccgccgagtcccaggttagagtctctcagctggtacacggcag

SEQ ID NO: 87 - DNA, 50 bp, Artificial Sequence
  Oligonucleotide
caatatgtgaccgccgagtcccaggggctcaaacacagcgacctcgggtg

SEQ ID NO: 88 - DNA, 30 bp, Artificial Sequence
  Oligonucleotide
  misc_feature (1)..(5): n is a, c, g, or t
nnnnncgatccagactagacgctcaggaag

SEQ ID NO: 89 - DNA, 30 bp, Artificial Sequence
  Oligonucleotide
  misc_feature (1)..(5): n is a, c, g, or t
nnnnncaatatgtgaccgccgagtcccagg

SEQ ID NO: 90 - protein, 189 aa, Homo sapiens [sequence omitted]

SEQ ID NO: 91 - protein, 146 aa, Homo sapiens
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADMLDYQKVTFGIGTKLQVIPNIQNPDPAVYQLRDS

SEQ ID NO: 92 - protein, 146 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRRGGSEQYFGPGTRLTVTEDLKNVFPPEVAVFE

SEQ ID NO: 93 - protein, 151 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGQDSSYKLIFGSGTRLLVRPDIQNPDPAVYQLRDS

SEQ ID NO: 94 - protein, 147 aa, Homo sapiens
MGTRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVLGPNTEAFFGQGTRLTVVEDLNKVFPPEVAVFE

SEQ ID NO: 95 - protein, 152 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNAGGTSYGKLTFGQGTILTVHPNIQNPDPAVYQLRDS

SEQ ID NO: 96 - protein, 148 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSRRLPPPYGYTFGSGTRLTVVEDLNKVFPPEVAVFE

SEQ ID NO: 97 - protein, 150 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESSNTGKLIFGQGTTLQVKPDIQNPDPAVYQLRDS

SEQ ID NO: 98 - protein, 148 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSRREPPPYGYTFGSGTRLTVVEDLNKVFPPEVAVFE

SEQ ID NO: 99 - protein, 151 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREIRDSSYKLIFGSGTRLLVRPDIQNPDPAVYQLRDS

SEQ ID NO: 100 - protein, 147 aa, Homo sapiens
MGTRLLCCVAFCLLGAGPVDSGVTQTPKHLITATGQRVTLRCSPRSGDLSVYWYQQSLDQGLQFLIQHYNGEERAKGNILERFSAQQFPDLHSELNLSSLELGDSALYFCASSPAGPNTEAFFGQGTRLTVVEDLNKVFPPEVAVFE

SEQ ID NO: 101 - protein, 9 aa, Homo sapiens
SLLMWITQC

SEQ ID NO: 102 - protein, 10 aa, Homo sapiens
ILDTAGHEEY

SEQ ID NO: 103 - protein, 13 aa, Homo sapiens
CGADMLDYQKVTF

SEQ ID NO: 104 - protein, 12 aa, Homo sapiens
CASRRGGSEQYF

SEQ ID NO: 105 - protein, 15 aa, Homo sapiens
CAMREGQDSSYKLIF

SEQ ID NO: 106 - protein, 14 aa, Homo sapiens
CASSVLGPNTEAFF

SEQ ID NO: 107 - protein, 16 aa, Homo sapiens
CALSNAGGTSYGKLTF

SEQ ID NO: 108 - protein, 15 aa, Homo sapiens
CASSRRLPPPYGYTF

SEQ ID NO: 109 - protein, 14 aa, Homo sapiens
CALSESSNTGKLIF

SEQ ID NO: 110 - protein, 15 aa, Homo sapiens
CASSRREPPPYGYTF

SEQ ID NO: 111 - protein, 15 aa, Homo sapiens
CAMREIRDSSYKLIF

SEQ ID NO: 112 - protein, 14 aa, Homo sapiens
CASSPAGPNTEAFF